SEQUENCE LISTING (1 sequence)

SEQ ID NO: 1 - protein, 4 aa, Artificial Sequence
  Example of enzymatically degradable linkage.
LGPA

(19) United States
(12) Patent Application Publication
(10) Pub. No.: US 2009/0117070 A1
(43) Pub. Date: May 7, 2009

(54) Methods and Crosslinked Polymer Compositions for Cartilage Repair

(75) Inventors: George Y. Daniloff, Mountain View, CA (US); Philip M. Toleikis, Vancouver (CA); William L. Hunter, Vancouver (CA); Rui Avelar, Vancouver (CA)

Correspondence Address: SEED INTELLECTUAL PROPERTY LAW GROUP PLLC, 701 FIFTH AVENUE, SUITE 5400, SEATTLE, WA, 98104-7092, US

(73) Assignee: Angiotech Pharmaceuticals (US), Inc., North Bend, WA (US)

(21) Appl. No.: 11/571,048

(22) PCT Filed: Jun. 23, 2005
(86) PCT No.: PCT/US2005/022343

§ 371 Date: Dec. 20, 2006

Related U.S. Application Data
(60) Provisional application No. 60/582,651, filed on Jun. 23, 2004.

(51) Int. Cl. A61K 31/715; A61K 38/19; A61K 31/765; A61K 31/337; A61K 35/28; A61P 19/04; A61K 39/395; A61K 38/43; A61K 38/39

(52) U.S. Cl. 424/78.31; 514/2; 514/12; 514/54; 424/94.1; 514/449; 424/130.1; 424/549

(57) ABSTRACT

A method of repairing damaged cartilage and soft tissue in a patient is provided using a biocompatible, non-immunogenic composition. The composition comprises a hydrophilic polymer and a plurality of crosslinkable components having reactive functional groups. The composition used in the method may be loaded with biologically active agents for delivery to the damaged tissues. Kits for use in carrying out the method of the invention are also provided.

### CROSS-REFERENCE TO RELATED APPLICATIONS

This application, filed on Dec. 20, 2006, claims priority under 35 U.S.C. § 119(a) to PCT/US2005/022343, filed on Jun. 23, 2005, which claims priority under 35 U.S.C. § 119(e) to 60/582,651, filed on Jun. 23, 2004, both of which are incorporated by reference in their entireties herein.

### TECHNICAL FIELD

This invention relates generally to methods for repairing connective tissue (e.g., cartilage) using compositions comprised of a hydrophilic polymer and crosslinked biomaterials.

### BACKGROUND OF THE INVENTION

Successful human athletic performance requires the optimal function of our articulations. Proper joint function requires not only adequate strength and stability, but a smooth, gliding articular surface to allow an effortless range of motion. The tissue most responsible for creating the articular surface is hyaline cartilage; a tissue composed of chondrocytes (cartilage cells), extracellular matrix (type II collagen, aggrecan, glycosaminoglycans) and water. Dysfunction of this natural bearing surface results in pain, swelling, and limitation of function. Furthermore, injury to this structure may result in progressive degeneration into osteoarthritis. Full thickness cartilage defects are filled by granulation tissue, which is eventually replaced by fibrocartilage which does not have the same mechanical, structural, or functional properties as hyaline cartilage. For some cartilage injuries, surgical correction and repair is not even possible. For example, in the case of meniscal cartilage (the cartilage at the periphery of the joint surface), tears in the vascular zone may be repaired with sutures or with devices such as “meniscal arrows,” but tears in the non vascular zone or the “white on white” zone have little or no chance of repair and the damaged cartilage is often excised, thus potentially accelerating the development of osteoarthritis in the joint.

Damage to cartilage of the knee or other joints can result for many reasons. For example, cartilage can be damaged due to injury, degradation (e.g., due to osteoarthritis), disease (e.g. infection, rheumatoid arthritis and other rheumatic diseases), or due to a physical deformity that places an abnormal mechanical load on the joint. A particularly common type of cartilage injury is tearing of the meniscus in the knee. Unlike most tissues, cartilage has a limited capacity for regeneration once it is damaged. The limited ability for cartilage tissue to regenerate arises because adult chondrocytes, the cartilage-specific cells which give rise to normal cartilage tissue growth, are unable to reproduce and generate new cartilage in vivo.

Numerous alternative treatment strategies have been proposed for repairing or regenerating damaged articular cartilage. Some approaches have been based upon grafting of autologous (i.e., tissues derived from the patient themselves) chondral and osteochondral tissues or replacement tissue (i.e., neocartilge), which has been generated in vitro, to the native cartilage. Methods of in vivo articular cartilage repair include transplanting chondrocytes as injectable cells or as a composition of cells embedded in a three-dimensional scaffold (see e.g., Int'l Pat. Pub. No. WO 90/12603). Recent methods of articular cartilage repair have focused on biological resurfacing of cartilage defects with either a prosthetic device or with live chondrocytes. Resorbable collagen-containing membranes have been used in guided tissue regeneration (see e.g., U.S. Pat. No. 5,837,278 to Geistlich et al.).

Because of the inferior mechanical properties of most cartilage repair tissue that forms following osteochondral injury or surgical treatment of cartilage defects, investigators and surgeons have explored the use of a variety of cartilage grafts, including osteochondral autografts and allografts and periosteal and perichondrial grafts, to replace regions of damaged or lost articular (or meniscal) surface. More recently, several groups of investigators have developed methods of isolating chondrocytes or undifferentiated mesenchymal cells (or stem cells), growing them in culture, and then implanting them in the joint in a gel or other artificial matrix to replace damaged articular cartilage. Although significant progress has been made in the harvesting and culturing of cartilage tissue, successfully grafting the tissue back into the joint has remained a significant challenge. Problems include a lack of donor sites, an inability to attach and secure the cartilage to the underlying bone, grafting of the cartilage to the donor site and conversion of the hyaline cartilage to fibrocartilage or scar tissue after implantation.

Attempts have also been made to use synthetic matrix grafts (with or without cells and growth factors) that stimulate cartilage formation as another method of replacing regions of damaged or lost articular cartilage. The creation of synthetic matrices that vary in size and shape make it possible to fill any chondral defect precisely. A synthetic matrix provides a framework for cell migration and attachment and may give the implanted cartilage cells some protection from excessive loading. The cartilage, mesenchymal or stem cells included in these synthetic grafts may be autografts (from the same person), allografts (from a different person) or xenografts (from a different species; e.g., bovine or porcine). Many previously described synthetic matrices used to replace articular cartilage and to implant growth factors and/or cells contain reconstituted collagen as one of their structural components. Creating the ideal matrix composition and organization is critical for the success of cartilage transplantation, as it can influence cell migration, proliferation, and differentiation of the cartilage cells. There remains a significant unmet medical need to create an effective cartilage repair matrix to increase the success rate of cartilage repair procedures. The following invention details the composition and methods of use of such an implant material.

### SUMMARY OF THE INVENTION

The invention is directed to compositions and methods of repairing cartilage tissue using biocompatible compositions and for attaching tissues (such as cartilage, muscle, tendon and ligaments) to the underlying bone or periosteal tissue.

In one aspect of the invention, the compositions can be used for repairing injured cartilage tissue (e.g., articular or meniscal cartilage) at a treatment site. The treatment site may be, for example, in a joint (e.g., in the knee, shoulder, ankle, elbow, wrist, and the like) and the compositions may be used for the repair of articular cartilage defects and/or meniscal tears. The described compositions may facilitate growth of new cartilage tissue at the site of implantation. The described compositions can be used to facilitate attachment of connective tissue, such as cartilage, to the underlying bone. The described compositions can also facilitate the attachment of other connective tissues such as tendon, ligaments, fat, muscle or other tissue to underlying bone or periosteum during procedures such as facelifts, tendon and ligament repairs, soft tissue reconstructive procedures, and cosmetic implant procedures (such as breast implants and facial implants).

In another aspect of the invention, the present invention provides for methods of repairing injured cartilage in a joint of an animal, wherein the animal is administered an effective amount of a composition. The composition may, optionally, include a biologically active agent which aids in healing or regrowth of normal tissue (e.g., a cytokine or bone morphogenic factor).

The composition has a hydrophilic polymer and crosslinkable components that may be readily crosslinked upon admixture with an aqueous medium to provide a crosslinked composition suitable for use as a biomaterial. The composition is biocompatible, and does not leave any toxic, inflammatory, or immunogenic reaction products at the site of administration. Furthermore, as the composition is not subject to enzymatic cleavage by matrix metalloproteinases such as collagenase, it is not readily degradable in vivo. As a result, the composition may degrade more slowly than either the hydrophilic polymer component or the crosslinkable component as the two components will serve to mutually protect each other from the effects of metalloproteases or hydrolysis.

Accordingly, in one aspect of the invention, a method for tissue repair is provided utilizing a readily crosslinkable, biocompatible, composition to repair a cartilage defect or meniscal tear in a joint, such as the knee. Further in a similar aspect of the invention, a method for connective tissue repair is provided utilizing a readily crosslinkable, biocompatible, composition to attach muscle, tendon, ligament fat or an implanted prosthesis (e.g., breast implants or facial implants) to the underlying bone, periosteum or connective tissue. The composition is comprised of a hydrophilic polymer, a crosslinkable component A having m nucleophilic groups, wherein m≧2; and a crosslinkable component B having n electrophilic groups capable of reaction with the m nucleophilic groups to form covalent bonds, wherein n≧2 and m+n>4. In the composition, each of components A and B is biocompatible and nonimmunogenic, at least one of components A and B is a hydrophilic polymer, and admixture of components A and B in an aqueous medium results in crosslinking of the composition to give a biocompatible, nonimmunogenic, crosslinked matrix.

Each of the crosslinkable components may be polymeric, in which case at least two crosslinkable components are generally although not necessarily composed of a purely synthetic polymer rather than a naturally occurring or semi-synthetic polymer, wherein “semi-synthetic” refers to a chemically modified naturally occurring polymer. Alternatively, one or two of crosslinkable components A and B may be a low molecular weight crosslinking agent, typically an agent comprised of a hydrocarbyl moiety containing 2 to 14 carbon atoms and at least two functional groups, i.e., nucleophilic or electrophilic groups, depending on the component. For convenience, the term “polynucleophilic” will be used herein to refer to a compound having two or more nucleophilic moieties, and the term “polyelectrophilic” will be used to refer to a compound having two or more electrophilic moieties. The composition may also additionally comprise an optional third biocompatible and nonimmunogenic crosslinkable component C having at least one functional group selected from (i) nucleophilic groups capable of reacting with the electrophilic groups of component B and (ii) electrophilic groups capable of reacting with the nucleophilic groups of component A.

In yet another aspect of the present invention, there is provided a method for repairing damaged cartilage tissue in a patient, comprising the steps of: placing into contact with the damaged cartilage tissue a composition comprising the following components or a partial reaction product of the following components: (i) a first hydrophilic polymer; (ii) a crosslinkable component A having m nucleophilic groups, wherein m≧2; and (iii) a crosslinkable component B having n electrophilic groups capable of reaction with the m nucleophilic groups to form covalent bonds, wherein n≧2 and m+n>4, and further wherein each of components A and B is biocompatible and nonimmunogenic, and at least one of the components A and B is a second hydrophilic polymer, and reaction of the components results in a biocompatible, nonimmunogenic, crosslinked matrix. Within this aspect of the invention, the method has application where the cartilage is articular cartilage or more specifically where the cartilage is a meniscus or labrum. Within this aspect, the composition may be placed into contact with the damaged tissue via arthroscopic techniques.

In yet another aspect of the present invention, there is provided a method for repairing damaged soft tissues in a patient, comprising the steps of: placing into contact with the connective tissue (such as tendon, ligament, fat or soft tissue implant—such as a breast or facial implant) a composition comprising the following components or a partial reaction product of the following components: (i) a first hydrophilic polymer; (ii) a crosslinkable component A having m nucleophilic groups, wherein m≧2; and (iii) a crosslinkable component B having n electrophilic groups capable of reaction with the m nucleophilic groups to form covalent bonds, wherein n≧2 and m+n>4, and further wherein each of components A and B is biocompatible and nonimmunogenic, and at least one of the components A and B is a second hydrophilic polymer, and reaction of the components results in a biocompatible, nonimmunogenic, crosslinked matrix. Within this aspect of the invention, the method has application where the connective tissue is articular ligament, tendon, muscle, fat or a soft tissue implant (such as a cosmetic breast or facial implant). Within this aspect, the composition may be placed into contact with the connective tissue or the underlying bone, periosteum or connective tissue via arthroscopic techniques. Examples of such procedures include facelifts, implantation of cosmetic facial implants (synthetic implants such as facial, cheek, and chin implants; tissue reconstructions using muscle and/or skin flaps; collagen injections; fat/adipose tissue injections; hyaluronic acid injections) and implantation of cosmetic breast implants.

In the method of repairing damaged tissue described above, the first hydrophilic polymer may be synthetic or naturally occurring. Naturally occurring hydrophilic polymers contemplated within the present invention are selected from the group consisting of proteins, peptides, polysaccharides, lipids and derivatives thereof. Examples of polysaccharides include without limitation, carboxylated polysaccharides, aminated polysaccharides, glycosaminoglycans, and activated polysaccharides. Proteins that may be used in the claimed method include collagens, such as for example, nonfibrillar collagen selected from the group consisting of: type IV collagen, type VI collagen, and type VII collagen or fibrillar collagen. Nonfibrillar collagen may be chemically modified collagen such as for example, methylated collagen. Combinations of nonfibrillar and fibrillar collagen are also contemplated within the invention as is a mixture of particulate crosslinked fibrillar collagen and noncrosslinked fibrillar collagen. An example of a particulate crosslinked fibrillar collagen is glutaraldehyde-crosslinked collagen. Denatured collagen is also contemplated under the claimed method. In a preferred embodiment the composition may be comprised of particulate crosslinked fibrillar collagen comprising between about 25% to about 95% and the noncrosslinked fibrillar collagen comprising between about 5% to about 75% by weight of the composition.

The second hydrophilic polymer that may be used in the method for repairing damaged tissue as described above may be selected from the group consisting of polyalkeleneoxides, polyurethanes, polyesters, polyethers, polythioethers, polyamides, and derivatives, copolymers, and combinations thereof.

The crosslinked components A and B of the method of repairing damaged tissue may both be a polyalkyleneoxide, such as, for example, poly(ethylene glycol) and the two components may be the same or different. The components may also be in admixture, which may be in liquid or solid form.

A third crosslinkable component C is also contemplated within the method of repairing damaged tissue of the present invention. Component C is preferably biocompatible and nonimmunogenic with at least one functional group selected from (a) nucleophilic groups capable of reacting with the electrophilic groups of component B and, (b) electrophilic groups capable of reacting with the nucleophilic groups of component A, wherein the total number of functional groups on component C is represented by p, such that m+n+p>5.

In another embodiment of the method of repairing damaged tissue, component A has the structural formula (I) and component B has the structural formula (II)

R¹(-[Q¹]_{q}-X)ₘ (I)

R²(-[Q²]ᵣ-Y)ₙ (II)

wherein R¹ and R² are independently selected from the group consisting of C₂ to C₁₄ hydrocarbyl, heteroatom-containing C₂ to C₁₄ hydrocarbyl, hydrophilic polymers, and hydrophobic polymers; X represents one of the m nucleophilic groups of component A; Y represents one of the n electrophilic groups of component B; Q¹ and Q² are linking groups; and q and r are independently zero or 1.

Where the composition includes the third crosslinkable component C, component C has the structural formula (III)

R³(-[Q³]ₛ-Fn)ₚ (III)

wherein R³ is selected from the group consisting of C₂ to C₁₄ hydrocarbyl, heteroatom-containing C₂ to C₁₄ hydrocarbyl, hydrophilic polymers, and hydrophobic polymers; Fn represents a functional group on component C; and s is zero or 1.

Within the two embodiments of the invention described immediately above, at least one of R¹ and R² may be a synthetic hydrophilic polymer. For example, under the claimed method it is contemplated that R¹ may be a first synthetic hydrophilic polymer; and R² may be selected from the group consisting of (i) a second synthetic hydrophilic polymer that may or may not be the same as R¹ and (ii) C₂ to C₁₄ hydrocarbyl groups containing zero to 2 heteroatoms selected from N, O and S. The synthetic hydrophilic polymer is of a linear, branched, dendrimeric, hyperbranched, or star polymer and may be selected from the group consisting of: polyalkylene oxides; polyglycerols; poly(oxyalkylene)-substituted diol or polyol; polyacrylic acid and analogues thereof; polymaleic acid; polyacrylamides; poly(olefinic alcohol)s; poly(N-vinyl lactams); polyoxazolines; polyvinylamines; and copolymers thereof. Within this grouping, the polyalkylene oxide may be selected from the group consisting of polyethylene glycol and poly(ethylene oxide)-poly(propylene oxide) copolymers. The poly(oxyalkylene)-substituted diol or polyol may be selected from the group consisting of mono-poly(oxyalkylene)-substituted propylene glycol, di-(polyoxyalkylene)-substituted propylene glycol, mono-poly(oxyalkylene)-substituted trimethylene glycol, di-(polyoxyalkylene)-substituted trimethylene glycol, mono-poly(oxyalkylene)-substituted glycerol, di-(polyoxyalkylene)-substituted glycerol, and tri-(polyoxyalkylene)-substituted glycerol. The poly(acrylic acid) may be selected from the group consisting of poly(methacrylic acid), poly(hydroxyethylmethacrylate), poly(hydroxyethylacrylate), poly(methylalkylsulfoxide acrylates), poly(methylalkylsulfoxide methacrylates), and copolymers thereof. The polyacrylamide may be selected from the group consisting of poly(methacrylamide), poly(dimethylacrylamide), poly(N-isopropylacrylamide), and copolymers thereof. The poly(olefinic alcohol) may include polyvinyl alcohol or a copolymer thereof. The poly(N-vinyl lactam) may be selected from the group consisting of poly(vinyl pyrrolidone), poly(vinyl caprolactam), and copolymers thereof.

In another embodiment of the method of repairing damaged tissue, component A has the structural formula (I) and component B has the structural formula (II)

R¹(-[Q¹]_{q}-X)ₘ (I)

R²(-[Q²]ᵣ-Y)ₙ (II)

wherein R¹ and R² are independently selected from the group consisting of C₂ to C₁₄ hydrocarbyl, heteroatom-containing C₂ to C₁₄ hydrocarbyl, hydrophilic polymers, and hydrophobic polymers; X represents one of the m nucleophilic groups of component A; Y represents one of the n electrophilic groups of component B; Q¹ and Q² are linking groups; and q and r are independently zero or 1.

Where the composition includes the third crosslinkable component C, component C has the structural formula (III)

R³(-[Q³]ₛ-Fn)ₚ (III)

wherein R³ is selected from the group consisting of C₂ to C₁₄ hydrocarbyl, heteroatom-containing C₂ to C₁₄ hydrocarbyl, hydrophilic polymers, and hydrophobic polymers; Fn represents a functional group on component C; and s is zero or 1. Within this embodiment, it is preferred for r and s to be zero or for at least one of r and s to be 1.

Within the two embodiments of the invention described immediately above, the nucleophilic groups on component A may be selected from the group consisting of —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, and —C₅H₄N, wherein R⁴ and R⁵ are C₁-C₁₂ hydrocarbyl. In a preferred embodiment, the nucleophilic groups are selected from —NH₂ and —NHR⁴ and R⁴ is lower hydrocarbyl. The electrophilic groups on component B are preferably amino-reactive groups. In a preferred embodiment, the amino-reactive groups contain an electrophilically reactive carbonyl group susceptible to nucleophilic attack by a primary or secondary amine. Here, the amino-reactive groups may be carboxylic acid esters, carboxylic acids, or aldehydes.

In a preferred embodiment of the present invention, the composition comprises about 5-40% (w/w) of a combination of components A and B, wherein each of components A and B comprises poly(ethyleneoxide), and the first hydrophilic polymer is methylated collagen in aqueous solution at a concentration of about 15-25% (mg methylated collagen/ml aqueous solution). In a more preferred embodiment, the composition comprises about 10-20% (w/w) of a combination of components A and B, wherein each of components A and B comprises poly(ethyleneoxide), and the first hydrophilic polymer is methylated collagen in aqueous solution at a concentration of about 20% (methylated collagen/aqueous solution in mg/mL).

In another embodiment of the method of repairing damaged tissue, the first or second hydrophilic polymer may comprise a tissue reactive group, wherein the tissue reactive group is capable of reacting with a moiety at a tissue surface to provide for immobilization of the composition at the tissue surface.

In another aspect of the invention, composition may be biodegradable or bioerodible. In a preferred embodiment, the composition has an initial volume prior to contact with the cartilage or other connective tissue and a final volume after contact with the cartilage or other connective tissue, wherein the final volume is about 10% to about 200% of the initial volume.

In yet another aspect of the invention, the crosslinked matrix has an elastic modulus ranging from about 2 N/cm² to about 40 N/cm² and a tensile strength ranging from about 1.5 N/cm² to about 70 N/cm².

In a further aspect of the invention, the composition further comprises a biologically active agent, which may facilitate tissue healing and regeneration. The biologically active agent may be selected from the group consisting of enzymes, receptor antagonists, or agonists, hormones, growth factors, small molecules which stimulate cell migration, adhesion and/or proliferation, autogenous bone marrow, antibiotics, antimicrobial agents, and antibodies. The small molecules may be selected from the group consisting of dexamethasone, isotretinoin (13-cis retinoic acid), 17-β-estradiol, estradiol, 1-α-25 dihydroxyvitamin D₃, diethylstibesterol, cyclosporin A, L-NAME (a non-selective inhibitor of nitric oxide synthase that has been used experimentally to induce hypertension), all-trans retinoic acid (ATRA), and analogues and derivatives thereof. The biologically active agent may also be a cytokine, which may be selected from the group consisting of TNFα, NGF, GM-CSF, IL-1, IL-1-β, IL-8, IL-6, growth hormone, transforming growth factors (TGFs), fibroblast growth factors (FGFs), platelet derived growth factors (PDGFs), epidermal growth factors (EGFs), RGD (Arg-Gly-Asp) peptide, connective tissue activated peptides (CTAPs), osteogenic factors, members of the TGF supergene family, and biologically active analogues, fragments, and derivatives thereof. In a preferred embodiment, the cytokine is present in the composition at a concentration of about 0.0001 μg/mL to about 20 mg/mL. The biologically active agent may also be a bone morphogenic protein, which may be one of BMP-2, BMP-3, BMP-4, BMP-5, BMP-6, or BMP-7 or an analogue or derivative thereof. In a preferred embodiment, the bone morphogenic protein is present in the composition at a concentration of about 0.0001 μg/mL to about 25 mg/mL. The biologically active agent may also be a growth factor, which may be selected from the group consisting of heparin-binding growth factors, inhibins, growth differentiating factors, and activins.

In another aspect of the invention, the composition may further comprise an agent that stimulates processes involving tissue regeneration. The agent may be, for example, a viable tissue cell. The agent may serve to stimulate cellular proliferation, cell migration, cell adhesion, or a combination thereof. Agents contemplated under the invention may be selected from the group consisting of dexamethasone, isotretinoin (13-cis retinoic acid), 17-β-estradiol, estradiol, 1-α-25 dihydroxyvitamin D₃, diethylstibesterol, cyclosporin A, L-NAME (a non-selective inhibitor of nitric oxide synthase), all-trans retinoic acid (ATRA), and analogues and derivatives thereof. In a preferred embodiment, the agent is present in the composition at a concentration of about 0.1 μg/mL to about 25 mg/mL.

In yet another aspect of the invention, there is provided a method of repairing damaged cartilage or other connective tissue in a patient comprising (a) providing a flowable mixture comprising the following components or a partial reaction product of the following components: (i) a first hydrophilic polymer; (ii) a crosslinkable component A having m nucleophilic groups, wherein m≧2; and (iii) a crosslinkable component B having n electrophilic groups capable of reaction with the m nucleophilic groups to form covalent bonds, wherein n≧2 and m+n>4; wherein each of components A and B is biocompatible and nonimmunogenic, and at least one of components A and B is a second hydrophilic polymer; (b) applying the flowable mixture to damaged cartilage or other connective tissue; and (c) irrigating the applied mixture with an aqueous initiating buffer, to form a biocompatible, nonimmunogenic, crosslinked matrix. Within this aspect of the invention, the aqueous initiating buffer may be a basic buffer and the reaction mixture is applied to the damaged cartilage tissue as a viscous liquid, partially polymerized gel, suspension, or as a spray. Embodiments of this aspect of the invention will be the same as those described above for the related method. As in the previously described method, this method may also comprise a third crosslinkable component C that is biocompatible and nonimmunogenic and has at least one functional group selected from (a) nucleophilic groups capable of reacting with the electrophilic groups of component B and, (b) electrophilic groups capable of reacting with the nucleophilic groups of component A, wherein the total number of functional groups on component C is represented by p, such that m+n+p>5.

Similarly, this method may include an embodiment wherein component A has the structural formula (I) and component B has the structural formula (II)

R¹(-[Q¹]_{q}-X)ₘ (I),

R²([Q²]ᵣ-Y)ₙ (II)

wherein R₁ and R₂ are independently selected from the group consisting of C₂ to C₁₄ hydrocarbyl, heteroatom-containing C₂ to C₁₄ hydrocarbyl, hydrophilic polymers, and hydrophobic polymers; X represents one of the m nucleophilic groups of component A; Y represents one of the n electrophilic groups of component B; Q¹ and Q² are linking groups; and q and r are independently zero or 1.

Where the method includes the third crosslinkable component C, component C has the structural formula (III)

R³(-[Q³]ₛ-Fn)ₚ (III)

wherein R³ is selected from the group consisting of C₂ to C₁₄ hydrocarbyl, heteroatom-containing C₂ to C₁₄ hydrocarbyl, hydrophilic polymers, and hydrophobic polymers; Fn represents a functional group on component C; and s is zero or 1.

Within the two embodiments described immediately above, R¹ and R² are as previously described. In a preferred embodiment, component A has the structural formula (I) and component B has the structural formula (II)

R¹(-[Q¹]_{q}-X)ₘ (I)

R²(-[Q²]ᵣ-Y)ₙ (II)

wherein R¹ and R² are independently selected from the group consisting of C₂ to C₁₄ hydrocarbyl, heteroatom-containing C₂ to C₁₄ hydrocarbyl, hydrophilic polymers, and hydrophobic polymers; X represents one of the m nucleophilic groups of component A; Y represents one of the n electrophilic groups of component B; Q¹ and Q² are linking groups; and q and r are independently zero or 1. Where the method includes the third crosslinkable component, component C has the structural formula (III)

R³(-[Q³]ₛ-Fn)ₚ (III)

wherein R³ is selected from the group consisting of C₂ to C₁₄ hydrocarbyl, heteroatom-containing C₂ to C₁₄ hydrocarbyl, hydrophilic polymers, and hydrophobic polymers; Fn represents a functional group on component C; and s is zero or 1. Within these preferred embodiments, it is preferred for r and s to be zero and more preferred for at least one of r and s to be 1. Further preferred embodiments of this aspect of the invention are comparable to those previously described.

In yet another aspect of the invention, there is provided a method of repairing damaged cartilage or other connective tissue in a patient comprising the steps of: placing into contact with the damaged tissue a composition comprising the following components or a partial reaction product of the following components: (i) methylated collagen; (ii) (pentaerythritol tetrakis[mercaptoethyl poly(oxyethylene) ether]; and (iii) pentaerythritol tetrakis [1-(1′-oxo-5-succimidylpentanoate)-2-poly(oxyethylene) ether], wherein reaction of the components results in a biocompatible, nonimmunogenic, crosslinked matrix.

In still another aspect of the invention, there is provided a method of repairing damaged cartilage or other connective tissue in a patient comprising the steps of: placing into contact with the damaged tissue a composition comprising the following components or a partial reaction product of the following components: (i) methylated collagen; (ii) (pentaerythritol tetrakis[mercaptoethyl poly(oxyethylene) ether]; and (iii) pentaerythritol tetrakis [1-(1′-oxo-5-succimidylpentanoate)-2-poly(oxyethylene) ether], wherein reaction of the components results in a biocompatible, nonimmunogenic, crosslinked matrix.

In the aforementioned methods for repairing damaged cartilage tissue, damaged connective tissue, and/or damaged soft tissue in a patient, the compositions may comprise a biologically active agent, which may be delivered to the damaged cartilage tissue, damaged connective tissue, and/or damaged soft tissue in the patient. For delivery to the tissues, the composition may be in the form of microparticles, nanoparticles, microemulsions, emulsions, liposomes and micelles. A preferred biologically active agent for use in the methods of the present invention is an angiogenesis inhibitor. Another preferred biologically active agent for use in the methods of the present invention is paclitaxel or an analogue or derivative thereof. Additionally, other biologically active agents incorporated in the compositions of the present invention may facilitate tissue healing and regeneration.

In a further aspect of the invention, there is provided a kit for repairing damaged cartilage or other connective tissue, wherein the kit comprises: (a) a first hydrophilic polymer; (b) a crosslinkable component A having m nucleophilic groups, wherein m≧2; and (c) a crosslinkable component B having n electrophilic groups capable of reaction with the m nucleophilic groups to form covalent bonds, wherein n≧2 and m+n>4, wherein each of components A and B is biocompatible and nonimmunogenic, and at least one of components A and B is a second hydrophilic polymer, and reaction of the components results in a biocompatible, nonimmunogenic, crosslinked matrix.

In a preferred embodiment of the kit, each of components A and B comprise polyalkeleneoxide. In a more preferred embodiment, the polyalkyleneoxide is a poly(ethylene oxide). Within the kit, it is contemplated that components A and B may be the same or different. Components A and B may also be in admixture in a liquid or a solid form.

In yet another aspect of the invention, the kit further comprises a device for mixing (a), (b), and (c) and delivering (a), (b), and (c) or a partial reaction product thereof to the damaged cartilage or other connective tissue. The device may be configured to spray material onto a surface of the damaged cartilage tissue, muscle, periosteum, ligament, tendon fat, and/or soft tissue implant. For example, the device may be configured to deliver material onto a surface of the damaged cartilage or other connective tissue as a liquid, gel, or suspension.

In a preferred embodiment of the kit, the first hydrophilic polymer is methylated collagen dissolved or suspended in aqueous solution of pH less than 7.

In still a further aspect of the invention, the kit further comprises an additional component (d), which comprises an aqueous solution of pH greater than 7.

In yet another aspect of the invention, there is provided a kit for repairing damaged cartilage or other connective tissue, wherein the kit comprises: (a) an aqueous solution of methylated collagen, the solution having a pH of less than 7; (b) (pentaerythritol tetrakis[mercaptoethyl poly(oxyethylene) ether]; and (c) pentaerythritol tetrakis [1-(1′-oxo-5-succimidylpentanoate)-2-poly(oxyethylene) ether], wherein (b) and (c) are in admixture in solid form; and (d) an aqueous solution having a pH of greater than 7.

The ultimate goal in the surgical treatment of articular cartilage lesions is the reproduction of viable hyaline cartilage bound to a restored subchondral bone plate and to the surrounding hyaline cartilage and/or to restore a hyaline cartilage interface between torn meniscal or labral edges. Traditionally, debridement has been performed mechanically with the use of rotary power shavers or other hand instruments. Any conventional surgical procedure may be used to access the injured cartilage tissue. For example, the composition may be applied into the cartilage defect (lesion) to cover all surfaces of the cartilage defects and fill the defect volume such that the composition provides a continuum between surfaces of the cartilage defect. Depending on the severity of the defect, the composition may be applied onto the subchondral bone and/or calcified cartilage. The method is applicable to a wide variety of cartilage types, including but not limited to, cartilage in the knee, elbow, ankle, shoulder, wrist, finger joint, and the like. In the case of the meniscus, cartilage tears in areas of vascular supply may undergo suturing or reapproximation of the torn edges with a devices such as a meniscal arrow, but tears in the avascular zone generally have no hope of repair and are cut out.

The ultimate goal in the surgical treatment of connective tissue lesions is the formation of strong, lasting bonds between the adjacent tissues. For example, tendons and ligaments need to graft to, and integrate with, muscle, bone tissue or other connective tissues; cosmetic implants need to attach to and integrate with the chest wall or the facial bones; muscle and soft tissue flaps must be attached to underlying bone or muscle during facelifts and other reconstructive procedures in plastic surgery. Any conventional surgical procedure may be used to access the injured connective tissue. For example, the composition may be applied into the soft tissue defect (lesion) to cover all or only portions of surfaces of the defect or implant.

### BRIEF DESCRIPTION OF THE DRAWINGS

FIGS. 1 to 10 schematically illustrate reaction of various polyelectrophilic components with substituted polyethylene glycol (PEG) as a representative polynucleophile. In FIGS. 1-10, the polyelectrophilic components are composed of a pentaerythritol core with each of the four hydroxyl groups substituted with PEG, and with each PEG branch terminated with a reactive electrophilic group.

FIG. 11 is a graph showing the effect of cyclosporin A on proliferation of human smooth muscle cells.

FIG. 12 is a graph showing the effect of dexamethasone on proliferation of human fibroblasts.

FIG. 13 is a graph showing the effect of all-trans retinoic acid (ATRA) on proliferation of human smooth muscle cells.

FIG. 14 is a graph showing the effect of isotretinoin on proliferation of human smooth muscle cells.

FIG. 15 is a graph showing the effect of 17-β-estradiol on proliferation of human fibroblasts.

FIG. 16 is a graph showing the effect of 1α, 25-dihydroxy-vitamin D₃ on proliferation of human smooth muscle cells.

FIG. 17 is a graph showing the effect of PDGF-BB on smooth muscle cell migration.

### DETAILED DESCRIPTION OF THE INVENTION

### Definitions and Nomenclature

Before describing the present invention in detail, it is to be understood that unless otherwise indicated this invention is not limited to particular compositional forms, crosslinkable components, crosslinking techniques, or methods of use, as such may vary. It is also to be understood that the terminology used herein is for the purpose of describing particular embodiments only, and is not intended to be limiting.

As used in this specification and the appended claims, the singular forms “a,” “an,” and “the” include plural referents unless the context clearly dictates otherwise. Thus, for example, “a crosslinkable component” refers not only to a single crosslinkable component but also to a combination of two or more different crosslinkable components; “a hydrophilic polymer” refers to a combination of hydrophilic polymers as well as to a single hydrophilic polymer, and the like. Any concentration ranges recited herein are to be understood to include concentrations of any integer within that range and fractions thereof, such as one tenth and one hundredth of an integer, unless otherwise indicated. Also, any number range recited herein relating to any physical feature, such as polymer subunits, size or thickness, are to be understood to include any integer within the recited range, unless otherwise indicated. As used herein, the term “about” means±15%. As used herein, the terms “average” or “mean” include the arithmetic mean as well as any appropriate weighted averages such as are used in the expression of polymeric molecular weight or particle size distributions.

Unless defined otherwise, all technical and scientific terms used herein have the meaning commonly understood by one of ordinary skill in the art to which the invention pertains. Although any methods and materials similar or equivalent to those described herein may be useful in the practice or testing of the present invention, preferred methods and materials are described below. All patents, patent applications and other publications mentioned herein are incorporated herein by reference. Specific terminology of particular importance to the description of the present invention is defined below.

As used herein, the terms “bioadhesive,” “biological adhesive,” and “surgical adhesive” are used interchangeably to refer to biocompatible compositions capable of effecting temporary or permanent attachment between the surfaces of two native tissues, or between a native tissue surface and either a non-native tissue surface or a surface of a synthetic implant.

The term “surgically acceptable” refers to those items, e.g., implants, that are biocompatible, and are otherwise acceptable for surgical use.

“Soft Tissue Implant” refers to a medical device or implant that includes a volume replacement material for augmentation or reconstruction to replace a whole or part of a living structure. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants include breast implants, chin implants, calf implants, cheek implants and other facial implants, buttocks implants, and nasal implants.

The term “crosslinked” herein refers to a composition containing intermolecular crosslinks and optionally intramolecular crosslinks as well, arising from the formation of covalent bonds. Covalent bonding between two crosslinkable components may be direct, in which case an atom in one component is directly bound to an atom in the other component, or it may be indirect, through a linking group. A crosslinked matrix may, in addition to covalent bonds, also include intermolecular and/or intramolecular noncovalent bonds such as hydrogen bonds and electrostatic (ionic) bonds. The term “crosslinkable” refers to a component or compound that is capable of undergoing reaction to form a crosslinked composition.

The terms “nucleophile” and “nucleophilic” refer to a functional group that is electron rich, has an unshared pair of electrons acting as a reactive site, and reacts with a positively charged or electron-deficient site, generally present on another molecule.

The terms “electrophile” and “electrophilic” refer to a functional group that is susceptible to nucleophilic attack, i.e., susceptible to reaction with an incoming nucleophilic group. Electrophilic groups herein are positively charged or electron-deficient, typically electron-deficient.

The term “activated” refers to a modification of an existing functional group to generate or introduce a new reactive functional group from the prior existing functional group, wherein the new reactive functional group is capable of undergoing reaction with another functional group to form a covalent bond. For example, a component containing carboxylic acid (—COOH) groups can be activated by reaction with N-hydroxysuccinimide (also referred to as NHS) or N-hydroxysulfosuccinimide (also referred to as NHSS) using known procedures, to form an activated carboxylate (which is a reactive electrophilic group), i.e., an N-hydroxysuccinimide ester or an N-hydroxysulfosuccinimide ester, respectively. In another example, carboxylic acid groups can be activated by reaction with an acyl halide, e.g., an acyl chloride, again using known procedures, to provide an activated electrophilic group in the form of an anhydride.

The terms “hydrophilic” and “hydrophobic” are generally defined in terms of a partition coefficient P, which is the ratio of the equilibrium concentration of a compound in an organic phase to that in an aqueous phase. A hydrophilic compound has a log P value less than 1.0, typically less than about −0.5, where P is the partition coefficient of the compound between octanol and water, while hydrophobic compounds will generally have a log P greater than about 3.0, typically greater than about 5.0. Preferred crosslinkable components herein are hydrophilic, although as long as the crosslinkable composition as a whole contains at least one hydrophilic component, crosslinkable hydrophobic components may also be present. The terms “hydrophilic” and “hydrophobic” also may be defined in terms of an HLB value, i.e., a hydrophilic lipophilic balance. A high HLB value indicates a hydrophilic compound, while a low HLB value characterizes a hydrophobic compound. HLB values are well known in the art, and generally range from 1 to 18. Preferred crosslinkable components herein are hydrophilic, although as long as the crosslinkable composition as a whole contains at least one hydrophilic component, crosslinkable hydrophobic components may also be present.

The term “polymer” is used not only in the conventional sense to refer to molecules composed of repeating monomer units, including homopolymers, block copolymers, random copolymers, and graft copolymers, but also, as indicated in commonly owned U.S. Pat. No. 6,323,278 to Rhee et al. to refer to polyfunctional small molecules that do not contain repeating monomer units but are “polymeric” in the sense of being “polyfunctional,” i.e., containing two or more functional groups. Accordingly, it will be appreciated that when the term “polymer” is used, difunctional and polyfunctional small molecules are included. Such moieties include, by way of example: the difunctional electrophiles disuccinimidyl suberate (DSS), bis(sulfosuccinimidyl) suberate (BS³), dithiobis(succinimidylpropionate) (DSP), bis(2-succinimidooxy-carbonyloxy)ethyl sulfone (BSOCOES), 3,3′-dithiobis(sulfosuccinimidylpropionate (DTSSP); and the di polyfunctional nucleophiles ethylenediamine (H₂N—CH₂—CH₂—NH₂), tetramethylene diamine (H₂N—[CH₂]₄—NH₂), pentamethylene diamine (cadaverine) (H₂N—[CH₂]₅—NH₂), hexamethylene diamine (H₂N—[C₂]₆—NH₂), bos(2-aminoethyl)amine (HN—[CH₂—CH₂—NH₂]₂), and tris (2-aminoethyl)amine (N—[C₂—CH₂—NH₂]₃). All suitable polymers herein are nontoxic, non-inflammatory and nonimmunogenic, and will preferably be essentially nondegradable in vivo over a period of up to 30 days in vivo.

The term “synthetic” to refer to various polymers herein is intended to mean “chemically synthesized.” Therefore, a synthetic polymer in the present compositions may have a molecular structure that is identical to a naturally occurring polymer, but the polymer, as incorporated in the compositions of the invention, has been chemically synthesized in the laboratory or industrially. “Synthetic” polymers also include semi-synthetic polymers, i.e., naturally occurring polymers, obtained from a natural source, that have been chemically modified in some way. Generally, however, the synthetic polymers herein are purely synthetic, i.e., they are neither semi-synthetic nor have a structure that is identical to that of a naturally occurring polymer.

The term “synthetic hydrophilic polymer” as used herein refers to a synthetic polymer composed of molecular segments that render the polymer as a whole “hydrophilic,” as defined above. Preferred polymers are highly pure or are purified to a highly pure state such that the polymer is or is treated to become pharmaceutically pure. Most hydrophilic polymers can be rendered water soluble by incorporating a sufficient number of oxygen (or less frequently nitrogen) atoms available for forming hydrogen bonds in aqueous solutions. Hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di tri-polyoxyethylated glycerol, mono di-polyoxy-ethylated propylene glycol, and mono di-polyoxyethylated trimethylene glycol; polyoxyethylated sorbitol, polyoxyethylated glucose; acrylic acid polymers and analogues and copolymers thereof, such as polyacrylic acid, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines.

Hydrophobic polymers, including low molecular weight polyfunctional species, can also be used in the crosslinkable compositions of the invention. Hydrophobic polymers preferably contain, or can be derivatized to contain, two or more electrophilic groups, such as succinimidyl groups, most preferably, two, three, or four electrophilic groups. Generally, “hydrophobic polymers” herein contain a relatively small proportion of oxygen and/or nitrogen atoms. Preferred hydrophobic polymers for use in the invention generally have a carbon chain that is no longer than about 14 carbons. Polymers having carbon chains substantially longer than 14 carbons generally have very poor solubility in aqueous solutions and, as such, have very long reaction times when mixed with aqueous solutions of synthetic polymers containing multiple nucleophilic groups.

The term “collagen” as used herein refers to all forms of collagen, including those, which have been processed or otherwise modified. Preferred collagens do not posses telopeptide regions (“atelopeptide collagen”), are soluble, and may be in fibrillar or non-fibrillar form. Type I collagen is best suited to most applications involving bone or cartilage repair; however, other forms of collagen are also useful in the practice of the invention, and are not excluded from consideration here. Collagen crosslinked using heat, radiation, or chemical agents such as glutaraldehyde may also be used to form particularly rigid crosslinked compositions. Collagen used in connection with the preferred embodiments of the invention is in a pharmaceutically pure form such that it can be incorporated into a human body for the intended purpose.

Those of ordinary skill in the art will appreciate that synthetic polymers such as polyethylene glycol cannot be prepared practically to have exact molecular weights, and that the term “molecular weight” as used herein refers to the weight average molecular weight of a number of molecules in any given sample, as commonly used in the art. Thus, a sample of PEG 2,000 might contain a statistical mixture of polymer molecules ranging in weight from, for example, 1,500 to 2,500 daltons with one molecule differing slightly from the next over a range. Specification of a range of molecular weights indicates that the average molecular weight may be any value between the limits specified, and may include molecules outside those limits. Thus, a molecular weight range of about 800 to about 20,000 indicates an average molecular weight of at least about 800, ranging up to about 20 kDa.

The term “cytokine” is used to describe biologically active molecules including growth factors and active peptides, which aid in healing or regrowth of normal tissue. The function of cytokines is two-fold: 1) they can incite local cells to produce new collagen or tissue, or 2) they can attract cells to the site in need of correction. As such, cytokines serve to encourage “biological anchoring” of the collagen implant within the host tissue. As previously described, the cytokines can either be admixed with the collagen-polymer conjugate or chemically coupled to the conjugate. For example, one may incorporate cytokines such as epidermal growth factor (EGF), transforming growth factor (TGF)-α, TGF-β (including any combination of TGF-βs), TGF-β1, TGF-β2, platelet derived growth factor (PDGF-AA, PDGF-AB, PDGF-BB), acidic fibroblast growth factor (FGF), basic FGF, connective tissue activating peptides (CTAP), β-thromboglobulin, insulin-like growth factors, tumor necrosis factors (TNF), interleukins, colony stimulating factors (CSFs), erythropoietin (EPO), RGD (Arg-Gly-Asp) sequence, nerve growth factor (NGF), interferons (IFN) bone morphogenic protein (BMP), osteogenic factors, and the like. Incorporation of cytokines and appropriate combinations of cytokines can facilitate the regrowth and remodeling of the implant into normal bone tissue, or may be used in the treatment of wounds.

The term “effective amount” refers to the amount of composition required in order to obtain the effect desired. Thus, a “tissue growth-promoting amount” of a composition refers to the amount needed in order to stimulate tissue growth to a detectable degree. Tissue, in this context, includes connective tissue, bone, cartilage, epidermis and dermis, blood, and other tissues. The actual amount that is determined to be an effective amount will vary depending on factors such as the size, condition, sex, and age of the patient and can be more readily determined by the caregiver.

The term “suitable fibrous material” as used herein, refers to a fibrous material which is substantially insoluble in water, non-immunogenic, biocompatible, and immiscible with the crosslinkable compositions of the invention. The fibrous material may comprise any of a variety of materials having these characteristics and may be combined with crosslinkable compositions herein in order to form and/or provide structural integrity to various implants or devices used in connection with medical and pharmaceutical uses. For example, the crosslinkable compositions of the invention can be coated on the “suitable fibrous material,” which can then be wrapped around a bone to provide structural integrity to the bone. Thus, the “suitable fibrous material” is useful in forming “solid implants”.

The term “in situ” as used herein means at the site of administration. Thus, the injectable reaction mixture compositions are injected or otherwise applied to a specific site within a patient's body, e.g., the locus of the tissue defect, and allowed to crosslink at the site of injection or application.

The term “aqueous medium” includes solutions, suspensions, dispersions, colloids, and the like containing water.

The terms “active agent,” “biologically active agent,” and “therapeutic agent” are used interchangeably herein to refer generally to a chemical material or compound suitable for administration to a patient and that induces a desired effect. The terms include agents that are therapeutically effective as well as prophylactically effective. Also included are derivatives and analogues of those compounds or classes of compounds specifically mentioned that also induce the desired effect. The terms refer more specifically to an organic molecule that exerts biological effects in vivo. Examples of biologically active agents include, without limitation, enzymes, receptor antagonists, or agonists, hormones, growth factors, small molecules which stimulate cell migration, adhesion and/or proliferation, autogenous bone marrow, antibiotics, antimicrobial agents and antibodies. The terms are also intended to encompass various cell types and genes that can be incorporated into the compositions of the invention. In one aspect, the active agents, biologically active agents, and/or therapeutic agents are selected to improve the tissue regenerative function of the composition or to reduce side effects associated with the implantation of a composition.

The term “hydrogel” is used in the conventional sense to refer to water-swellable polymeric matrices that can absorb a substantial amount of water to form elastic gels, wherein “matrices” are three-dimensional networks of macromolecules held together by covalent or noncovalent crosslinks. Upon placement in an aqueous environment, dry hydrogels swell to the extent allowed by the degree of cross-linking.

With regard to nomenclature pertinent to molecular structures, the following definitions apply:

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl and the like. Generally, although again not necessarily, alkyl groups herein contain 1 to about 12 carbon atoms. The term “lower alkyl” intends an alkyl group of one to six carbon atoms, preferably one to four carbon atoms. “Substituted alkyl” refers to alkyl substituted with one or more substituent groups. “Alkylene,” “lower alkylene,” and “substituted alkylene” refer to divalent alkyl, lower alkyl, and substituted alkyl groups, respectively.

The term “aryl” as used herein, and unless otherwise specified, refers to an aromatic substituent containing a single aromatic ring or multiple aromatic rings that are fused together, linked covalently, or linked to a common group such as a methylene or ethylene moiety. The common linking group may also be a carbonyl as in benzophenone, an oxygen atom as in diphenylether, or a nitrogen atom as in diphenylamine. Preferred aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl in which at least one carbon atom is replaced with a heteroatom. The terms “arylene” and “substituted arylene” refer to divalent aryl and substituted aryl groups as just defined.

The term “heteroatom-containing” as in a “heteroatom-containing hydrocarbyl group” refers to a molecule or molecular fragment in which one or more carbon atoms is replaced with an atom other than carbon, e.g., nitrogen, oxygen, sulfur, phosphorus or silicon.

“Hydrocarbyl” refers to univalent hydrocarbyl radicals containing 1 to about 30 carbon atoms, preferably 1 to about 24 carbon atoms, most preferably 1 to about 12 carbon atoms, including branched or unbranched, saturated or unsaturated species, such as alkyl groups, alkenyl groups, aryl groups, and the like. The term “lower hydrocarbyl” intends a hydrocarbyl group of one to six carbon atoms, preferably one to four carbon atoms. The term “hydrocarbylene” intends a divalent hydrocarbyl moiety containing 1 to about 30 carbon atoms, preferably 1 to about 24 carbon atoms, most preferably 1 to about 12 carbon atoms, including branched or unbranched, saturated or unsaturated species, or the like. The term “lower hydrocarbylene” intends a hydrocarbylene group of one to six carbon atoms, preferably one to four carbon atoms. “Substituted hydrocarbyl” refers to hydrocarbyl substituted with one or more substituent groups, and the terms “heteroatom-containing hydrocarbyl” and “heterohydrocarbyl” refer to hydrocarbyl in which at least one carbon atom is replaced with a heteroatom. Similarly, “substituted hydrocarbylene” refers to hydrocarbylene substituted with one or more substituent groups, and the terms “heteroatom-containing hydrocarbylene” and “heterohydrocarbylene” refer to hydrocarbylene in which at least one carbon atom is replaced with a heteroatom. If not otherwise indicated, “hydrocarbyl” indicates unsubstituted hydrocarbyl, substituted hydrocarbyl, heteroatom-containing hydrocarbyl, and substituted heteroatom-containing hydrocarbyl. Unless otherwise indicated, the terms “hydrocarbyl” and “hydrocarbylene” include substituted hydrocarbyl and substituted hydrocarbylene, heteroatom-containing hydrocarbyl and heteroatom-containing hydrocarbylene, and substituted heteroatom-containing hydrocarbyl and substituted heteroatom-containing hydrocarbylene, respectively.

By “substituted” as in “substituted hydrocarbyl,” “substituted alkyl,” and the like, as alluded to in some of the aforementioned definitions, is meant that in the hydrocarbyl, alkyl, or other moiety, at least one hydrogen atom bound to a carbon atom is replaced with one or more substituents that are functional groups such as alkoxy, hydroxy, halo, nitro, and the like. Unless otherwise indicated, it is to be understood that specified molecular segments can be substituted with one or more substituents that do not compromise a compound's utility. For example, “succinimidyl” is intended to include unsubstituted succinimidyl as well as sulfosuccinimidyl and other succinimidyl groups substituted on a ring carbon atom, e.g., with alkoxy substituents, polyether substituents, or the like.

### Repair of Damaged Connective Tissue

1. Cartilage, Tendons, and Ligaments

The present compositions may be used to repair a variety of cartilage types. In one aspect, the use of the composition is in the repair of damaged (e.g., torn) cartilage (i.e., cartilage that covers the articular surfaces of the bones participating in a synovial joint) or meniscal cartilage which can sit on an articular cartilage surface (e.g., meniscal cartilage in the knee, the labrum in the hip and shoulder). Although the methods are described for using the present compositions for the repair of cartilage, these compositions may be used to repair a variety of other connective tissue types, including ligaments, tendons, and bone. In any of the connective tissue injuries where of mechanical forces applied during body movement may effect the tissue healing and regeneration into its functional tissue phenotype (bone, cartilage, tendon), these compositions may be used to approximate tissue surfaces and provide a conduit for enhanced healing of the damaged connective tissue.

The present compositions may be also be used to repair a variety of connective tissue types. In one aspect, the use of the composition is in the repair of damaged (e.g., torn) tendons, ligaments or muscle tissue. In any of the connective tissue injuries where of mechanical forces applied during body movement may affect the tissue healing and regeneration into its functional tissue phenotype (bone, muscle, tendon, ligament), these compositions may be used to approximate tissue surfaces and provide a conduit for enhanced healing of the damaged connective tissue. The ultimate goal in the surgical treatment of connective tissue lesions is the formation of strong, lasting bonds between the adjacent tissues. For example, tendons and ligaments need to graft to, and integrate with, muscle, bone tissue or other connective tissues.

2. Soft Tissue Implants

A related problem is the correction of soft tissue defects. For example cosmetic implants need to attach to and integrate with the chest wall (breast implants) or the facial bones (cheek, chin, nasal implants); the correction of soft tissue defects also benefits from the formation of strong, lasting bonds between the implant and the underlying tissue. In reconstructive and cosmetic plastic surgery, muscle and soft tissue flaps must be attached to underlying bone or muscle, while facelifts require successful attachment of the superficial facial tissues to the supporting tissues.

Soft tissue implants are used in a variety of cosmetic, plastic, and reconstructive surgical procedures and may be delivered to many different parts of the body, including, without limitation, the face, nose, breast, chin, buttocks, chest, lip and cheek. Soft tissue implants are used for the reconstruction of surgically or traumatically created tissue voids, augmentation of tissues or organs, contouring of tissues, the restoration of bulk to aging tissues, and to correct soft tissue folds or wrinkles (rhytides). Soft tissue implants may be used for the augmentation of tissue for cosmetic (aesthetic) enhancement or in association with reconstructive surgery following disease or surgical resection. Representative examples of soft tissue implants that can be coated with, or otherwise used in combination with the compositions described herein, include, e.g., saline breast implants, silicone breast implants, chin and mandibular implants, nasal implants, cheek implants, lip implants, and other facial implants, pectoral and chest implants, malar and submalar implants, and buttocks implants.

In one aspect, soft tissue implants include or are formed from silicone. Silicone implants can be solid, yet flexible and very durable. They are manufactured in different durometers (degrees of hardness) to be soft or quite hard. These implants are designed to enhance soft tissue areas rather than the underlying bone structure. Silicone implants can be used to augment tissue in a variety of locations in the body, including, for example, cheek, nasal, chin, mid-facial (e.g., cheek), and pectoral area. In certain aspects, silicone-based implants (e.g., chin implants) may be affixed to the underlying bone by way of one or several titanium screws. Using the compositions described in this invention, it is possible to attach silicone facial implants to the underlying bone without the use of screws. In another aspect, soft tissue implants include or are formed from poly(tetrafluoroethylene) (PTFE). In certain aspects, the poly(tetrafluoroethylene) is expanded polytetrafluoroethylene (ePTFE). These implants are porous and can become integrated into the surrounding tissue which aids in maintaining the implant in its appropriate anatomical location. The addition of the compositions described in this invention to the surface of the implant, or infiltrated into the area between the PTFE implant and the underlying bone can further enhance anchorage of the implant. PTFE implants generally are not as firm as silicone implants. Further, there is less bone resorption underneath ePTFE implants as opposed to silicone implants. In yet another aspect, soft tissue implants include or are formed from polyethylene. Polyethylene implants are frequently used, for example in chin augmentation. Polyethylene implants can be porous, such that they may become integrated into the surrounding tissue, which provides an alternative to using titanium screws for stability. In the same manner as described above, utilizing the compositions described in this invention it is possible to attach polyethylene facial implants to the underlying bone without the use of titanium screws.

Examples commercially available polymeric soft tissue implants suitable for use in combination with a fibrosis-inhibitor include silicone implants from Surgiform Technology, Ltd. (Columbia Station, Ohio); ImplantTech Associates (Ventura, Calif.); Inamed Corporation (Santa Barbara, Calif.); Mentor Corporation (Santa Barbara, Calif.); and Allied Biomedical (Ventura, Calif.).

Commercially available breast implant implants suitable for use with the compositions of the present invention include: those from Inamed Corporation (Santa Barbara, Calif.) which sells both Saline-Filled and Silicone-Filled Breast Implants. Inamed's Saline-Filled Breast Implants include the Style 68 Saline Matrix and Style 363LF as well as others in a variety of models, contours, shapes and sizes. Inamed's Silicone-Filled Breast Implants include the Style 10, Style 20 and Style 40 as well as others in a variety of shapes, contours, and sizes. Inamed also sells breast tissue expanders, such as the Inamed Style 133 V series tissue expanders, which are used to encourage rapid tissue adherence to maximize expander immobility. Mentor Corporation (Santa Barbara, Calif.) sells the saline-filled CONTOUR PROFILE® Style Breast Implant (available in a variety of models, shapes, contours and sizes) and the SPECTRUM® Postoperatively Adjustable Breast Implant which allows adjustment of breast size by adding or removing saline with a simple office procedure for six months post-surgery. Mentor also produces the CONTOUR PROFILE® Gel (silicone) breast implant in a variety of models, shapes, contours, and sizes.

Commercially available poly(tetrafluoroethylene) facial implants suitable for use in combination with the compositions of the present invention include poly(tetrafluoroethylene) cheek, chin, and nasal implants from W. L. Gore & Associates, Inc. (Newark, Del.) such as the preformed TRIMENSIONAL® 3-D Implants. Commercially available polyethylene soft tissue implants suitable for use in combination with the compositions of the present invention include polyethylene implants from Porex Surgical Inc. (Newnan, Ga.) sold under the tradename MEDPOR® Biomaterial. MEDPOR® Biomaterial is composed of porous, high-density polyethylene material with an omni-directional latticework of interconnecting pores, which allows for integration into host tissues. Other facial implants suitable for the practice of this invention include: Tissue Technologies, Inc. (San Francisco, Calif.) which sells the ULTRASOFT-RC™ Facial Implant made of soft, pliable synthetic e-PTFE used for soft tissue augmentation of the face. Tissue Technologies, Inc. also sells the ULTRASOFT™ which is made of tubular e-PTFE indicated for soft tissue augmentation of the facial area and is particularly well suited for use in the lip border and the nasolabial folds. A variety of facial implants are available from ImplanTech Associates (Ventura, Calif.) including the BINDER SUBMALA® facial implant, the BINDER SUBMALAR® II Facial Implant, the TERINO MALAR SHELL®, the COMBINED SUBMALAR SHELL™, the FLOWERS TEAR TROUGH™ implant; solid silicone facial and malar implants from Allied Biomedical; the Subcutaneous Augmentation Material (S.A.M.), made from microporous ePTFE.

The compositions described in the present invention can be infiltrated into the space (surgically created pocket) where the breast, facial, or soft tissue implant will be implanted. This can be accomplished by applying the composition: (a) to the breast or facial implant surface during the implantation procedure; (b) to the surface of the tissue of the implantation pocket immediately prior to, or during, implantation of the breast or facial implant; (c) to the surface of the breast or facial implant and/or the tissue surrounding the implant immediately after to the implantation of the soft tissue implant; (d) by topical application of the composition into the anatomical space where the soft tissue implant will be placed; (e) via percutaneous injection into the tissue surrounding the implant; and/or (f) by any combination of the aforementioned methods.

For all soft tissue implants, implant malposition (movement or migration of the implant after placement) can lead to a variety of complications such as asymmetry and is a leading cause of patient dissatisfaction and revision surgery. In particular, facial implants can migrate following surgery and it is important to achieve attachment of the implant to the underlying periosteum and bone tissue. In a preferred embodiment the breast or facial implant is coated on the inferior surface (i.e., the surface facing the pectoralis muscle for subglandular breast implants or the surface facing the chest wall for subpectoral breast implants; or the surface facing the periosteum or bone tissue for facial implants) with the compositions of the present invention. This embodiment has the advantage of encouraging anchorage and fixation of the breast or facial implant into the anatomical location into which it was placed (preventing implant migration). As an alternative to, or in addition to, coating the inferior surface of the breast or facial implant, the compositions can be infiltrated into the space (the base of the surgically created pocket) where the breast or facial implant will be apposed to the underlying tissue.

Administration of the present compositions agent can reduce the incidence of migration, asymmetry and repeat surgical interventions (e.g., revisions and removal of the implants) and improve patient satisfaction.

It should be apparent to one of skill in the art that the same techniques can be applied to the application of the compositions to facial tissues during a facelift or to muscle or skin tissue during placement of a muscle or skin flap. Briefly, autogenous tissue implants may be composed of pedicle flaps that typically originate from the back (e.g., latissimus dorsi myocutaneous flap) or the abdomen (e.g., transverse rectus abdominus myocutaneous or TRAM flap). Pedicle flaps may also come from the buttocks, thigh, or groin. These flaps are detached from the body and then transplanted by reattaching blood vessels using microsurgical procedures. These muscular tissue flaps are most frequently used for post-masectomy closure and reconstruction. Some other common closure applications for muscular tissue flaps include coverage of defects in the head and neck area, especially defects created from major head and neck cancer resection; additional applications include coverage of chest wall defects other than mastectomy deformities. The latissimus dorsi may also be used as a reverse flap, based upon its lumbar perforators, to close congenital defects of the spine such as spina bifida or meningomyelocele. For example, U.S. Pat. No. 5,765,567 to Knowlton describes methodology of using an autogenous tissue implant in the form of a tissue flap having a cutaneous skin island that may be used for contour correction and enlargement for the reconstruction of breast tissue. The tissue flap may be a free flap or a flap attached via a native vascular pedicle. In a preferred embodiment, the autogenous tissue implant is coated on the inferior surface (i.e., the surface facing the attachment site) with the compositions of the present invention. As an alternative to, or in addition to, coating the inferior surface of the autogenous tissue implant, the compositions can be infiltrated into the space (the base of the surgically created pocket) where the autogenous tissue implant will be apposed to the underlying tissue.

A similar method occurs in facelift procedures. In a preferred embodiment, the facial tissues are coated on the inferior surface (i.e., the surface facing the attachment site) with the compositions of the present invention. As an alternative to, or in addition to, coating the inferior surface of the facial tissues, the compositions can be infiltrated into the space (the base of the surgically created pocket) where the facial tissue will be apposed to the underlying tissue.

A facelift may be performed under general anaesthesia or under local anaesthesia. For intravenous sedation, diazepam or midazolam may be used. The amount of drug used should be sufficient to ensure that the patient is in a state of slurred speech but without any compromise of vital functions. Most patients will require 10 mg of valium, but an initial dose of 1 to 2.5 mg is often used. Regardless of anaesthesia selection, 0.5% lidocaine with epinephrine (1:200,000) may be used for local infiltration. Approximately 60 mL of solution is required on one side of the face and neck. In the case of general anesthesia, maintaining a systolic blood pressure between 100 mm Hg and 120 mm Hg is ideal. Attention to the airway is critical given all the facial manipulation involved in the procedure. Facelifts can be performed in the subcutaneous plane or in a deep plane (i.e., the sub-superficial musculoponeurotic system also called “sub-SAMS”). In the case of simple skin laxity, a superficial approach is appropriate. Before the administration of the anesthesia, the planned incisions are marked. Generally, the facelift incision begins in the temporal scalp 5 cm about the ear and 5 cm behind the hairline and curves down parallel to the hairline toward the superior root of the helix and continues caudally in the natural preauricluar skin crease. The incision follows the crus of the helix into the incisura anterior then to the tragus and continues inferiorly in the natural skin crease. After initiation of the dissection with a scalpel, the remaining flap is elevated by scissor dissection and rotation flaps and excessive skin is excised. See, Skoog T., PLASTIC SURGERY—NEW METHODS AND REFINEMENTS (W.B. Saunders, Philadelphia, Pa., 1974). During the procedure, hemostasis is critical and obtained with pricise forceps electrocautery using long insulated forceps. Upon reapproximation of tissue and closure, a very thin layer of CHONDROGEL™ (Angiotech Biomaterials Corp., Palo Alto, Calif.) less than 1 mm in thickness, is sprayed over the tissue before approximation, in order to assure hemostatsis, prevent tissue separation between tissues due to seroma formation, and to facilitate proper approximation or glueing of tissue in place. CHONDROGEL™ is the tradename for a combination of a four-armed thiol PEG (10K), a four-armed NHS-PEG (10K), and methylated collagen. In the case of a patient with more extensive problems, such as submental fat deposits, submandibular fat deposits, or jowls, a SMAS/Platysma facelift is more appropriate. The nature of the platysma surgery is individualized, depending on a number of factors, such as anatomy and amount of submental, submandibular, and subplatysmal fat. The platysma muscle is thin and flat and lies just under the skin. Deep within the platysma muscle lies the superficial layer of the deep cervical fascial; the plane between the two is relatively avascular and easily dissected. After the debulking or alterations are performed, all tissue planes before reapproximation are sprayed with a thin layer of CHONDROGEL™, less than 1 mm in thickness. In a preferred embodiment, the dissected tissues are coated with the compositions of the present invention. As an alternative to, or in addition to, coating the dissected surface of the facial tissues, the compositions can be infiltrated into the space (the base of the surgically created pocket) where the facial tissue will be apposed to the underlying tissue.

3. Application of the Compositions

For all of the indications described above, the repair of damaged tissue may be carried out within the context of any standard surgical process allowing access to and repair of the tissue, including open surgery and arthroscopic techniques. Once the damaged tissue is accessed, the composition of the invention is placed in contact with the damaged tissue along with any surgically acceptable implant (e.g., breast implants, facial implants, autogenous tissue implants, joint replacements, clips, screws, staples, pins, and the like), if needed. When used to repair lacerated or separated tissue, such as by joining two or more tissue surfaces, the composition is applied to one or more of the tissue surfaces and then the surfaces are placed in contact with each other and adhesion occurs therebetween.

Preferably, all reactive components of the composition are first mixed to initiate crosslinking, then delivered to the desired tissue or surface before substantial crosslinking has occurred. The surface or tissue to which the composition containing the components or a partial reaction mixture thereof (i.e., where any two or more components have started to react with each other) has been applied is then contacted with the remaining surface, i.e., another tissue surface or implant surface, preferably immediately, to provide an intermediate layer between the tissues.

Crosslinking is typically sufficiently complete within about 5 to 60 seconds after mixing the components of the composition; however, the time required for complete crosslinking to occur is dependent on a number of factors, including the type and molecular weight of each reactive component, the degree of functionalization, and the concentration of the components in the crosslinkable compositions (e.g., higher component concentrations result in faster crosslinking times).

Alternatively, all reactive components of the composition are first mixed at a pH that does not initiate the reaction, for example, in an aqueuous acidic buffer having a pH of less than 7, then delivered to the desired tissue or surface. Once the composition has its desired shape and consistency, an activating (i.e., initiating) buffer is sprayed on its surface. The initiating buffer, such as a basic buffer (e.g., an aqueous solution having a pH of more than 7) initiates the reaction and crosslinking of the biomaterial. The crosslinking starts immediately and substantial crosslinking occurs within minutes.

### Administration and Use

The compositions of the present invention may be applied to any tissue surface and may be used in any customary method of tissue repair. The composition, as discussed below, is preferably applied before crosslinking of the various components. The compositions of the present invention are generally delivered to the site of administration in such a way that the individual components of the composition come into contact with one another for the first time at the site of administration, or within one hour preceding administration.

Specifically, the compositions may be applied in the form of viscous liquid, partially polymerized gel, suspension, or applied onto the target site by spraying. In one of the embodiments the mixed, but uncrosslinked components of the composition, may be applied in the form of a flow able liquid to the target site, and subsequently the composition may be crosslinked by irrigating the applied composition with a crosslinking-initiating buffer. The compositions may be introduced onto/into the target site by using delivery systems designed to deliver separate liquid reactive components.

Thus, in one embodiment the compositions of the present invention are delivered to the site of administration using an apparatus that allows the components to be delivered separately. Such delivery systems usually involve a multi-compartment spray device. Alternatively, the components can be delivered separately using any type of controllable extrusion system, or they can be delivered manually in the form of separate pastes, liquids or dry powders, and mixed together manually at the site of administration. Many devices that are adapted for delivery of multi-component tissue sealants/hemostatic agents are well known in the art and can also be used in the practice of the present invention.

In another embodiment of the invention, the compositions of the present invention, may be used as a bioadhesive, a biological adhesive, and/or a surgical adhesive to effect temporary or permanent attachment between the surfaces of two native tissues, or between a native tissue surface and either a non-native tissue surface or a surface of a synthetic implant.

Yet another way of delivering the compositions of the present invention is to prepare the reactive components in inactive form as a solution, liquid or powder. Such compositions can then be activated after application to the tissue site, or immediately beforehand, by applying an activator. In one embodiment, the activator is a buffer solution having a pH that will activate the composition once mixed therewith. Still another way of delivering the compositions is to prepare preformed sheets, and apply the sheets as such to the site of administration. One of skill in the art can easily determine the appropriate administration protocol to use with any particular composition having a known gel strength and gelation time. A more detailed description of the composition is given below.

### Compositions for Cartilage and Soft Tissue Repair

The present invention provides an implantable biomaterial for use in the repair of connective tissues such as cartilage, muscle, bone, ligaments and tendons. A related problem is the correction of soft tissue defects including the placement of cosmetic implants (such as breast implants and facial implants), muscle flaps, soft tissue flaps, and lifts (e.g., facelifts, muscle flaps, brow lifts, excess skin removal, tummy tucks, and the like). The described compositions are biocompatible and biodegradable and can promote tissue regeneration at the implantation site into a tissue specific structure (i.e. bone, cartilage, scar tissue, or a ligament-like structure). The characteristics and properties of the composition are chosen to provide for bioresorbability which may stimulate repopulation of the implanted material with tissue specific cells (osteoblasts, chondrocytes, fibroblasts, mesenchimal cells). In one aspect, the compositions are hydrogels which are capable of swelling in the physiological environment at the implantation site in the range between 10% and 200% of their original volume. Hydrogel compositions are structurally resistant to repeated physical forces found at the tissue site, which makes them conducive to the in growth of connective tissue cells from adjacent tissue into the material. Further, the presence of migration and proliferation promoting moieties (e.g., methylated collagen) in the biomaterial matrix may foster remodeling of the implant at the injured site into a functional connective tissue (e.g., hyaline cartilage). Thus, in one aspect, the composition functions as a scaffold that is capable of promoting proliferation, differentiation, and migration of tissue cells adjacent to the material and cells within the material into the phenotypic tissue cells characteristic to for the surrounding tissue.

The composition has a hydrophilic polymer component and a plurality of crosslinkable components. The various components of the composition may crosslink with each other and may additionally crosslink with reactive moieties in the surrounding tissue. Additionally, other components may also be present. A discussion of each of these components is presented below.

### The Hydrophilic Polymer Component

The hydrophilic polymer component may be a synthetic or naturally occurring hydrophilic polymer. Naturally occurring hydrophilic polymers include, but are not limited to: proteins such as collagen and derivatives thereof, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen (e.g., methylated collagen) and glycosaminoglycans are preferred naturally occurring hydrophilic polymers for use herein.

In general, collagen from any source may be used in the composition of the method; for example, collagen may be extracted and purified from human or other mammalian source, such as bovine or porcine corium and human placenta, or may be recombinantly or otherwise produced. The preparation of purified, substantially non-antigenic collagen in solution from bovine skin is well known in the art. Commonly owned U.S. Pat. No. 5,428,022, to Palefsky et al., discloses methods of extracting and purifying collagen from the human placenta. Commonly owned U.S. Pat. No. 5,667,839, to Berg, discloses methods of producing recombinant human collagen in the milk of transgenic animals, including transgenic cows. The term “collagen” or “collagen material” as used herein refers to all forms of collagen, including those that have been processed or otherwise modified.

Collagen of any type, including, but not limited to, types I, II, III, IV, or any combination thereof, may be used in the compositions of the invention, although type I is generally preferred. Either atelopeptide or telopeptide-containing collagen may be used; however, when collagen from a source, such as bovine collagen, is used, atelopeptide collagen is generally preferred, because of its reduced immunogenicity compared to telopeptide-containing collagen.

Collagen that has not been previously crosslinked by methods such as heat, irradiation, or chemical crosslinking agents is preferred for use in the compositions of the invention, although previously crosslinked collagen may be used. Non-crosslinked atelopeptide fibrillar collagen is commercially available from McGhan Medical Corporation (Santa Barbara, Calif.) at collagen concentrations of 35 mg/ml and 65 mg/ml under the trademarks ZYDERM® I Collagen and ZYDERM® II Collagen, respectively. Glutaraldehyde-crosslinked atelopeptide fibrillar collagen is commercially available from Mchan Medical Corporation at a collagen concentration of 35 mg/ml under the trademark ZYPLAST®.

Collagens for use in the present invention are generally, although not necessarily, in aqueous suspension at a concentration between about 20 mg/ml to about 120 mg/ml, preferably between about 30 mg/ml to about 90 mg/ml.

Although intact collagen is preferred, denatured collagen, commonly known as gelatin, can also be used in the compositions of the invention. Gelatin may have the added benefit of being degradable faster than collagen.

Because of its greater surface area and greater concentration of reactive groups, nonfibrillar collagen is generally preferred. The term “nonfibrillar collagen” refers to any modified or unmodified collagen material that is in substantially nonfibrillar form at pH 7, as indicated by optical clarity of an aqueous suspension of the collagen.

Collagen that is already in nonfibrillar form may be used in the compositions of the invention. As used herein, the term “nonfibrillar collagen” is intended to encompass collagen types that are nonfibrillar in native form, as well as collagens that have been chemically modified such that they are in nonfibrillar form at or around neutral pH. Collagen types that are nonfibrillar (or microfibrillar) in native form include types IV, VI, and VII.

Chemically modified collagens that are in nonfibrillar form at neutral pH include succinylated collagen, propylated collagen, ethylated collagen, methylated collagen, and the like, both of which can be prepared according to the methods described in U.S. Pat. No. 4,164,559, to Miyata et al., which is hereby incorporated by reference in its entirety. Due to its inherent tackiness, methylated collagen is particularly preferred, as disclosed in commonly owned U.S. Pat. No. 5,614,587 to Rhee et al.

Collagens for use in the crosslinkable compositions of the present invention may start out in fibrillar form and then become rendered nonfibrillar by the addition of one or more fiber disassembly agents. The fiber disassembly agent must be present in an amount sufficient to render the collagen substantially nonfibrillar at pH 7, as described above. Fiber disassembly agents for use in the present invention include, without limitation, various biocompatible alcohols, amino acids, inorganic salts, and carbohydrates, with biocompatible alcohols being particularly preferred. Preferred biocompatible alcohols include glycerol and propylene glycol. Non-biocompatible alcohols, such as ethanol, methanol, and isopropanol, are not preferred for use in the present invention, due to their potentially deleterious effects on the body of the patient receiving them. Preferred amino acids include arginine. Preferred inorganic salts include sodium chloride and potassium chloride. Although carbohydrates, such as various sugars including sucrose, may be used in the practice of the present invention, they are not as preferred as other types of fiber disassembly agents because they can have cytotoxic effects in vivo.

As fibrillar collagen has less surface area and a lower concentration of reactive groups than nonfibrillar, fibrillar collagen is less preferred; however, as disclosed in commonly owned, U.S. Pat. No. 5,614,587 to Rhee et al. fibrillar collagen, or mixtures of nonfibrillar and fibrillar collagen, may be preferred for use in compositions intended for long-term persistence in vivo, if optical clarity is not a requirement.

Synthetic hydrophilic polymers may also be used in the present invention. Useful synthetic hydrophilic polymers include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di tri-polyoxyethylated glycerol, mono di-polyoxyethylated propylene glycol, and mono di-polyoxyethylated trimethylene glycol; polyoxyethylated sorbitol, polyoxyethylated glucose; acrylic acid polymers and analogues and copolymers thereof such as polyacrylic acid, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

### The Crosslinkable Components

The compositions of the invention also comprise a plurality of crosslinkable components. Each of the crosslinkable components participates in a reaction that results in a crosslinked matrix.

The crosslinkable components are selected so that crosslinking gives rise to a biocompatible, nonimmunogenic matrix useful in a variety of contexts including biologically active agent delivery, tissue augmentation, angiogenesis inhibition, and other applications. The crosslinkable components of the invention comprise: a component A, which has m nucleophilic groups, wherein m≧2 and a component B, which has n electrophilic groups capable of reaction with the m nucleophilic groups, wherein n≧2 and m+n≧4. An optional third component, optional component C, which has at least one functional group that is either electrophilic and capable of reaction with the nucleophilic groups of component A or nucleophilic and capable of reaction with the electrophilic groups of component B may also be present. Thus, the total number of functional groups present on components A, B and C, when present, in combination is ≧5; that is, the total functional groups given by m+n+p must be ≧5, where p is the number of functional groups on component C and, as indicated, is ≧1. Each of the components is biocompatible and nonimmunogenic, and at least one component is comprised of a hydrophilic polymer. Also, as will be appreciated, the composition may contain additional crosslinkable components D, E, F, etc., having one or more reactive nucleophilic or electrophilic groups and thereby participate in formation of the crosslinked biomaterial via covalent bonding to other components.

The m nucleophilic groups on component A may all be the same, or, alternatively, A may contain two or more different nucleophilic groups. Similarly, the n electrophilic groups on component B may all be the same, or two or more different electrophilic groups may be present. The functional group(s) on optional component C, if nucleophilic, may or may not be the same as the nucleophilic groups on component A, and, conversely, if electrophilic, the functional group(s) on optional component C may or may not be the same as the electrophilic groups on component B.

Accordingly, the components may be represented by the structural formulae

R¹(-[Q¹]_{q}-X)ₘ(component A), (I)

R²(-[Q²]ᵣ-Y)ₙ(component B), and (II)

R³(-[Q³]ₛ-Fn)ₚ,(optional component C), (III)

wherein:

R¹, R² and R³ are independently selected from the group consisting of C₂ to C₁₄ hydrocarbyl, heteroatom-containing C₂ to C₁₄ hydrocarbyl, hydrophilic polymers, and hydrophobic polymers, providing that at least one of R¹, R² and R³ is a hydrophilic polymer, preferably a synthetic hydrophilic polymer;

X represents one of the m nucleophilic groups of component A, and the various X moieties on A may be the same or different;

Y represents one of the n electrophilic groups of component B, and the various Y moieties on A may be the same or different;

Fn represents a functional group on optional component C;

Q¹, Q² and Q³ are linking groups;

m≧2, n≧2, m+n is ≧4, q, and r are independently zero or 1, and when optional component C is present, p≧1, and s is independently zero or 1.

### Reactive Groups

X may be virtually any nucleophilic group, so long as reaction can occur with the electrophilic group Y. Analogously, Y may be virtually any electrophilic group, so long as reaction can take place with X. The only limitation is a practical one, in that reaction between X and Y should be fairly rapid and take place automatically upon admixture with an aqueous medium, without need for heat or potentially toxic or non-biodegradable reaction catalysts or other chemical reagents. It is also preferred although not essential that reaction occur without need for ultraviolet or other radiation. Ideally, the reactions between X and Y should be complete in under 60 minutes, preferably under 30 minutes. Most preferably, the reaction occurs in about 5 to 15 minutes or less.

Examples of nucleophilic groups suitable as X include, but are not limited to, —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, —C₅H₄N, etc. wherein R⁴ and R⁵ are hydrocarbyl, typically alkyl or monocyclic aryl, preferably alkyl, and most preferably lower alkyl. Organometallic moieties are also useful nucleophilic groups for the purposes of the invention, particularly those that act as carbanion donors. Organometallic nucleophiles are not, however, preferred. Examples of organometallic moieties include: Grignard functionalities —R⁶MgHal wherein R⁶ is a carbon atom (substituted or unsubstituted), and Hal is halo, typically bromo, iodo or chloro, preferably bromo; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups in the crosslinkable composition, the composition must be admixed with an aqueous base in order to remove a proton and provide an —S⁻ or —O⁻ species to enable reaction with an electrophile. Unless it is desirable for the base to participate in the crosslinking reaction, a nonnucleophilic base is preferred. In some embodiments, the base may be present as a component of a buffer solution. Suitable bases and corresponding crosslinking reactions are described infra in Section E.

The selection of electrophilic groups provided within the crosslinkable composition, i.e., on component B, must be made so that reaction is possible with the specific nucleophilic groups. Thus, when the X moieties are amino groups, the Y groups are selected so as to react with amino groups. Analogously, when the X moieties are sulfhydryl moieties, the corresponding electrophilic groups are sulfhydryl-reactive groups, and the like.

By way of example, when X is amino (generally although not necessarily primary amino), the electrophilic groups present on Y are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyldiimidazole or sulfonyl chloride); and (10) olefins, including conjugated olefins, such as ethanesulfonyl (—SO₂CH═CH₂) and analogous functional groups, including acrylate (—CO₂—C═CH₂), methacrylate (—CO₂—C(CH₃)═CH₂)), ethyl acrylate (—CO₂—C(CH₂CH₃)═CH₂), and ethyleneimino (—CH═CH—C═NH). Since a carboxylic acid group is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxysuccinimide (NHS) or N-hydroxysulfosuccinimide (NHSS) in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when X is sulfhydryl, the electrophilic groups present on Y are groups that react with a sulfhydryl moiety. Such reactive groups include those that form thioester linkages upon reaction with a sulfhydryl group, such as those described in applicants' PCT Pub. No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarimide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-3-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulfhydryl groups. Alternatively, sulfhydryl reactive groups can be employed that form disulfide bonds with sulfhydryl groups; such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, i.e., mild oxidizing agents such as hydrogen peroxide, can be used to facilitate disulfide bond formation.

Yet another class of sulfhydryl reactive groups forms thioether bonds with sulfhydryl groups. Such groups include, inter alia, maleimido, substituted maleimido, haloalkyl, epoxy, imino, and aziridino, as well as olefins (including conjugated olefins) such as ethanesulfonyl, etheneimino, acrylate, methacrylate, and α,β-unsaturated aldehydes and ketones. This class of sulfhydryl reactive groups is particularly preferred as the thioether bonds may provide faster crosslinking and longer in vivo stability.

When X is —OH, the electrophilic functional groups on the remaining component(s) must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When X is an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

The covalent linkages in the crosslinked structure that result upon covalent binding of specific nucleophilic components to specific electrophilic components in the crosslinkable composition include, solely by way of example, the following (the optional linking groups Q¹ and Q² are omitted for clarity):

TABLE 1

| REPRESENTATIVE | | |
|---|---|---|
| NUCLEOPHILIC | | |
| COMPONENT | | |
| (A, optional | REPRESENTATIVE | |
| component C, | ELECTROPHILIC COMPONENT | |
| element FN_{NU}) | (B, FN_{EL}) | RESULTING LINKAGE |
| R¹—NH₂ | R²—O—(CO)—O—N(COCH₂) | R¹—NH—(CO)—O—R² |
| | (succinimidyl carbonate terminus) | |
| R¹—SH | R²—O—(CO)—O—N(COCH₂) | R¹—S—(CO)—O—R² |
| R¹—OH | R²—O—(CO)—O—N(COCH₂) | R¹—O—(CO)—R² |
| R¹—NH₂ | R²—O(CO)—CH═CH₂ | R¹—NH—CH₂CH₂—(CO)—O—R² |
| | (acrylate terminus) | |
| R¹—SH | R²—O—(CO)—CH═CH₂ | R¹—S—CH₂CH₂—(CO)—O—R² |
| R¹—OH | R²—O—(CO)—CH═CH₂ | R¹—O—CH₂CH₂—(CO)—O—R² |
| R¹—NH₂ | R²—O(CO)—(CH₂)₃—CO₂—N(COCH₂) | R¹—NH—(CO)—(CH₂)₃—(CO)—OR² |
| | (succinimidyl glutarate terminus) | |
| R¹—SH | R²—O(CO)—(CH₂)₃—CO₂—N(COCH₂) | R¹—S—(CO)—(CH₂)₃—(CO)—OR² |
| R¹—OH | R²—O(CO)—(CH₂)₃—CO₂—N(COCH₂) | R¹—O—(CO)—(CH₂)₃—(CO)—OR² |
| R¹—NH₂ | R²—O—CH₂—CO₂—N(COCH₂) | R¹—NH—(CO)—CH₂—OR² |
| | (succinimidyl acetate terminus) | |
| R¹—SH | R²—O—CH₂—CO₂—N(COCH₂) | R¹—S—(CO)—CH₂—OR² |
| R¹—OH | R²—O—CH₂—CO₂—N(COCH₂) | R¹—O—(CO)—CH₂—OR² |
| R¹—NH₂ | R²—O—NH(CO)—(CH₂)₂—CO₂—N(COCH₂) | R¹—NH—(CO)—(CH₂)₂—(CO)—NH—OR² |
| | (succinimidyl succinamide terminus) | |
| R¹—SH | R²—O—NH(CO)—(CH₂)₂—CO₂—N(COCH₂) | R¹—S—(CO)—(CH₂)₂—(CO)—NH—OR² |
| R¹—OH | R²—O—NH(CO)—(CH₂)₂—CO₂—N(COCH₂) | R¹—O—(CO)—(CH₂)₂—(CO)—NH—OR² |
| R¹—NH₂ | R²—O—(CH₂)₂—CHO | R¹—NH—(CO)—(CH₂)₂—OR² |
| | (propionaldehyde terminus) | |
| R¹—NH₂ | 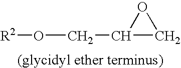 | R¹—NH—CH₂—CH(OH)—CH₂—OR² and ^{t}-N[CH₂—CH(OH)—CH₂—OR²]₂ |
| R¹—NH₂ | R²—O—(CH₂)₂—N═C═O | R¹—NH—(CO)—NH—CH₂—OR² |
| | (isocyanate terminus) | |
| R¹—NH₂ | R²—SO₂—CH═CH₂ | R¹—NH—CH₂CH₂—SO₂—R² |
| | (vinyl sulfone terminus) | |
| R¹—SH | R²—SO₂—CH═CH₂ | R¹—S—CH₂CH₂—SO₂—R² |

### Linking Groups

The functional groups X and Y and FN on optional component C may be directly attached to the compound core (R¹, R² or R³ on optional component C, respectively), or they may be indirectly attached through a linking group, with longer linking groups also termed “chain extenders.” In structural formulae (I), (II) and (III), the optional linking groups are represented by Q¹, Q² and Q³, wherein the linking groups are present when q, r and s are equal to 1 (with R, X, Y, Fn, m n and p as defined previously).

Suitable linking groups are well known in the art. See, for example, PCT Pub. No. WO 97/22371. Linking groups are useful to avoid steric hindrance problems that are sometimes associated with the formation of direct linkages between molecules. Linking groups may additionally be used to link several multifunctionally activated compounds together to make larger molecules. In a preferred embodiment, a linking group can be used to alter the degradative properties of the compositions after administration and resultant gel formation. For example, linking groups can be incorporated into components A, B, or optional component C to promote hydrolysis, to discourage hydrolysis, or to provide a site for enzymatic degradation.

Examples of linking groups that provide hydrolyzable sites, include, inter alia, ester linkages; anhydride linkages, such as obtained by incorporation of glutarate and succinate; ortho ester linkages; ortho carbonate linkages such as trimethylene carbonate; amide linkages; phosphoester linkages; α-hydroxy acid linkages, such as may be obtained by incorporation of lactic acid and glycolic acid; lactone-based linkages, such as may be obtained by incorporation of caprolactone, valerolactone, γ-butyrolactone and p-dioxanone; and amide linkages such as in a dimeric, oligomeric, or poly(amino acid) segment. Examples of non-degradable linking groups include succinimide, propionic acid and carboxymethylate linkages. See, for example, PCT. Pub. No. WO 99/07417. Examples of enzymatically degradable linkages include Leu-Gly-Pro-Ala, which is degraded by collagenase; and Gly-Pro-Lys, which is degraded by plasmin.

Linking groups can also enhance or suppress the reactivity of the various nucleophilic and electrophilic groups. For example, electron-withdrawing groups within one or two carbons of a sulfhydryl group would be expected to diminish its effectiveness in coupling, due to a lowering of nucleophilicity. Carbon-carbon double bonds and carbonyl groups will also have such an effect. Conversely, electron-withdrawing groups adjacent to a carbonyl group (e.g., the reactive carbonyl of glutaryl-N-hydroxysuccinimidyl) would increase the reactivity of the carbonyl carbon with respect to an incoming nucleophile. By contrast, sterically bulky groups in the vicinity of a functional group can be used to diminish reactivity and thus coupling rate as a result of steric hindrance.

By way of example, particular linking groups and corresponding component structure are indicated in Table 2:

TABLE 2

| LINKING GROUP | COMPONENT STRUCTURE |
|---|---|
| —O—(CH₂)ₙ— | Component A: R¹—O—(CH₂)ₙ—X |
| | Component B: R²—O—(CH₂)ₙ—Y |
| | Optional Component C: R³—O—(CH₂)ₙ—Z |
| —S—(CH₂)ₙ— | Component A: R¹—S—(CH₂)ₙ—X |
| | Component B: R²—S—(CH₂)ₙ—Y |
| | Optional Component C: R³—S—(CH₂)ₙ—Z |
| —NH—(CH₂)ₙ— | Component A: R¹—NH—(CH₂)ₙ—X |
| | Component B: R²—NH—(CH₂)ₙ—Y |
| | Optional Component C: R³—NH—(CH₂)ₙ—Z |
| —O—(CO)—NH—(CH₂)ₙ— | Component A: R¹—O—(CO)—NH—(CH₂)ₙ—X |
| | Component B: R²—O—(CO)—NH—(CH₂)ₙ—Y |
| | Optional Component C: R³—O—(CO)—NH—(CH₂)ₙ—Z |
| —NH—(CO)—O—(CH₂)ₙ— | Component A: R¹—NH—(CO)—O—(CH₂)ₙ—X |
| | Component B: R²—NH—(CO)—O—(CH₂)ₙ—Y |
| | Optional Component C: R³—NH—(CO)—O—(CH₂)ₙ—Z |
| —O—(CO)—(CH₂)ₙ— | Component A: R¹—O—(CO)—(CH₂)ₙ—X |
| | Component B: R²—O—(CO)—(CH₂)ₙ—Y |
| | Optional Component C: R³—O—(CO)—(CH₂)ₙ—Z |
| —(CO)—O—(CH₂)ₙ— | Component A: R¹—(CO)—O—(CH₂)ₙ—X |
| | Component B: R²—(CO)—O—(CH₂)ₙ—Y |
| | Optional Component C: R³—(CO)—O—(CH₂)ₙ—Z |
| —O—(CO)—O—(CH₂)ₙ— | Component A: R¹—O—(CO)—O—(CH₂)ₙ—X |
| | Component B: R²—O—(CO)—O—(CH₂)ₙ—Y |
| | Optional Component C: R³—O—(CO)—O—(CH₂)ₙ—Z |
| —O—(CO)—CHR⁷— | Component A: R¹—O—(CO)—CHR⁷—X |
| | Component B: R²—O—(CO)—CHR⁷—Y |
| | Optional Component C: R³—O—(CO)—CHR⁷—Z |
| —O—R⁸—(CO)—NH— | Component A: R¹—O—R⁸—(CO)—NH—X |
| | Component B: R²—O—R⁸—(CO)—NH—Y |
| | Optional Component C: R³—O—R⁸—(CO)—NH—Z |

In Table 2, n is generally in the range of 1 to about 10, R⁷ is generally hydrocarbyl, typically alkyl or aryl, preferably alkyl, and most preferably lower alkyl, and R⁸ is hydrocarbylene, heteroatom-containing hydrocarbylene, substituted hydrocarbylene, or substituted heteroatom-containing hydrocarbylene) typically alkylene or arylene (again, optionally substituted and/or containing a heteroatom), preferably lower alkylene (e.g., methylene, ethylene, n-propylene, n-butylene, etc.), phenylene, or amidoalkylene (e.g., —(CO)—NH—CH₂).

Other general principles that should be considered with respect to linking groups are as follows: If higher molecular weight components are to be used, they preferably have biodegradable linkages as described above, so that fragments larger than 20,000 MW are not generated during resorption in the body. In addition, to promote water miscibility and/or solubility, it may be desired to add sufficient electric charge or hydrophilicity. Hydrophilic groups can be easily introduced using known chemical synthesis, so long as they do not give rise to unwanted swelling or an undesirable decrease in compressive strength. In particular, polyalkoxy segments may weaken gel strength.

### The Component Core

The “core” of each crosslinkable component is comprised of the molecular structure to which the nucleophilic or electrophilic groups are bound. Using the formulae (I) R¹-[Q¹]_{q}-X)ₘ, for component A, (II) R²(-[Q²]ᵣ-Y)ₙ for component B, and (III) R³(-[Q³]ₛ-Fn)ₚ for optional component C, the “core” groups are R¹, R² and R³. Each molecular core of the reactive components of the crosslinkable composition is generally selected from synthetic and naturally occurring hydrophilic polymers, hydrophobic polymers, and C₂-C₁₄ hydrocarbyl groups zero to two heteroatoms selected from N, O and S, with the proviso that at least one of the crosslinkable components A, B, and optionally C, comprises a molecular core of a synthetic hydrophilic polymer. In a preferred embodiment, at least one of A and B comprises a molecular core of a synthetic hydrophilic polymer.

### Hydrophilic Polymers and “Activation” Thereof

A “hydrophilic polymer” as used herein refers to a synthetic polymer having an average molecular weight and composition effective to render the polymer “hydrophilic” as defined above. As discussed above, synthetic hydrophilic polymers useful herein include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di tri-polyoxyethylated glycerol, mono di-polyoxyethylated propylene glycol, and mono di-polyoxyethylated trimethylene glycol; polyoxyethylated sorbitol, polyoxyethylated glucose; acrylic acid polymers and analogue, and copolymers thereof, such as polyacrylic acid, polymethacrylic acid, poly(hydroxyethyl-methacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof; polyoxazolines, including poly(methyloxazoline) and poly(ethyloxazoline); and polyvinylamines. It must be emphasized that the aforementioned list of polymers is not exhaustive, and a variety of other synthetic hydrophilic polymers may be used, as will be appreciated by those skilled in the art.

The synthetic hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Other suitable synthetic hydrophilic polymers include chemically synthesized polypeptides, particularly polynucleophilic polypeptides that have been synthesized to incorporate amino acids containing primary amino groups (such as lysine) and/or amino acids containing thiol groups (such as cysteine). Poly(lysine), a synthetically produced polymer of the amino acid lysine (145 MW), is particularly preferred. Poly(lysine)s have been prepared having anywhere from 6 to about 4,000 primary amino groups, corresponding to molecular weights of about 870 to about 580,000. Poly(lysine)s for use in the present invention preferably have a molecular weight within the range of about 1,000 to about 300,000, more preferably within the range of about 5,000 to about 100,000, and most preferably, within the range of about 8,000 to about 15,000. Poly(lysine)s of varying molecular weights are commercially available from Peninsula Laboratories Inc. (Belmont, Calif.).

The synthetic hydrophilic polymer may be a homopolymer, a block copolymer, a random copolymer, or a graft copolymer. In addition, the polymer may be linear or branched, and if branched, may be minimally to highly branched, dendrimeric, hyperbranched, or a star polymer. The polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

Although a variety of different synthetic hydrophilic polymers can be used in the present compositions, as indicated above, preferred synthetic hydrophilic polymers are polyethylene glycol (PEG) and polyglycerol (PG), particularly highly branched polyglycerol. Various forms of PEG are extensively used in the modification of biologically active molecules because PEG lacks toxicity, antigenicity, and immunogenicity (i.e., is biocompatible), can be formulated so as to have a wide range of solubilities, and does not typically interfere with the enzymatic activities and/or conformations of peptides. A particularly preferred synthetic hydrophilic polymer for certain applications is a polyethylene glycol (PEG) having a molecular weight within the range of about 100 to about 100,000 MW, although for highly branched PEG, far higher molecular weight polymers can be employed—up to 1,000,000 or more—providing that biodegradable sites are incorporated ensuring that all degradation products will have a molecular weight of less than about 30,000. For most PEGs, however, the preferred molecular weight is about 1,000 to about 20,000 MW, more preferably within the range of about 7,500 to about 20,000 MW Most preferably, the polyethylene glycol has a molecular weight of approximately 10,000 MW

Naturally occurring hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives. Collagen and glycosaminoglycans are examples of naturally occurring hydrophilic polymers for use herein, with methylated collagen being a preferred hydrophilic polymer.

Any of the hydrophilic polymers herein must contain, or be activated to contain, functional groups, i.e., nucleophilic or electrophilic groups, which enable crosslinking. Activation of PEG is discussed below; it is to be understood, however, that the following discussion is for purposes of illustration and analogous techniques may be employed with other polymers.

With respect to PEG, first of all, various functionalized polyethylene glycols have been used effectively in fields such as protein modification (see, Abuchowski et al., ENZYMES AS DRUGS, pp. 367-383 (John Wiley & Sons, New York, N.Y. 1981); and Dreborg et al., CRIT. REV. THERAP. DRUG CARRIER SYST. 6:315 (1990)), peptide chemistry (see, Mutter et al., THE PEPTIDES 2:285-332 (Academic Press, New York, N.Y.), and Zalipsky et al., INT. J. PEPTIDE PROTEIN RES. 30:740 (1987)), and the synthesis of polymeric drugs (see, Zalipsky et al., FUR. POLYM. J. 19:1177 (1983); and Ouchi et al., J. MACROMOL. SCI. CHEM. A24:1011 (1987)).

Activated forms of PEG, including multifunctionally activated PEG, are commercially available, and are also easily prepared using known methods. For example, see Chapter 22 of POLY (ETHYLENE GLYCOL) CHEMISTRY: BIOTECHNICAL AND BIOMEDICAL APPLICATIONS (J. Milton Harris, ed., Plenum Press, NY 1992); and Shearwater Polymers, Inc. Catalog, POLYETHYLENE GLYCOL DERIVATIVES, Huntsville, Ala. (1997-1998).

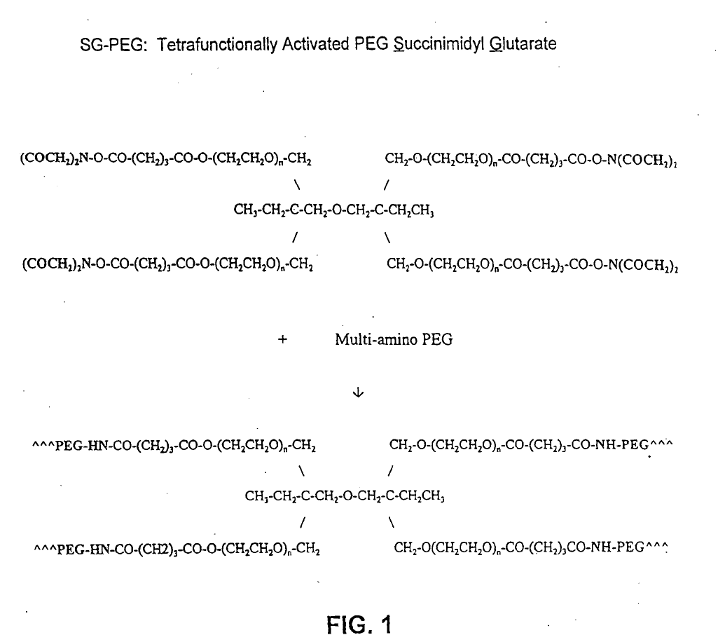

Structures for some specific, tetrafunctionally-activated forms of PEG are shown in FIGS. 1 to 10, as are generalized reaction products that are obtained through reaction of the activated PEGs with multi-amino PEGs, i.e., a PEG with two or more primary amino groups. The activated PEGs illustrated have a pentaerythritol (2,2-bis(hydroxymethyl)-1,3-propanediol) core. Such activated PEGs, as will be appreciated by those in the art, are readily prepared by conversion of the exposed hydroxyl groups in the PEGylated polyol (i.e., the terminal hydroxyl groups on the PEG chains) to carboxylic acid groups (typically by reaction with an anhydride in the presence of a nitrogenous base), followed by esterification with N-hydroxysuccinimide, N-hydroxysulfosuccinimide, or the like, to give the polyfunctionally activated PEG.

FIG. 1 shows the reaction of tetrafunctionally activated PEG succinimidyl glutarate, referred to herein as “SG-PEG,” with multi-amino PEG, and the reaction product obtained thereby.

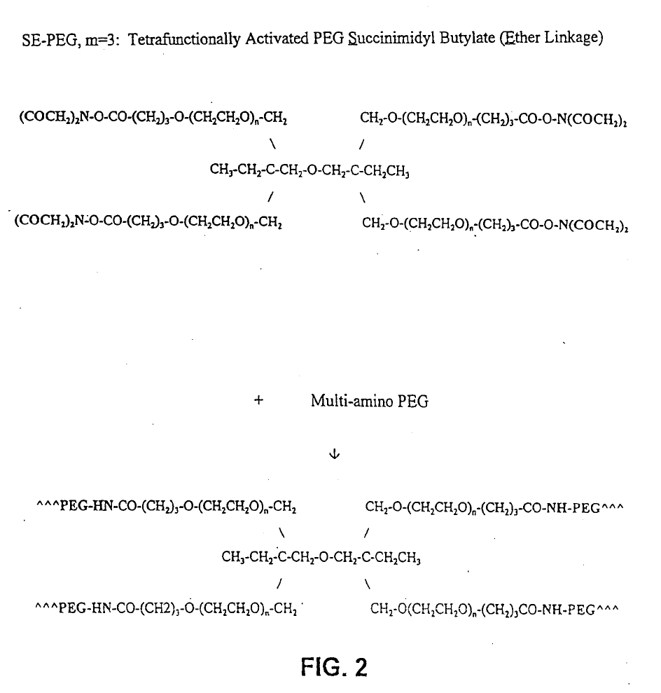

Another activated form of PEG is PEG succinimidyl propionate (“SE-PEG”). The structural formula for tetrafunctionally activated SE-PEG and the reaction product obtained upon reaction with multi-amino PEG are shown in FIG. 2.

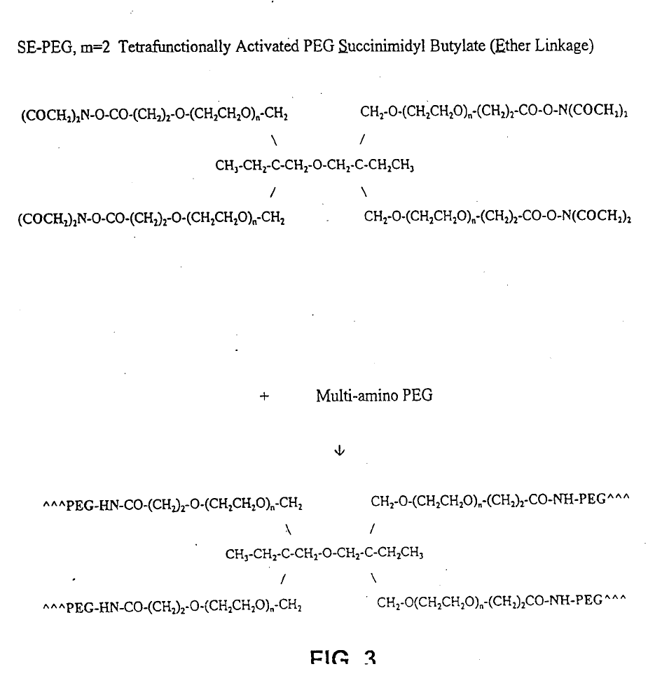

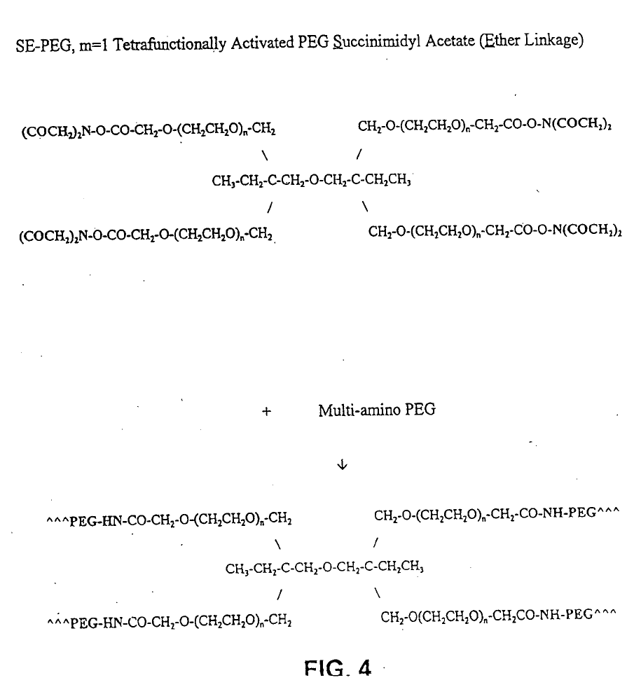

Analogous activated forms of PEG are PEG succinimidyl butylate and PEG succinimidyl acetate, the structures of which are shown in FIGS. 3 and 4, respectively, along with the reaction products obtained upon reaction with multi-amino PEG. SE-PEG, PEG succinimidyl butylate, and PEG succinimidyl acetate are sometimes referred to as “PEG succinimidyl” (PEG-S); see U.S. Pat. No. 5,328,955 to Rhee et at.

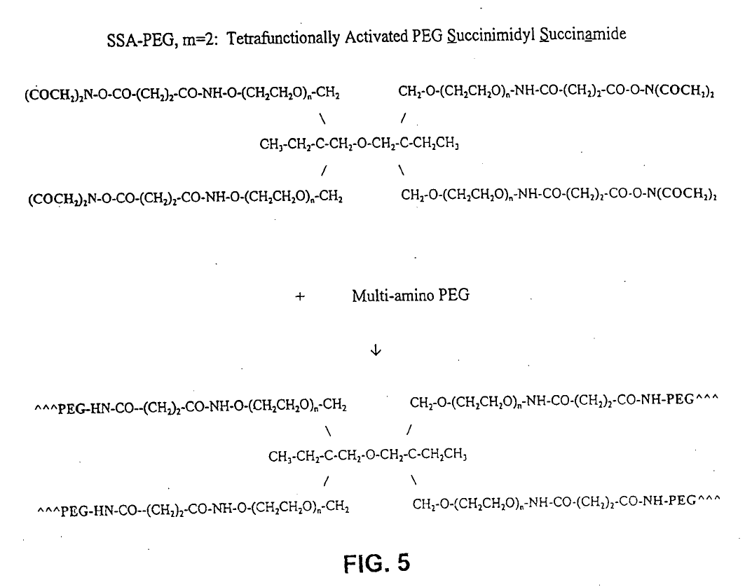

Another functionally activated form of PEG is referred to as “PEG succinimidyl succinamide” (SSA-PEG). The structural formula for the tetrafunctionally activated form of this compound and the reaction product obtained by reacting it with multi-amino PEG are shown in FIG. 5. In the structure of FIG. 5, an ethylene (—CH₂CH₂—) group is shown adjacent to the succinimidyl ester; it is to be understood, however, that as with the PEG succinimidyl compounds, related structures containing a methylene linkage, an n-propylene linkage, or the like, are also possible.

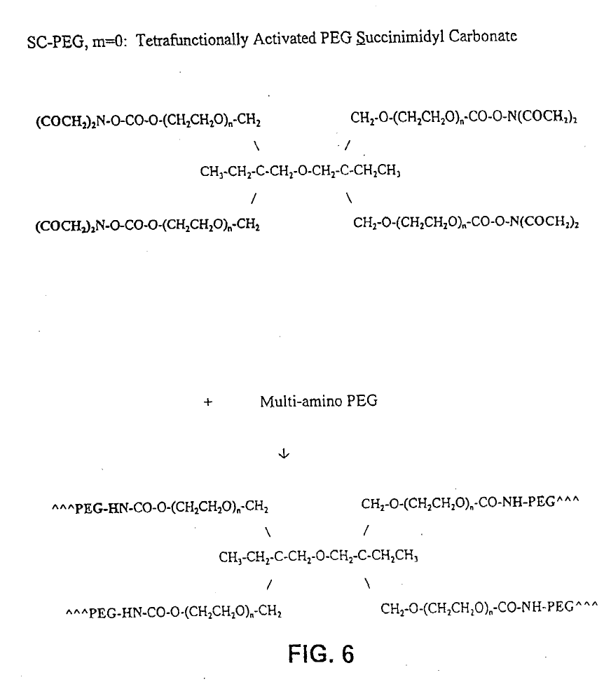

Yet another activated form of PEG is PEG succinimidyl carbonate (SC-PEG). The structural formula of tetrafunctionally activated SC-PEG and the conjugate formed by reacting it with multi-amino PEG are shown in FIG. 6.

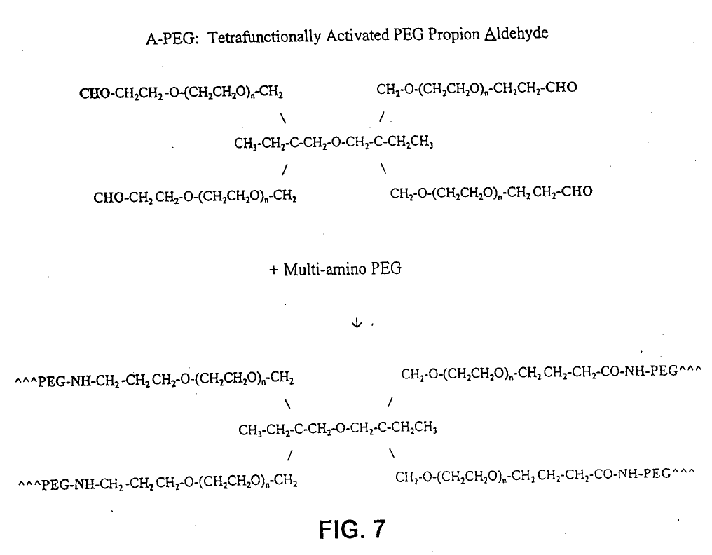

PEG can also be derivatized to form functionally activated PEG propionaldehyde (A-PEG), the tetrafunctionally activated form of which is shown in FIG. 7, as is the conjugate formed by the reaction of A-PEG with multi-amino PEG.

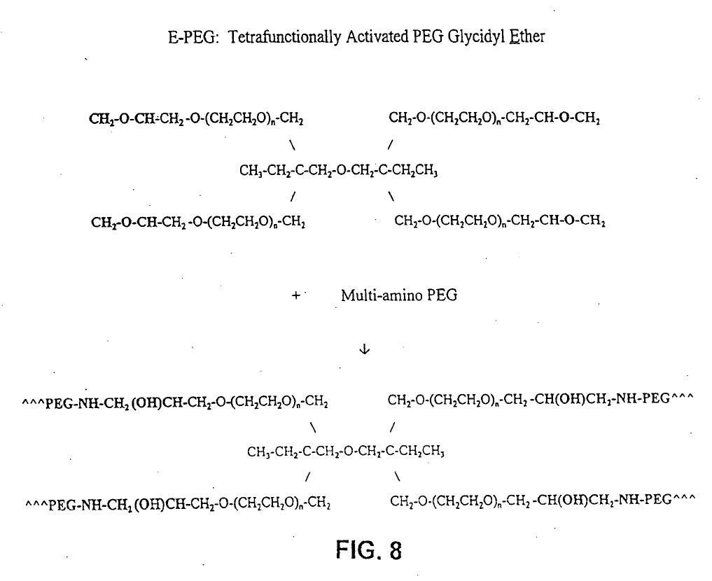

Yet another form of activated polyethylene glycol is functionally activated PEG glycidyl ether (E-PEG), of which the tetrafunctionally activated compound is shown in FIG. 8, as is the conjugate formed by reacting such with multi-amino PEG.

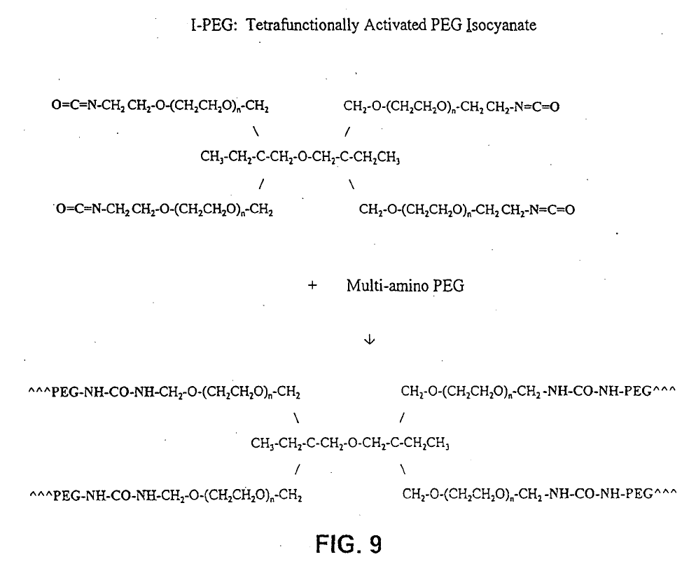

Another activated derivative of polyethylene glycol is functionally activated PEG-isocyanate (I-PEG), which is shown in FIG. 9, along with the conjugate formed by reacting such with multi-amino PEG.

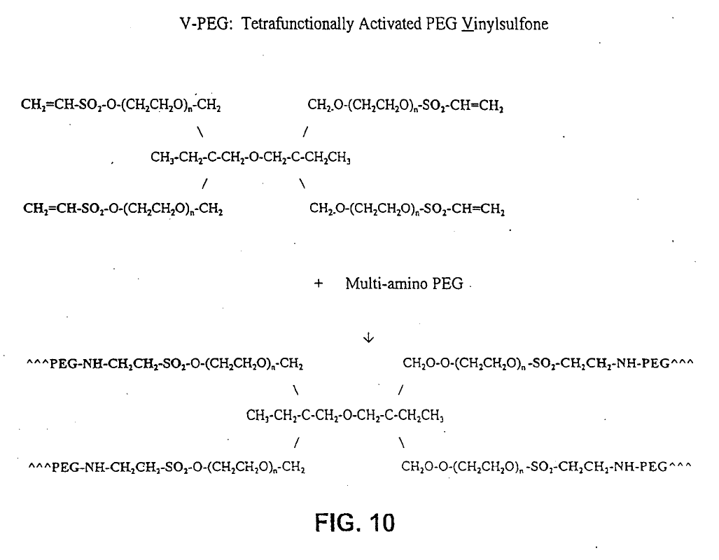

Another activated derivative of polyethylene glycol is functionally activated PEG-vinylsulfone (V-PEG), which is shown in FIG. 10, along with the conjugate formed by reacting such with multi-amino PEG.

Activation with succinimidyl groups to convert terminal carboxylic acid groups to reactive esters is one technique for preparing a synthetic hydrophilic polymer with electrophilic moieties suitable for reaction with nucleophiles such as primary amines, thiols, and hydroxyl groups. Other activating agents for hydroxyl groups include carbonyldiimidazole and sulfonyl chloride; however, as discussed in part (B) of this section, a wide variety of electrophilic groups may be advantageously employed for reaction with corresponding nucleophiles. Examples of such electrophilic groups include acid chloride groups; anhydrides, ketones, aldehydes, isocyanate, isothiocyanate, epoxides, and olefins, including conjugated olefins such as ethanesulfonyl(—SO₂CH═CH₂) and analogous functional groups.

Hydrophilic di- or poly-nucleophilic polymers of the present composition are exemplified in FIGS. 1-10 by multi-amino PEG. Various forms of multi-amino PEG are commercially available from Shearwater Polymers (Huntsville, Ala.) and from Texaco Chemical Company (Houston, Tex.) under the name “Jeffamine.” Multi-amino PEGs useful in the present invention include Texaco's Jeffamine diamines (“D” series) and triamines (“T” series), which contain two and three primary amino groups per molecule. Analogous poly(sulfhydryl) PEGs are also available from Shearwater Polymers, e.g., in the form of pentaerythritol poly(ethylene glycol) ether tetra-sulfhydryl (molecular weight 10,000).

### Hydrophobic Polymers

The crosslinkable compositions of the invention can also include hydrophobic polymers, although for most uses hydrophilic polymers are preferred. Polylactic acid and polyglycolic acid are examples of two hydrophobic polymers that can be used. With other hydrophobic polymers, only short-chain oligomers should be used, containing at most about 14 carbon atoms, to avoid solubility-related problems during reaction.

### Low Molecular Weight Components

As indicated above, the molecular core of one or more of the crosslinkable components can also be a low molecular weight compound, i.e., a C₂-C₁₄ hydrocarbyl group containing zero to 2 heteroatoms selected from N, O, S, and combinations thereof. Such a molecular core can be substituted with nucleophilic groups or with electrophilic groups.

When the low molecular weight molecular core is substituted with primary amino groups, the component may be, for example, ethylenediamine (H₂N—CH₂CH₂—NH₂), tetramethylenediamine (H₂N—(CH₄)—NH₂), pentamethylenediamine (cadaverine) (H₂N—(CH₅)—NH₂), hexamethylenediamine (H₂N—(CH₆)—NH₂), bis(2-aminoethyl)amine (HN—[CH₂CH₂—NH₂]₂), or tris(2-aminoethyl)amine (N—[CH₂CH₂—NH₂]₃).

Low molecular weight diols and polyols include trimethylolpropane, di(trimethylol propane), pentaerythritol, and diglycerol, all of which require activation with a base in order to facilitate their reaction as nucleophiles. Such diols and polyols may also be functionalized to provide di poly-carboxylic acids, functional groups that are, as noted earlier herein, also useful as nucleophiles under certain conditions. Polyacids for use in the present compositions include, without limitation, trimethylolpropane-based tricarboxylic acid, di(trimethylol propane)-based tetracarboxylic acid, heptanedioic acid, octanedioic acid (suberic acid), and hexadecanedioic acid (thapsic acid), all of which are commercially available and/or readily synthesized using known techniques.

Low molecular weight di poly-electrophiles include, for example, disuccinimidyl suberate (DSS), bis(sulfosuccinimidyl) suberate (BS₃), dithiobis(succinimidylpropionate) (DSP), bis(2-succinimidooxycarbonyloxy)ethyl sulfone (BSOCOES), and 3,3′-dithiobis(sulfosuccinimidylpropionate (DTSPP), and their analogues and derivatives. The aforementioned compounds are commercially available from Pierce (Rockford, Ill.). Such di poly-electrophiles can also be synthesized from di polyacids, for example by reaction with an appropriate molar amount of N-hydroxysuccinimide in the presence of DCC. Polyols such as trimethylolpropane and di(trimethylol propane) can be converted to carboxylic acid form using various known techniques, then further derivatized by reaction with NHS in the presence of DCC to produce trifunctionally and tetrafunctionally activated polymers.

### Compositions for the Delivery of Therapeutic Agents

The compositions of the present invention may be used for the delivery of desired therapeutic agents. The therapeutic agents can be delivered, for example, by polymeric carriers, which are discussed in further detail below. The therapeutic agents may be admixed with, blended with, conjugated to, or, otherwise modified to be contained within the compositions of the present invention, which may include biodegradable or non-biodegradable hydrophilic or hydrophobic polymers. Such therapeutic agents may include, for example, fibrosis-inhibiting agents and anti-angiogenic factors.

Representative examples of biodegradable polymers suitable for the delivery of therapeutic agents include albumin, collagen, gelatin, hyaluronic acid, starch, cellulose and cellulose derivatives (e.g., regenerated cellulose, methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(ether ester) multiblock copolymers, based on poly(ethylene glycol) and poly(butylene terephthalate), tyrosine-derived polycarbonates (e.g., U.S. Pat. No. 6,120,491), poly(hydroxyl acids), poly(D,L-lactide), poly(D,L-lactide-co-glycolide), poly(glycolide), poly(hydroxybutyrate), polydioxanone, poly(alkylcarbonate) and poly(orthoesters), polyesters, poly(hydroxyvaleric acid), polydioxanone, polyesters, poly(malic acid), poly(tartronic acid), poly(acrylamides), polyanhydrides, polyphosphazenes, poly(amino acids), poly(alkylene oxide)-poly(ester) block copolymers (e.g., X-Y, X-Y-X, Y-X-Y, R-(Y-X)n, or R-(X-Y)n, where X is a polyalkylene oxide (e.g., poly(ethylene glycol, poly(propylene glycol) and block copolymers of poly(ethylene oxide) and poly(propylene oxide) (e.g., PLURONIC® and PLURONIC® R series of polymers from BASF Corporation, Mount Olive, N.J.) and Y is a polyester, where the polyester may comprise the residues of one or more of the monomers selected from lactide, lactic acid, glycolide, glycolic acid, ε-caprolactone, γ-caprolactone, hydroxyvaleric acid, hydroxybutyric acid, beta-butyrolactone, γ-butyrolactone, γ-valerolactone, γ-decanolactone, δ-decanolactone, trimethylene carbonate, 1,4-dioxane-2-one or 1,5-dioxepan-2one (e.g., PLGA, PLA, PCL, polydioxanone and copolymers thereof) and R is a multifunctional initiator), and the copolymers as well as blends thereof. See e.g., Illum, L., Davids, S. S. (eds.) POLYMERS IN CONTROLLED DRUG DELIVERY (Wright, Bristol 1987); Arshady, J., CONTROLLED RELEASE 17:1-22 (1991); Pitt, INT. J. PHAR. 59:173-196 (1990); Holland et al., J. CONTROLLED RELEASE 4:155-0180 (1986).

Representative examples of non-degradable polymers suitable for the delivery of the aforementioned therapeutic agents include poly(ethylene-co-vinyl acetate) (EVA) copolymers, aromatic polyesters, such as poly(ethylene terephthalate), silicone rubber, acrylic polymers (polyacrylate, polyacrylic acid, polymethylacrylic acid, polymethylmethacrylate, poly(butyl methacrylate)), poly(alkylcynoacrylate) (e.g., poly(ethylcyanoacrylate), poly(butylcyanoacrylate) poly(hexylcyanoacrylate) poly(octylcyanoacrylate)), acrylic resin, polyethylene, polypropylene, polyamides (nylon 6,6), polyurethanes (e.g., CHRONOFLEX® AL and CHRONOFLEX® AR (both from CardioTech International, Inc., Woburn, Mass.), TECOFLEX®, and BIONATE® (Polymer Technology Group, Inc., Emeryville, Calif.), poly(ester urethanes), poly(ether urethanes), poly(ester-urea), polyethers (poly(ethylene oxide), poly(propylene oxide), polyoxyalkylene ether block copolymers based on ethylene oxide and propylene oxide such as the PLURONIC® polymers (e.g., F-127 or F-87) and poly(tetramethylene glycol), styrene-based polymers (polystyrene, poly(styrene sulfonic acid), poly(styrene)-block-poly(isobutylene)-block-poly(styrene), poly(styrene)-poly(isoprene) block copolymers), and vinyl polymers (polyvinylpyrrolidone, poly(vinyl alcohol), poly(vinyl acetate phthalate) as well as copolymers and blends thereof. Polymers may also be developed which are either anionic (e.g., alginate, carrageenan, carboxymethyl cellulose, poly(acrylamido-2-methyl propane sulfonic acid) and copolymers thereof, poly(methacrylic acid and copolymers thereof and poly(acrylic acid) and copolymers thereof, as well as blends thereof, or cationic (e.g., chitosan, poly-L-lysine, polyethylenimine, and poly(allyl amine)) and blends thereof. See e.g., Dunn et al., J. APPLIED POLYMER SCI. 50:353-365 (1993); Cascone et al., J. MATERIALS SCI.: MATERIALS 1N MEDICINE 5:770-774 (1994); Shiraishi et al., BIOL. PHARM. BULL. 16(11):1164-1168 (1993); Thacharodi and Rao, INT'L J. PHARM. 120:115-118 (1995); Miyazaki et al., INT'L J. PHARM. 18:257-263 (1995).

Some examples of preferred polymeric carriers for use with the compositions of the present invention for the delivery of therapeutic agents include poly(ethylene-co-vinyl acetate), polyurethanes, poly (D,L-lactic acid) oligomers and polymers, poly (L-lactic acid) oligomers and polymers, poly (glycolic acid), copolymers of lactic acid and glycolic acid, copolymers of lactide and glycolide, poly (caprolactone), poly (valerolactone), polyanhydrides, copolymers of poly (caprolactone) or poly (lactic acid) with a polyethylene glycol (e.g., MePEG), block copolymers of the form X-Y, X-Y-X, Y-X-Y, R—(Y-X)n, or R-(X-Y)n, where X is a polyalkylene oxide (e.g., poly(ethylene glycol, poly(propylene glycol) and block copolymers of poly(ethylene oxide) and poly(propylene oxide) (e.g., PLURONIC® and PLURONIC® R series of polymers) and Y is a polyester, where the polyester may comprise the residues of one or more of the monomers selected from lactide, lactic acid, glycolide, glycolic acid, ε-caprolactone, γ-caprolactone, hydroxyvaleric acid, hydroxybutyric acid, beta-butyrolactone, γ-butyrolactone, γ-valerolactone, γ-decanolactone, δ-decanolactone, trimethylene carbonate, 1,4-dioxane-2-one or 1,5-dioxepan-2one and R is a multifunctional initiator), silicone rubbers, poly(styrene)block-poly(isobutylene)-block-poly(styrene), poly(acrylate) polymers and blends, admixtures, or co-polymers of any of the above. Other preferred polymers include collagen, poly(alkylene oxide)-based polymers, polysaccharides such as hyaluronic acid, chitosan and fucans, and copolymers of polysaccharides with degradable polymers.

Other representative polymers capable of sustained localized delivery therapeutic agents include carboxylic polymers, polyacetates, polycarbonates, polyethers, polyethylenes, polyvinylbutyrals, polysilanes, polyureas, polyoxides, polystyrenes, polysulfides, polysulfones, polysulfonides, polyvinylhalides, pyrrolidones, rubbers, thermal-setting polymers, cross-linkable acrylic and methacrylic polymers, ethylene acrylic acid copolymers, styrene acrylic copolymers, vinyl acetate polymers and copolymers, vinyl acetal polymers and copolymers, epoxies, melamines, other amino resins, phenolic polymers, and copolymers thereof, water-insoluble cellulose ester polymers (including cellulose acetate propionate, cellulose acetate, cellulose acetate butyrate, cellulose nitrate, cellulose acetate phthalate, and mixtures thereof), polyvinylpyrrolidone, polyethylene glycols, polyethylene oxide, polyvinyl alcohol, potyethers, polysaccharides, hydrophilic polyurethane, polyhydroxyacrylate, dextran, xanthan, hydroxypropyl cellulose, and homopolymers and copolymers of N-vinylpyrrolidone, N-vinyllactam, N-vinyl butyrolactam, N-vinyl caprolactam, other vinyl compounds having polar pendant groups, acrylate and methacrylate having hydrophilic esterifying groups, hydroxyacrylate, and acrylic acid, and combinations thereof; cellulose esters and ethers, ethyl cellulose, hydroxyethyl cellulose, cellulose nitrate, cellulose acetate, cellulose acetate butyrate, cellulose acetate propionate, natural and synthetic elastomers, rubber, acetal, styrene polybutadiene, acrylic resin, polyvinylidene chloride, polycarbonate, homopolymers and copolymers of vinyl compounds, polyvinylchloride, and polyvinylchloride acetate.

Representative examples of patents and publications relating to drug-delivery polymers and their preparation include PCT Pub. Nos. WO 98/19713, WO 01/17575, WO 01/41821, WO 01/41822, and WO 01/15526 (as well as the corresponding U.S. applications), U.S. Pat. Nos. 4,500,676; 4,582,865; 4,629,623; 4,636,524; 4,713,448; 4,795,741; 4,913,743; 5,069,899; 5,099,013; 5,128,326; 5,143,724; 5,153,174; 5,246,698; 5,266,563; 5,399,351; 5,525,348; 5,800,412; 5,837,226; 5,942,555; 5,997,517; 6,007,833; 6,071,447; 6,090,995; 6,106,473; 6,110,483; 6,121,027; 6,156,345; 6,214,901; 6,368,611; 6,630,155; 6,528,080; RE37,950; 6,46,1631; 6,143,314; 5,990,194; 5,792,469; 5,780,044; 5,759,563; 5,744,153; 5,739,176; 5,733,950; 5,681,873; 5,599,552; 5,340,849; 5,278,202; 5,278,201; 6,589,549; 6,287,588; 6,201,072; 6,117,949; 6,004,573; 5,702,717; 6,413,539; 5,714,159; 5,612,052, and U.S. Patent App. Pub. Nos. 2003/0068377, 2002/0192286, 2002/0076441, and 2002/0090398.

It should be obvious to one of skill in the art that the polymers as described herein can also be blended or copolymerized in various compositions as required to deliver therapeutic doses of biologically active agents.

Polymeric carriers can be fashioned in a variety of forms, with desired release characteristics and/or with specific properties depending upon the composition being utilized. For example, polymeric carriers may be fashioned to release a therapeutic agent upon exposure to a specific triggering event such as pH (see e.g., Heller et al., Chemically Self-Regulated Drug Delivery Systems in POLYMERS IN MEDICINE III 175-188 (Elsevier Science Publishers B. V., Amsterdam, 1988); Kang et al., J. APPLIED POLYMER SCI. 48:343-354 (1993); Dong et al., J. CONTROLLED RELEASE 19:171-178 (1992); Dong and Hoffman, J. CONTROLLED RELEASE 15:141-152 (1991); Kim et al., J. CONTROLLED RELEASE 28:143-152 (1994); Cornejo-Bravo et al., J. CONTROLLED RELEASE 33:223-229 (1995); Wu and Lee, PHARM. RES. 10(10): 1544-1547 (1993); Serres et al., PHARM. RES. 13(2):196-201 (1996); Peppas, Fundamentals of pH-and Temperature-Sensitive Delivery Systems, in PULSATILE DRUG DELIVERY 41-55 (Gurny et al. eds. Wissenschaftliche Verlagsgesellschaft mbH, Stuttgart, 1993); Doelker, Cellulose Derivatives in BIOPOLYMERS I (Peppas and Langer, eds., Springer-Verlag, Berlin 1993,). Representative examples of pH-sensitive polymers include poly (acrylic acid) and its derivatives, including for example, homopolymers such as poly(aminocarboxylic acid), poly(acrylic acid), poly(methyl acrylic acid), copolymers of such homopolymers, and copolymers of poly(acrylic acid), acrylate, and/or acrylamide monomers such as those discussed above. Other pH sensitive polymers include polysaccharides such as cellulose acetate phthalate, hydroxypropylmethylcellulose phthalate, hydroxypropylmethylcellulose acetate succinate, cellulose acetate trimellilate, and chitosan. Yet other pH sensitive polymers include any mixture of a pH sensitive polymer and a water-soluble polymer.

Therapeutic agents can be delivered via polymeric carriers that are temperature sensitive (see e.g., Chen et al., Novel Hydrogels of a Temperature-Sensitive PLURONIC® Grafted to a Bioadhesive Polyacrylic Acid Backbone for Vaginal Drug Delivery, in PROCEED. INTERN. SYMP. CONTROL. REL. BIOACT. MATER. 22:167-168 (Controlled Release Society, Inc. 1995); Okano, Molecular Design of Stimuli-Responsive Hydrogels for Temporal Controlled Drug Delivery, in PROCEED. INTERN. SYMP. CONTROL. REL. BIOACT. MATER. 22:111-112 (Controlled Release Society, Inc. 1995); Johnston et al., PHARM. RES. 9(3):425-433 (1992); Tung, INT'L J. PHARM. 107:85-90 (1994); Harsh and Gehrke, J. CONTROLLED RELEASE 17:175-186 (1991); Bae et al., PHARM. RES. 8(4):531-537 (1991); Dinarvand and D'Emanuele, J. CONTROLLED RELEASE 36:221-227 (1995); Yu and Grainger, NOVEL THERMO-SENSITIVE AMPHIPHILIC GELS: POLY N—ISOPROPYLACRYLAMIDE-CO—SODIUM ACRYLATE-CO—N—N-ALKYLACRYLAMIDE NETWORK SYNTHESIS AND PHYSICOCHEMICAL CHARACTERIZATION 820-821 (Dept. of Chemical & Biological Sci., Oregon Graduate Institute of Science & Technology, Beaverton, Oreg.);, Zhou and Smid, PHYSICAL HYDROGELS OF ASSOCIATIVE STAR POLYMERS 822-823 (Polymer Research Institute, Dept. of Chemistry, College of Environmental Science and Forestry, State Univ. of New York, Syracuse, N.Y.); Hoffman et al., CHARACTERIZING PORE SIZES AND WATER ‘STRUCTURE’ 1N STIMULI-RESPONSIVE HYDROGELS 828 (Center for Bioengineering, Univ. of Washington, Seattle, Wash.); Yu and Grainger, THERMO-SENSITIVE SWELLING BEHAVIOR IN CROSSLINKED N—ISOPROPYLACRYLAMIDE NETWORKS: CATIONIC, ANIONIC AND AMPHOLYTIC HYDROGELS 829-830 (Dept. of Chemical & Biological Sci., Oregon Graduate Institute of Science & Technology, Beaverton, Oreg.); Kim et al., PHARM. RES. 9(3):283-290 (1992); Bae et al., PHARM. RES. 8(5):624-628 (1991); Kono et al., J. CONTROLLED RELEASE 30:69-75 (1994); Yoshida et al., J. CONTROLLED RELEASE 32:97-102 (1994); Okano et al., J. CONTROLLED RELEASE 36:125-133 (1995); Chun and Kim, J. CONTROLLED RELEASE 38:39-47 (1996); D'Emanuele and Dinarvand, INT'L J. PHARM. 118:237-242 (1995); Katono et al., J. CONTROLLED RELEASE 16:215-228 (1991); Hoffman, Thermally Reversible Hydrogels Containing Biologically Active Species, in POLYMERS IN MEDICINE III 161-167 (Migliaresi et al. eds., Elsevier Science Publishers B. V., Amsterdam, 1988); Hoffman, Applications of Thermally Reversible Polymers and Hydrogels in Therapeutics and Diagnostics, in THIRD INTERNATIONAL SYMPOSIUM ON RECENT ADVANCES IN DRUG DELIVERY SYSTEMS 297-305 (Salt Lake City, Utah, Feb. 24-27, 1987); Gutowska et al., J. CONTROLLED RELEASE 22:95-104 (1992); Palasis and Gehrke, J. CONTROLLED RELEASE 18:1-12 (1992); Paavola et al., PHARM. RES. 12(12): 1997-2002 (1995).

Representative examples of thermogelling polymers, and the gelatin temperature (LCST (° C.)) include homopolymers such as poly(N-methyl-N-n-propylacrylamide), 19.8; poly(N-n-propylacrylamide), 21.5; poly(N-methyl-N-isopropylacrylamide), 22.3; poly(N-n-propylmethacrylamide), 28.0; poly(N-isopropylacrylamide), 30.9; poly(N, n-diethylacrylamide), 32.0; poly(N-isopropylmethacrylamide), 44.0; poly(N-cyclopropylacrylamide), 45.5; poly(N-ethylmethyacrylamide), 50.0; poly(N-methyl-N-ethylacrylamide), 56.0; poly(N-cyclopropylmethacrylamide), 59.0; poly(N-ethylacrylamide), 72.0. Moreover thermogelling polymers may be made by preparing copolymers between (i.e., among) monomers of the above or by combining such homopolymers with other water-soluble polymers such as acrylmonomers (e.g., acrylic acid and derivatives thereof, such as methylacrylic acid, acrylate monomers, and derivatives thereof, such as butyl methacrylate, butyl acrylate, lauryl acrylate, and acrylamide monomers and derivatives thereof, such as N-butyl acrylamide and acrylamide).

Other representative examples of thermogelling polymers include cellulose ether derivatives such as hydroxypropyl cellulose, 41° C.; methyl cellulose, 55° C.; hydroxypropylmethyl cellulose, 66° C.; and ethylhydroxyethyl cellulose, polyalkylene oxide-polyester block copolymers of the structure X-Y, Y-X-Y and X-Y-X where X in a polyalkylene oxide and Y is a biodegradable polyester (e.g., PLC-PEG-PLC) and PLURONIC® polymers such as F-127, 10-15° C.; L-122, 19° C.; L-92, 26° C.; L-81, 200° C.; and L-61, 24° C.

Representative examples of patents relating to thermally gelling polymers and the preparation include U.S. Pat. Nos. 6,451,346; 6,201,072; 6,117,949; 6,004,573; 5,702,717; and 5,484,610, and PCT Pub. Nos. WO 99/07343, WO 99/18142, WO 03/17972, WO 01/82970, WO 00/18821, WO 97/15287, WO 01/41735, WO 00/00222, and WO 00/38651.

Therapeutic agents may be linked by occlusion in the polymer, dissolution in the polymer, bound by covalent linkages, bound by ionic interactions, or encapsulated in microcapsules. Within certain embodiments of the invention, therapeutic compositions are provided in non-capsular formulations such as microspheres (ranging from nanometers to micrometers in size), pastes, threads of various size, films, or sprays. In one aspect, the therapeutic agent may be incorporated into biodegradable magnetic nanospheres.

Within certain aspects of the present invention, therapeutic compositions may be fashioned in the form of microspheres, microparticles, and/or nanoparticles having any size ranging from 50 nm to 500 μm, depending upon the particular use. These compositions can be. These compositions can be formed by spray-drying methods, milling methods, coacervation methods, W/O emulsion methods, W/O/W emulsion methods, and solvent evaporation methods. In other aspects, these compositions can include microemulsions, emulsions, liposomes and micelles. Alternatively, such compositions may also be readily applied as a “spray,” which solidifies into a film or coating for use as a device/implant surface coating or to line the tissues of the implantation site. Such sprays may be prepared from microspheres of a wide array of sizes, including for example, from 0.1 μm to 3 μm, from 10 μm to 30 μm, and from 30 μm to 100 μm.

Therapeutic compositions may also be prepared in a variety of “paste” or gel forms. For example, within one embodiment of the invention, therapeutic compositions are provided which are liquid at one temperature (e.g., temperature greater than 37° C., such as 40° C., 45° C., 50° C., 55° C., or 60° C.), and solid or semi-solid at another temperature (e.g., ambient body temperature, or any temperature lower than 37° C.). Such “thermopastes” may be readily made utilizing a variety of techniques (see, e.g., PCT Pub. No. WO 98/24427). Other pastes may be applied as a liquid, which solidify in vivo due to dissolution of a water-soluble component of the paste and precipitation of encapsulated drug into the aqueous body environment. These “pastes” and “gels” containing therapeutic agents are particularly useful for application to the surface of tissues that will be in contact with an implant or device.

Within further aspects of the present invention, polymeric carriers are provided which are adapted to contain and release a hydrophobic compound, and/or the carrier containing the hydrophobic compound in combination with a carbohydrate, protein, or polypeptide. Within certain embodiments, the polymeric carrier contains or comprises regions, pockets, or granules of one or more hydrophobic compounds. For example, within one embodiment of the invention, hydrophobic compounds may be incorporated within a matrix that contains the hydrophobic therapeutic compound, followed by incorporation of the matrix within the polymeric carrier. A variety of matrices can be utilized in this regard, including for example, carbohydrates and polysaccharides such as starch, cellulose, dextran, methylcellulose, sodium alginate, heparin, chitosan and hyaluronic acid, proteins or polypeptides such as albumin, collagen, and gelatin. Within alternative embodiments, hydrophobic compounds may be contained within a hydrophobic core, and this core contained within a hydrophilic shell.

The therapeutic agent may be delivered as a solution. The therapeutic agent can be incorporated directly into the solution to provide a homogeneous solution or dispersion. In certain embodiments, the solution is an aqueous solution. The aqueous solution may further include buffer salts, as well as viscosity modifying agents (e.g., hyaluronic acid, alginates, carboxymethylcellulose (CMC), and the like). In another aspect of the invention, the solution can include a biocompatible solvent or liquid oligomers and/or polymers, such as ethanol, DMSO, glycerol, PEG-200, PEG-300 or NMP. These compositions may further comprise a polymer such a degradable polyester, where the polyester may comprise the residues of one or more of the monomers selected from lactide, lactic acid, glycolide, glycolic acid, ε-caprolactone, γ-caprolactone, hydroxyvaleric acid, hydroxybutyric acid, beta-butyrolactone, γ-butyrolactone, γ-valerolactone, γ-decanolactone, δ-decanolactone, trimethylene carbonate, 1,4-dioxane-2-one or 1,5-dioxepan-2one, or block copolymers of the form X-Y, Y-X-Y, R-(Y-X)n, R-(X-Y)n and X-Y-X (where X in a polyalkylene oxide (e.g., poly(ethylene glycol, poly(propylene glycol) and block copolymers of poly(ethylene oxide) and poly(propylene oxide) (e.g., PLURONIC® and PLURONIC® R series of polymers) and Y is a biodegradable polyester, where the polyester may comprise the residues of one or more of the monomers selected from lactide, lactic acid, glycolide, glycolic acid, ε-caprolactone, γ-caprolactone, hydroxyvaleric acid, hydroxybutyric acid, beta-butyrolactone, γ-butyrolactone, γ-valerolactone, γ-decanolactone, δ-decanolactone, trimethylene carbonate, 1,4-dioxane-2-one or 1,5-dioxepan-2one (e.g., PLG-PEG-PLG) and R is a multifunctional initiator).

Within another aspect of the invention, the therapeutic agent can further comprise a secondary carrier. The secondary carrier can be in the form of microspheres (e.g., PLGA, PLLA, PDLLA, PCL, gelatin, polydioxanone, poly(alkylcyanoacrylate)), nanospheres (PLGA, PLLA, PDLLA, PCL, gelatin, polydioxanone, poly(alkylcyanoacrylate)), liposomes, emulsions, microemulsions, micelles (SDS, block copolymers of the form X-Y, Y-X-Y, R-(Y-X)n, R-(X-Y)n, and X-Y-X (where X in a polyalkylene oxide (e.g., poly(ethylene glycol, poly(propylene glycol) and block copolymers of poly(ethylene oxide) and poly(propylene oxide) (e.g., PLURONIC® and PLURONIC® R series of polymers) and Y is a biodegradable polyester, where the polyester may comprise the residues of one or more of the monomers selected from lactide, lactic acid, glycolide, glycolic acid, ε-caprolactone, γ-caprolactone, hydroxyvaleric acid, hydroxybutyric acid, beta-butyrolactone, γ-butyrolactone, γ-valerolactone, γ-decanolactone, δ-decanolactone, trimethylene carbonate, 1,4-dioxane-2-one or 1,5-dioxepan-2one (e.g., PLG-PEG-PLG), and R is a multifunctional initiator), zeolites, or cyclodextrins.

Other carriers that may likewise be utilized to contain and deliver therapeutic agents described herein include: hydroxypropyl cyclodextrin (Cserhati and Hollo, INT. J. PHARM. 108:69-75 (1994)), liposomes (see, e.g., Sharma et al., CANCER RES. 53:5877-5881 (1993); Sharma and Straubinger, PHARM. RES. 11(60):889-896 (1994); PCT Pub. No. WO 93/18751; U.S. Pat. No. 5,242,073), liposome/gel (PCT Pub. No. WO 94/26254), nanocapsules (Bartoli et al., J. MICROENCAPSULATION 7(2):191-197 (1990)), micelles (Alkan-Onyuksel et al., PHARM. RES. 11(2):206-212 (1994)), implants (Jampel et al., INVEST. OPHTHALM. VIS. SCIENCE 34(11):3076-3083 (1993); Walter et al., CANCER RES. 54:22017-2212 (1994)), nanoparticles (Violante and Lanzafame PAACR), nanoparticles—modified (U.S. Pat. No. 5,145,684), nanoparticles (surface modified) (U.S. Pat. No. 5,399,363), micelle (surfactant) (U.S. Pat. No. 5,403,858), synthetic phospholipid compounds (U.S. Pat. No. 4,534,899), gas borne dispersion (U.S. Pat. No. 5,301,664), liquid emulsions, foam, spray, gel, lotion, cream, ointment, dispersed vesicles, particles or droplets, solid or liquid aerosols, microemulsions (U.S. Pat. No. 5,330,756), polymeric shell (nano and microcapsule) (U.S. Pat. No. 5,439,686), emulsion (Tarr et al., PHARM RES. 4: 62-165 (1987)), nanospheres (Hagan et al., PROC. INTERN. SYMP. CONTROL REL. BIOACT. MATER. 22 (1995); Kwon et al., PHARM RES. 12(2):192-195; Kwon et al., PHARM RES. 10(7):970-974; Yokoyama et al., J. CONTR. REL. 32:269-277 (1994); Gref et al., SCIENCE 263:1600-1603 (1994); Bazile et al., J. PHARM. SCI. 84:493-498 (1994)) and implants (U.S. Pat. No. 4,882,168).

Within another aspect of the present invention, polymeric carriers can be materials that are formed in situ. In one embodiment, the precursors can be monomers or macromers that contain unsaturated groups that can be polymerized and/or cross-linked. The monomers or macromers can then, for example, be injected into the treatment area or onto the surface of the treatment area and polymerized in situ using a radiation source (e.g., visible or UV light) or a free radical system (e.g., potassium persulfate and ascorbic acid or iron and hydrogen peroxide). The polymerization step can be performed immediately prior to, simultaneously to or post injection of the reagents into the treatment site. Representative examples of compositions that undergo free radical polymerization reactions are described in PCT Pub. Nos. WO 01/44307, WO 01/68720, WO 02/072166, WO 03/043552, WO 93/17669, and WO 00/64977,U.S. Pat. Nos. 5,900,245; 6,051,248; 6,083,524; 6,177,095; 6,201,065; 6,217,894; 6,639,014; 6,352,710; 6,410,645; 6,531,147; 5,567,435; 5,986,043; 6,602,975. and U.S. Patent App. Pub. Nos. 2002/012796A1, 2002/0127266A1, 2002/0151650A1, 2003/0104032A1, 2002/0091229A1, and 2003/0059906A1.

In certain aspects, it is desirable to use compositions that can be administered as liquids, but subsequently form hydrogels at the site of administration. Such in situ hydrogel forming compositions can be administered as liquids from a variety of different devices, and are more adaptable for administration to any site, since they are not preformed. Examples of in situ forming hydrogels include photoactivatable mixtures of water-soluble co-polyester prepolymers and polyethylene glycol to create hydrogel barriers. Block copolymers of polyalkylene oxide polymers (e.g., PLURONIC® compounds) and poloxamers have been designed that are soluble in cold water, but form insoluble hydrogels that adhere to tissues at body temperature (see e.g., Leach, et al., AM. J. OBSTET. GYNECOL. 162:1317-1319 (1990)).

Using the foregoing polymeric carriers, the present invention provides for the delivery of therapeutic agents via polymeric crosslinked matrices that may be used to assist in repair of cartilage tissue. Such polymeric materials may be prepared from, e.g., (a) synthetic materials, (b) naturally-occurring materials, or (c) mixtures of synthetic and naturally occurring materials. The matrix may be prepared from, e.g., (a) a one-component, i.e., self-reactive, compound, or (b) two or more compounds that are reactive with one another. Typically, these materials are fluid prior to delivery, and thus can be sprayed or otherwise extruded from a delivery device (e.g., a syringe) in order to deliver the composition. After delivery, the component materials react with each other, and/or with the body, to provide the desired affect. In some instances, materials that are reactive with one another must be kept separated prior to delivery to the patient, and are mixed together just prior to being delivered to the patient, in order that they maintain a fluid form prior to delivery. In a preferred aspect of the invention, the components of the matrix are delivered in a liquid state to the desired site in the body, whereupon in situ polymerization occurs.

### Delivery Systems

Suitable delivery systems for the homogeneous dry powder composition and the two buffer solutions may involve a multi-compartment spray device, where one or more compartments contains the powder and one or more compartments contain the buffer solutions needed to provide for the aqueous environment, so that the composition is exposed to the aqueous environment as it leaves the compartment. Many devices that are adapted for delivery of multi-component tissue sealants/hemostatic agents are well known in the art and can also be used in the practice of the present invention. Alternatively, the composition can be delivered using any type of controllable extrusion system, or it can be delivered manually in the form of a dry powder, and exposed to the aqueous environment at the site of administration. The homogeneous dry powder composition and the two buffer solutions may be conveniently formed under aseptic conditions by placing each of the three ingredients (dry powder, acidic buffer solution, and basic buffer solution) into separate syringe barrels. For example, the composition, first buffer solution, and second buffer solution can be housed separately in a multiple-compartment syringe system having a multiple barrels, a mixing head, and an exit orifice. The first buffer solution can be added to the barrel housing the composition to dissolve the composition and form a homogeneous solution, which is then extruded into the mixing head. The second buffer solution can be simultaneously extruded into the mixing head. Finally, the resulting composition can then be extruded through the orifice onto a surface.

For example, the syringe barrels holding the dry powder and the basic buffer may be part of a dual-syringe system, e.g., a double barrel syringe as described in U.S. Pat. No. 4,359,049 to Redl et al. In this embodiment, the acid buffer can be added to the syringe barrel that also holds the dry powder, so as to produce the homogeneous solution. In other words, the acid buffer may be added (e.g., injected) into the syringe barrel holding the dry powder to thereby produce a homogeneous solution of the first and second components. This homogeneous solution can then be extruded into a mixing head, while the basic buffer is simultaneously extruded into the mixing head. Within the mixing head, the homogeneous solution and the basic buffer are mixed together to thereby form a reactive mixture. Thereafter, the reactive mixture is extruded through an orifice and onto a surface (e.g., tissue), where a film is formed, which can function as a sealant or a barrier, or the like. The reactive mixture begins forming a three-dimensional matrix immediately upon being formed by the mixing of the homogeneous solution and the basic buffer in the mixing head. Accordingly, the reactive mixture is preferably extruded from the mixing head onto the tissue very quickly after it is formed so that the three-dimensional matrix forms on, and is able to adhere to, the tissue.

Other systems for combining two reactive liquids are well known in the art, and include the systems described in U.S. Pat. Nos. 6,454,786 to Holm et al.; 6,461,325 to Delmotte et al.; 5,585,007 to Antanavich et al.; 5,116,315 to Capozzi et al.; and 4,631,055 to Redl et al.

### Storage and Handling

Because crosslinkable components containing electrophilic groups react with water, the electrophilic component or components are generally stored and used in sterile, dry form to prevent hydrolysis. Processes for preparing synthetic hydrophilic polymers containing multiple electrophilic groups in sterile, dry form are set forth in commonly assigned U.S. Pat. No. 5,643,464 to Rhee et al. For example, the dry synthetic polymer may be compression molded into a thin sheet or membrane, which can then be sterilized using gamma or, preferably, e-beam irradiation. The resulting dry membrane or sheet can be cut to the desired size or chopped into smaller size particulates.

Components containing multiple nucleophilic groups are generally not water-reactive and can therefore be stored either dry or in aqueous solution. If stored as a dry, particulate, solid, the various components of the crosslinkable composition may be blended and stored in a single container. Admixture of all components with water, saline, or other aqueous media should not occur until immediately prior to use.

In an alternative embodiment, the crosslinking components can be mixed together in a single aqueous medium in which they are both unreactive, i.e., such as in a low pH buffer. Thereafter, they can be sprayed onto the targeted tissue site along with a high pH buffer, after which they will rapidly react and form a gel.

Suitable liquid media for storage of crosslinkable compositions include aqueous buffer solutions such as monobasic sodium phosphate/dibasic sodium phosphate, sodium carbonate/sodium bicarbonate, glutamate or acetate, at a concentration of 0.5 to 300 mM. In general, a sulfhydryl-reactive component such as PEG substituted with maleimido groups or succinimidyl esters is prepared in water or a dilute buffer, with a pH of between around 5 to 6. Buffers with pKs between about 8 and 10.5 for preparing a polysulfhydryl component such as sulfhydryl-PEG are useful to achieve fast gelation time of compositions containing mixtures of sulfhydryl-PEG and SG-PEG. These include carbonate, borate and AMPSO (3-[(1,1-dimethyl-2-hydroxyethyl)amino]2-hydroxy-propane-sulfonic acid). In contrast, using a combination of maleimidyl PEG and sulfhydryl-PEG, a pH of around 5 to 9 is preferred for the liquid medium used to prepare the sulfhydryl PEG.

### Other Components

In order to enhance adhesive strength, it may be generally desirable to add a “tensile strength enhancer” to the composition. Such tensile strength enhancers preferably comprise micron-size (preferably 5 to 40 microns in diameter and 20 to 5000 microns in length), high tensile strength fibers, usually with glass transition temperatures well above 37° C.

Suitable tensile strength enhancers for use in the present invention include collagen fibers, polyglycolide and polylactide fibers, as well as other organic tensile strength enhancers and inorganic tensile strength enhancers. Suitable tensile strength enhancers are those that have inherent high tensile strength and also can interact by covalent or non-covalent bonds with the polymerized gel network. The tensile strength enhancer should bond to the gel, either mechanically or covalently, in order to provide tensile support.

The compositions of the invention may also be used for localized delivery of various drugs and other biologically active agents in conjunction with the repair of damaged cartilage tissue. Biologically active agents may be delivered from the composition to a local tissue site in order to facilitate tissue healing and regeneration. Preferred biologically active agents useful for the method of the present invention include angiogenesis inhibitors (also referred to as anti-angiogenic factors) and paclitaxel, its analogues and derivatives.

1. Angiogenesis Inhibitors

In one embodiment of the present invention, the pharmacologically active agent for use with the compositions of the present invention is an angiogenesis inhibitor (e.g., 2-ME (NSC-659853), PI-88 (D-mannose, O-6-O-phosphono-alpha-D-mannopyranosyl-(1-3)-O-alpha-D-mannopyranosyl-(1-3)-O-alpha-D-mannopyranosyl-(1-3)-O-alpha-D-mannopyranosyl-(1-2)-hydrogen sulfate), thalidomide (1H-isoindole-1,3(2H)-dione, 2-(2,6-dioxo-3-piperidinyl)-), CDC-394, CC-5079, ENMD-0995 (S-3-amino-phthalidoglutarimide), AVE-8062A, vatalanib, SH-268, halofuginone hydrobromide, atiprimod dimaleate (2-azaspivo(4.5)decane-2-propanamine, N,N-diethyl-8,8-dipropyl, dimaleate), ATN-224, CHI R-258, combretastatin A-4 (phenol, 2-methoxy-5-(2-(3,4,5-trimethoxyphenyl)ethenyl)-, (Z)-), GCS-100LE, or an analogue or derivative thereof). Additional examples of angiogenesis inhibitors include AG-12,958 (Pfizer), ATN-161 (Attenuon LLC), neovastat, an angiogenesis inhibitor from Jerina AG (Germany), NM-3 (Mercian), VGA-1155 (Taisho), FCE-26644 (Pfizer), FCE-26950 (Pfizer), FPMA (Meiji Daries), FR-111142 (Fujisawa), GGTI-298, GM-1306 (Ligand), CPA-1734 (Novartis), NNC-47-0011 (Novo Nordisk), herbamycin (Nippon Kayaku), lenalidomide (Celegene), IP-10(NIH), ABT-828 (Abbott), KIN-841 (Tokushima University, Japan), SF-1126 (Semafore Pharmaceuticals), laminin technology (NIH), CHIR-258 (Chiron), NVP-AEW541 (Novartis), NVP-AEW541 (Novartis), Vt16907 (Alchemia), OXI-8007 (Oxigene), EG-3306 (Ark Therapeutics), Maspin (Arriva), ABT-567 (Abbott), PPI-2458 (Praecis Pharmaceuticals), CC-5079, CC-4089 (Celgene), HIF-1alpha inhibitors (Xenova), S-247 (Pfizer), AP-23573 (Ariad), AZD-9935 (Astra Zeneca), mebendazole (Introgen Therapeutics), MetAP-2 inhibitors (GlaxoSmithKline), AG-615 (Angiogene Pharmaceuticals), Tie-2 antagonists (Hybrigenics), NC-381, CYC-381, NC-169, NC-219, NC-383, NC-384, NC-407 (Lorus Therapeutics), ATN-224 (Attenuon), ON-01370 (Onconova), Vitronectin antagonists (Amgen), SDX-103 (Salmedix), Vitronectin antagonists (Shire), CHP (Riemser), TEK (Amgen), Anecortave acetate (Alcon), T46.2 (Matrix Therapeutics), HG-2 (Heptagen), TEM antagonists (Genzyme), Oxi-4500 (Oxigene), ATN-161 (Attenuon), WX-293 (Wilex), M-2025 (Metris Therapeutics), Alphastatin (BioActa), YH-16 (Yantai Rongchang), BIBF-1120 (Boehringer Ingelheim), BAY-57-9352 (Bayer), AS-1404 (Cancer Research Technology), SC-77964 (Pfizer), glycomimetics (BioTie Therapies), TIE-2 Inhibitors (Ontogen), DIMI, Octamer (Octamer), ABR-215050 (Active Biotech), ABT-518 (Abbott), KDR inhibitors (Abbott), BSF-466895 (Abbott), SCH-221153 (Schering-Plough), DAC:antiangiogenic (ConjuChem), TFPI (EntreMed), AZD-2171 (Astra-Zenaca), CDC-394 (Celgene), LY290293 (Eli Lilly), IDN-5390 (Indena), Kdr Kinase Inhibitors (Merck), CT-113020, CT-116433, CT-116563, CT-31890, CT-32228) (Cell Therapeutics), A-299620 (Abbott), TWEAK Inhibitor (Amgen), VEGF modulators (Johnson and Johnson), Tum-N53, tumstatin (Genzyme), Thios-1, Thios-2 (Thios Pharmaceuticals), MV-6401 (Miravant Medical Technologies), Spisulosine (PharmaMar), CEP-7055 (Cephalon), AUV-201 (Auvation), LM-609 (Eli Lilly), SKF-106615 (AnorMED), Oglufanide disodium (Cytran), BW-114 (Phaminox), Calreticulin (NIH), WX-678 (Wilex), SD-7784 (Pfizer), WX-UK1 (Wilex), SH-268 (Schering AG), 2-Me-PGA (Celgene), S-137 (Pfizer), ZD-6126 (Angiogene Pharmaceuticals), SG-292 (SignalGen), Benefin (Lane Labs), A6, A36 (Angstrom), SB-2723005 (GlaxoSmithKline), SC-7 (Cell Therapeutics), ZEN-014 (AEterna Zentaris), 2-methoxyestradiol (EntreMed), NK-130119 (Nippon Kayaku), CC-10004 (Celgene), AVE-8062A (Ajinomoto), Tacedinaline (Pfizer), Actinonin (Tokyo Metropolitan Institute of Medical Science), Lenalidomide (Celgene), VGA-1155, BTO-956 (SRI International), ER-68203-00 (Eisai), CT-6685 (UCB), JKC-362 (Phoenix Pharmaceuticals), DMI-3798 (DMI Biosciences, Angiomate (Ipsen), ZD-6474 (AstraZeneca), CEP-5214 (Cephalon), Canstatin (Genzyme), NM-3 (Mercian), Oridigm (MediQuest Therapeutics), Exherin (Adherex), BLS-0597 (Boston Life Sciences), PTC-299 (PTC Therapeutics), NPI-2358 (Nereus Pharmaceuticals), CGP-79787 (Novartis), AEE-788 (Novartis), CKD-732 (Chong Kun Dang), CP-564959 (OSI Pharmaceuticals), CM-101 (CarboMed), CT-2584, CT3501 (Cell Therapeutics), combretastatin and analogues and derivatives thereof (such as combretastatin A-1, A-2, A-3, A-4, A-5, A-6, B-1, B-2, B-3, B-4, D-1, D-2, and combretastatin A-4 phosphate (Oxigene)), Rebimastat (Bristol-Meyers Squibb), Dextrin 2-sulfate (ML Laboratories), Cilengitide (Merk KGaA), NSC-706704 (Phaminox), KRN-951 (Kirin Brewery), Ukrain, NSC-631570 (Nowicky Pharma), Tecogalan sodium (Daiichi Pharmaceutical), Tz-93 (Tsumura), TBC-1635 (Encysive Pharmaceuticals), TAN-1120 (Takeda), Semaxanib (Pfizer), BDI-7800 (Biophanmacopae), SD-186, SD-983 (Bristol-Meyers Squibb), SB-223245 (GlaxoSmithKline), SC-236 (Pfizer), RWJ-590973 (Johnson and Johnson), ILX-1850 (Genzyme), SC-68488, S-836 (Pfizer), CG-55069-11 (CuraGen), Ki-23057 (Kirin Brewery), CCX-700 (Chemoentryx), Pegaptanib octasodium (Giled Sciences), or an analogue or derivative thereof). In other embodiments, the angiogenesis inhibitor may be a recombinant anti-angiogenic compound such as ANGIOCOL™ (available from Biostratum Inc., Durham, N.C.).

Other examples of angiogenesis inhibitors for use in the present compositions include mycophenolic acid, clotrimazole, doxorubicin, camptothecin, halofuginone, epothilones, parthenolide, vinka alkaloids (vinblastine and vincristine), suramin, cephalomannine, paclitaxel, paclitaxel derivatives and analogues (e.g., 7-epipaclitaxel and 10-deacetylbaccatin).

2. Paclitaxel and Analogues and Derivatives Thereof

In another embodiment of the present invention, the pharmacologically active agent for use with the compositions of the present invention is paclitaxel, a compound that disrupts mitosis (M-phase) by binding to tubulin to form abnormal mitotic spindles or an analogue or derivative thereof. Briefly, paclitaxel is a highly derivatized diterpenoid (Wani et al., J. AM. CHEM. SOC. 93:2325 (1971)) obtained from the harvested and dried bark of Taxus brevifolia (Pacific Yew) and Taxomyces Andreanae and Endophytic Fungus of the Pacific Yew (Stierle et al., SCIENCE 60:214-216 (1993)). “Paclitaxel” (which should be understood herein to include formulations, prodrugs, analogues and derivatives such as, for example, TAXOL® (Bristol Myers Squibb, New York, N.Y.), TAXOTERE®) (Aventis Pharmaceuticals, France), docetaxel, 10-desacetyl analogues of paclitaxel and 3′N-desbenzoyl-3′N-t-butoxy carbonyl analogues of paclitaxel) may be readily prepared utilizing techniques known to those skilled in the art (see, e.g., Schiff et al., NATURE 277:665-667 (1979); Long and Fairchild, CANCER RESEARCH 54:4355-4361 (1994); Ringel and Horwitz, J. NAT'L CANCER INST. 83(4):288-291 (1991); Pazdur et al., CANCER TREAT. REV. 19(4):351-386 (1993); TETRAHEDRON LETTERS 35(52):9709-9712 (1994); J. Med. Chem. 35:4230-4237 (1992); J. MED. CHEM. 34:992-998 (1991); J. NATURAL PROD. 57(10): 1404-1410 (1994); J. NATURAL PROD. 57(11):1580-1583 (1994); J. AM. CHEM. SOC. 110:6558-6560 (1988); PCT Pub. Nos. WO 94/07882, WO 94/07881, WO 94/07880, WO 94/07876, WO 93/23555, WO 93/10076, WO94/00156, WO 93/24476; WO 94/20089; Eur. Pat. Pub. No. 590267; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184, or paclitaxel may be obtained from a variety of commercial sources, including for example, Sigma Chemical Co., St. Louis, Mo. (T7402-from Taxus brevifolia).

Representative examples of paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol (2′/or 7-O-ester derivatives), (2′/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-γ-aminobutyryltaxol formate, 2′-acetyl taxol, 7-acetyl taxol, 7-glycine carbamate taxol, 2′-OH-7-PEG (5000) carbamate taxol, 2′-benzoyl and 2′,7-dibenzoyl taxol derivatives, other prodrugs (2′-acetyltaxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl) taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, taxol analogues with modified phenylisoserine side chains, TAXOTERE®, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin); and other taxane analogues and derivatives, including 14-beta-hydroxy-10 deacetybaccatin III, debenzoyl-2-acyl paclitaxel derivatives, benzoate paclitaxel derivatives, phosphonooxy and carbonate paclitaxel derivatives, sulfonated 2′-acryloyltaxol; sulfonated 2′-O-acyl acid paclitaxel derivatives, 18-site-substituted paclitaxel derivatives, chlorinated paclitaxel analogues, C4 methoxy ether paclitaxel derivatives, sulfenamide taxane derivatives, brominated paclitaxel analogues, Girard taxane derivatives, nitrophenyl paclitaxel, 10-deacetylated substituted paclitaxel derivatives, 14-beta-hydroxy-10 deacetylbaccatin III taxane derivatives, C7 taxane derivatives, C10 taxane derivatives, 2-debenzoyl-2-acyl taxane derivatives, 2-debenzoyl and -2-acyl paclitaxel derivatives, taxane and baccatin III analogues bearing new C2 and C4 functional groups, n-acyl paclitaxel analogues, 10-deacetylbaccatin III and 7-protected-10-deacetylbaccatin III derivatives from 10-deacetyl taxol A, 10-deacetyl taxol B, and 10-deacetyl taxol, benzoate derivatives of taxol, 2-aroyl-4-acyl paclitaxel analogues, orthro-ester paclitaxel analogues, 2-aroyl-4-acyl paclitaxel analogues and 1-deoxy paclitaxel and 1-deoxy paclitaxel analogues.

In one aspect, the cell cycle inhibitor is a taxane having the formula (IV):

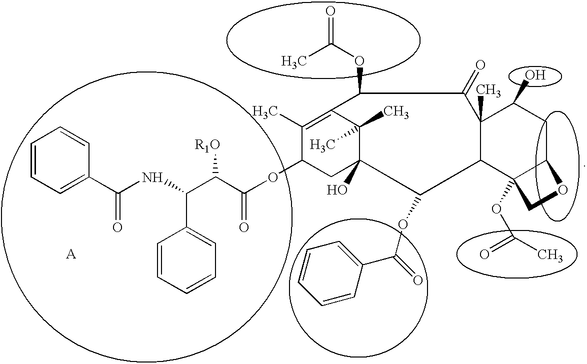

A side-chain (labeled “A” in the diagram) is desirably present in order for the compound to have good activity as a cell cycle inhibitor. Examples of compounds having this structure include paclitaxel (Merck Index entry 7117), docetaxol (TAXOTERE®, Merck Index entry 3458), and 3′-desphenyl-3′-(4-ntirophenyl)-N-debenzoyl-N-(t-butoxycarbonyl)-10-deacetyltaxol. Suitable taxanes such as paclitaxel and its analogues and derivatives are disclosed in U.S. Pat. No. 5,440,056 as having the structure (V):

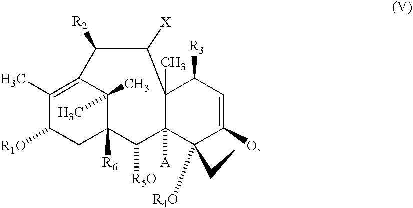

wherein X may be oxygen (paclitaxel), hydrogen (9-deoxy derivatives), thioacyl, or dihydroxyl precursors; R1 is selected from paclitaxel or TAXOTERE® side chains or alkanoyl of the formula (VI)

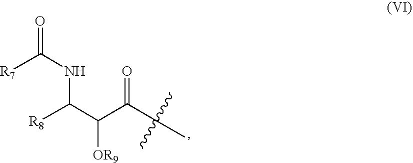

wherein R7 is selected from hydrogen, alkyl, phenyl, alkoxy, amino, phenoxy (substituted or unsubstituted); R8 is selected from hydrogen, alkyl, hydroxyalkyl, alkoxyalkyl, aminoalkyl, phenyl (substituted or unsubstituted), alpha or beta-naphthyl; and R9 is selected from hydrogen, alkanoyl, substituted alkanoyl, and aminoalkanoyl; where substitutions refer to hydroxyl, sulfhydryl, allalkoxyl, carboxyl, halogen, thioalkoxyl, N,N-dimethylamino, alkylamino, dialkylamino, nitro, and —OSO3H, and/or may refer to groups containing such substitutions; R2 is selected from hydrogen or oxygen-containing groups, such as hydrogen, hydroxyl, alkoyl, alkanoyloxy, aminoalkanoyloxy, and peptidyalkanoyloxy; R3 is selected from hydrogen or oxygen-containing groups, such as hydrogen, hydroxyl, alkoyl, alkanoyloxy, aminoalkanoyloxy, and peptidyalkanoyloxy, and may further be a silyl containing group or a sulphur containing group; R4 is selected from acyl, alkyl, alkanoyl, aminoalkanoyl, peptidylalkanoyl and aroyl; R5 is selected from acyl, alkyl, alkanoyl, aminoalkanoyl, peptidylalkanoyl and aroyl; R6 is selected from hydrogen or oxygen-containing groups, such as hydrogen, hydroxyl alkoyl, alkanoyloxy, aminoalkanoyloxy, and peptidyalkanoyloxy.

In another aspect, the paclitaxel analogues and derivatives useful as cell cycle inhibitors are disclosed in PCT Pub. No. WO 93/10076. As disclosed in this publication, the analogue or derivative should have a side chain attached to the taxane nucleus at C13, as shown in the structure below (formula VII), in order to confer antitumor activity to the taxane.

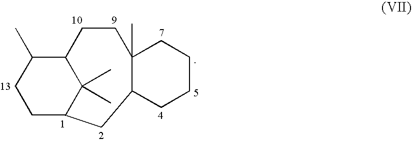

PCT Pub. No. WO 93/10076 discloses that the taxane nucleus may be substituted at any position with the exception of the existing methyl groups. The substitutions may include, for example, hydrogen, alkanoyloxy, alkenoyloxy, aryloyloxy. In addition, oxo groups may be attached to carbons labeled 2, 4, 9, and/or 10. As well, an oxetane ring may be attached at carbons 4 and 5. As well, an oxirane ring may be attached to the carbon labeled 4.

In a further aspect, a taxane-based cell cycle inhibitor useful for use with the compositions of the present invention is disclosed in U.S. Pat. No. 5,440,056, which discloses 9-deoxo taxanes. These are compounds lacking an oxo group at the carbon labeled 9 in the taxane structure shown above (formula VIII). The taxane ring may be substituted at the carbons labeled 1, 7, and 10 (independently) with H, OH, O—R, or O—CO—R where R is an alkyl or an aminoalkyl. As well, it may be substituted at carbons labeled 2 and 4 (independently) with aryol, alkanoyl, aminoalkanoyl or alkyl groups. The side chain of formula (VI) may be substituted at R7 and R8 (independently) with phenyl rings, substituted phenyl rings, linear alkanes/alkenes, and groups containing H, O or N. R9 may be substituted with H, or a substituted or unsubstituted alkanoyl group.

Taxanes in general and paclitaxel in particular, are considered to function as cell cycle inhibitors by acting as an anti-microtubule agent, and more specifically as stabilizers. These compounds have been shown useful in the treatment of proliferative disorders, including: non-small cell (NSC) lung, small cell lung, breast, prostate, cervical, endometrial, head, and neck cancers.

3. Dosages

Drug doses administered from the compositions of the present invention for the repair of cartilage depends on a variety of factors, including the type of formulation and treatment site; however, certain principles can be applied in the application of this art. Drug dose can be calculated as a function of dose per unit area (of the treatment site); using this calculation, thetotal drug dose administered can be measured and appropriate surface concentrations of active drug can be determined. For local application (such as intra-articular or endoscopic administration), drugs are to be used at concentrations that range from several times more than to 50%, 20%, 10%, 5%, or even less than 1% of the concentration typically used in a single systemic dose application. In certain aspects, the agent is released from the composition in effective concentrations in a time period that may be measured from the time of infiltration into tissue, which ranges from about less than 1 day to about 180 days. Release time may also be from about 7 days to about 14 days; from about 14 days to about 28 days; from about 28 days to about 56 days; from about 56 days to about 90 days; or from about 90 days to about 180 days. In one aspect, the drug is released in effective concentrations for a period ranging from 1-90 days.

Therapeutic agents, used alone or in combination, may be administered under the following dosing guidelines. The total amount (dose) of therapeutic agent in the composition can be in the range of about 0.01 μg-10 μg; 10 μg-10 mg; 10 mg-250 mg; 250 mg-1000 mg; or 1000 mg-2500 mg. The dose (i.e., amount) of therapeutic agent per unit area of surface to which the agent is applied may be in the range of about 0.01 μg/mm²-1 μg/mm²; 1 μg/mm²-10 μg/mm²; 10 μg/mm²-250 μg/mm²; 250 μg/mm²-1000 μg/mm²; or 1000 μg/mm²-2500 μg/mm².

Provided below are exemplary dosage ranges for angiogenesis inhibitors and paclitaxel and analogues and derivatives thereof, i.e., preferred therapeutic agents that can be used in conjunction with compositions of the present invention for the repair of cartilage. (A) Angiogenesis inhibitors include without limitation, alphastatin, ZD-6474, IDN-5390, SB-2723005, ABT-518, combretastatin, anecortane, and analogues and derivatives thereof. The total dose of angiogenesis inhibitors in the composition is typically not to exceed 200 mg (range of 0.1 μg to 200 mg), with a preferred total dose in the range of 1 μg to 100 mg. The dose per unit area (e.g., when used on a tissue or implant surface) of angiogenesis inhibitors is typically in the range of 0.01 μg-100 μg per mm², with a preferred dose per unit area in the range of 0.1 μg/mm²-20 μg/mm². (B) Paclitaxel and analogues and derivatives (e.g., docetaxel) typically have a total dose in the composition not to exceed 10 mg (range of 0.1 μg to 10 mg), with a preferred total dose in the range of 1 μg to 3 mg. The dose per unit area (e.g., when used on a tissue or implant surface) of paclitaxel, its analogues and derivatives, is typically in the range of 0.1 μg-10 μg per mm², with a preferred dose per unit area in the range of 0.25 μg/mm²-5 μg/mm².

Other active agents for use in the compositions of the present invention are small molecules including but not limited to dexamethasone, isotretinoin (13-cis retinoic acid), 17-β-estradiol, estradiol, 1-α-25 dihydroxyvitamin D₃ diethylstibesterol, cyclosporin A, L-NAME (a non-selective inhibitor of nitric oxide synthase), all-trans retinoic acid (ATRA), and analogues and derivatives thereof, cytokines, such as TNFα, NGF, GM-CSF, IL-1, IL-1-βP, IL-8, IL-6, growth hormone, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogues, fragments, and derivatives of such growth factors. Members of the TGF supergene family include the beta transforming growth factors (e.g., TGF-β₁, TGF-β₂, TGF-β₃); bone morphogenetic proteins (e.g., BMP-1, BMP-2, BMP-3, BMP-4, BMP-5, BMP-6, BMP-7, BMP-8, BMP-9); heparin-binding growth factors (e.g., fibroblast growth factor (FGF), epidermal growth factor (EGF), platelet-derived growth factor (PDGF), insulin-like growth factor (IGF)); inhibins (e.g., inhibin A, inhibin B); growth differentiating factors (e.g., GDF-1); and activins (e.g., activin A, activin B, activin AB). Growth factors can be isolated from native or natural sources, such as from mammalian cells, or can be prepared synthetically, such as by recombinant DNA techniques or by various chemical processes. In addition, analogues, fragments, or derivatives of these factors can be used, provided that they exhibit at least some of the biological activity of the native molecule. For example, analogues can be prepared by expression of genes altered by site-specific mutagenesis or other genetic engineering techniques.

Bone morphogenic proteins (e.g., BMP-2, BMP-3, BMP-4, BMP-5, BMP-6, or BMP-7 or an analogue or derivative thereof) are to be used in formulations at concentrations that range from 0.0001 μg/mL to approximately 25 mg/mL depending on the specific clinical application, formulation type (e.g., gel, liquid, solid, semi-solid), formulation chemistry, duration of required application, type of medical device interface and formulation volume and or surface area coverage required. Preferably, the bone morphogenic protein is released in effective concentrations for a period ranging from 1-180 days. The total dose for a single application is typically not to exceed 30 mg (range of 0.001 μg to 30 mg); preferred 1 μg to 20 mg and the dose per unit area of tissue is in the range of 0.001 μg-1000 μg per mm²; with a preferred dose of 0.01 μg/mm²-200 μg/mm². Minimum concentration of 10⁻⁹-10⁻⁴ M of bone morphogenic protein is to be maintained on the device surface.

Cytokines useful in the method of the present invention include without limitation, cytokines such as TNFα, NGF, GM-CSF, IL-1, IL-1-β, IL-8, IL-6, growth hormone, transforming growth factors (TGFs), fibroblast growth factors (FGFs), platelet derived growth factors (PDGFs), epidermal growth factors (EGFs), connective tissue activated peptides (CTAPs), osteogenic factors, and biologically active analogues, fragments, and derivatives of such growth factors, members of the TGF supergene family including the beta transforming growth factors (e.g., TGF-β₁, TGF-β₂, TGF-β₃). Cytokines are preferably used in the compositions of the present invention at concentrations that range from 0.0001 μg/mL to approximately 20 mg/mL depending on the specific clinical application, formulation type (e.g., gel, liquid, solid, semi-solid), formulation chemistry, duration of required application, and formulation volume and/or surface area coverage required. Preferably, the cytokine and/or growth factor is released in effective concentrations for a period ranging from 1-180 days. The total dose for a single application is typically not to exceed 100 mg (range of 0.0001 μg to 100 mg); preferred 0.001 μg to 30 mg. When used on a tissue or implant surface, the dose per unit area is in the range of 0.0001 μg-500 μg per mm²; with a preferred dose of 0.001 μg/mm²-200 μg/mm². The minimum concentration of inflammatory cytokine to be maintained on a tissue or implant surface is in the range of 10⁻¹⁰-10⁻⁴ g/mL.

Furthermore, the composition may alone or additionally comprise an agent that stimulates processes involving tissue regeneration including but not limited to cellular proliferation, cell migration, and/or cell adhesion. Examples include: dexamethasone, RGD (Arg-Gly-Asp) sequence, isotretinoin (13-cis retinoic acid), 17-β-estradiol, estradiol, 1-α-25 dihydroxyvitamin D₃, diethylstibesterol, cyclosporin A, L-NAME (a non-selective inhibitor of nitric oxide synthase), all-trans retinoic acid (ATRA), and analogues and derivatives thereof. Doses used are those concentrations which are demonstrated to stimulate cell proliferation (see, Examples 8-13), migration (see, Example 14) and/or cell adhesion and/or other processes involved in tissue regeneration. The agents are to be used in formulations at concentrations that range from 0.1 μg to 25 mg/mL depending on the specific clinical application, formulation type (e.g., gel, liquid, solid, semi-solid), formulation chemistry, duration of required application, type of medical device interface and formulation volume and or surface area coverage required. Preferably, the agent is released in effective concentrations for a period ranging from 1-180 days. The total dose for a single application is typically not to exceed 200 mg (range of 0.0001 μg to 200 mg); preferred 0.001 μg to 100 mg. When used on a tissue or implant surface, the dose is per unit area is in the range of 0.00001 μg-500 μg per mm²; with a preferred dose in the range of 0.0001 μg/mm²-200 μg/mm². The minimum concentration of proliferative agent to be maintained on a tissue or implant surface is in the range of 10⁻¹¹-10⁻⁶ M.

Biologically active agents may be incorporated into the composition by admixture. Alternatively, the agents may be incorporated into the crosslinked polymer matrix by binding these agents to the functional groups on the synthetic polymers. Processes for covalently binding biologically active agents such as growth factors using functionally activated polyethylene glycols are described in commonly owned U.S. Pat. No. 5,162,430 to Rhee et al. Such compositions preferably include linkages that can be easily biodegraded, for example as a result of enzymatic degradation, resulting in the release of the active agent into the target tissue, where it will exert its desired therapeutic effect.

A simple method for incorporating biologically active agents containing nucleophilic groups into the crosslinked polymer composition involves mixing the active agent with a polyelectrophilic component prior to addition of the polynucleophilic component.

By varying the relative molar amounts of the different components of the crosslinkable composition, it is possible to alter the net charge of the resulting crosslinked polymer composition, in order to prepare a matrix for the delivery of a charged compound such as a protein or ionizable drug. As such, the delivery of charged proteins or drugs, which would normally diffuse rapidly out of a neutral carrier matrix, can be controlled.

For example, if a molar excess of a polynucleophilic component is used, the resulting matrix has a net positive charge and can be used to ionically bind and deliver negatively charged compounds. Examples of negatively charged compounds that can be delivered from these matrices include various drugs, cells, proteins, and polysaccharides. Negatively charged collagens, such as succinylated collagen, and glycosaminoglycan derivatives such as sodium hyaluronate, keratan sulfate, keratosulfate, sodium chondroitin sulfate A, sodium dermatan sulfate B, sodium chondroitin sulfate C, heparin, esterified chondroitin sulfate C, and esterified heparin, can be effectively incorporated into the crosslinked polymer matrix as described above.

If a molar excess of a polyelectrophilic component is used, the resulting matrix has a net negative charge and can be used to ionically bind and deliver positively charged compounds. Examples of positively charged compounds that can be delivered from these matrices include various drugs, cells, proteins, and polysaccharides. Positively charged collagens, such as methylated collagen, and glycosaminoglycan derivatives such as esterified deacetylated hyaluronic acid, esterified deacetylated desulfated chondroitin sulfate A, esterified deacetylated desulfated chondroitin sulfate C, deacetylated desulfated keratan sulfate, deacetylated desulfated keratosulfate, esterified desulfated heparin, and chitosan, can be effectively incorporated into the crosslinked polymer matrix as described above.

Varying the relative molar amounts of the different components of the crosslinkable composition may be used to alter the crosslinked compositions mechanical properties.

The compositions can also be prepared to contain various colorants or imaging agents such as synthetic dyes and natural coloring agents, light emissive and fluorescent dyes, iodine or barium sulfate, or fluorine, in order to aid visualization of the compositions after administration via optical, X-ray or ¹⁹F-MRI detection means. Suitable colorants include, but are not limited to, FD&C dyes and FD&C lakes, (e.g., allura red AC, amaranth, brilliant blue FCF, quinoline yellow, sunset yellow FCF), black PN, Bordeaux B, Brown FK, Brown HT, canthaxanthin, carmine, carmoisine, beetroot red, chlorophyll, conchineal, curcumin, eosin, erythrosine, green S, ponceau 4R, red 2G, saffron, tartrazine, turmeric, and mixtures thereof. Examples of light-emissive and fluorescent dyes include: fluorescein, rose bengal, indocyanine green, analogue members of the tricarbocyanine dyes; and many others. In selecting a suitable dye, color and luminescent efficiency are two important factors. Luminescent dyes found particularly suitable include cyanine and related polymethine dyes, merocyanine, styryl and oxonol dyes. Other suitable coloring agents, light-emissive dyes, and fluorescent dyes will be obvious to those skilled in the art. It may also be desirable to incorporate proteins such as albumin, fibrin, or fibrinogen into the crosslinked polymer composition to promote cellular adhesion. In addition, the introduction of hydrocolloids such as carboxymethylcellulose may promote tissue adhesion.

### Crosslinking of the Composition

Any number of crosslinking techniques may be used to effect crosslinking of the aforementioned compositions. Generally, however, components A, B and optionally C are selected such that crosslinking occurs fairly rapidly upon admixture of all components of the crosslinkable composition with an aqueous medium.

For crosslinking compositions in which one or more components contain hydroxyl and/or thiol groups as nucleophilic moieties, the aqueous medium with which the crosslinking composition (or components thereof) are admixed should contain a basic reagent that is effective to increase the nucleophilic reactivity of the hydroxyl and/or thiol group (and thus the rate of the nucleophile-electrophile reactions) but that is preferably non-nucleophilic so as to avoid reaction with any electrophilic groups present. A catalytic amount of base can be used, and/or a base-containing buffer. In an alternative embodiment, a reactive base can be used that participates as a reactant in the crosslinking reaction.

In general, the combined concentration of all crosslinkable components in the aqueous admixture will be in the range of about 1 to 50 wt. %, generally about 2 to 40 wt. %; however, a preferred concentration of the crosslinkable composition in the aqueous medium, as well as the preferred concentration of each crosslinkable component therein, will depend on a number of factors including the type of component, its molecular weight, and the end use of the composition. For example, use of higher concentrations of the crosslinkable components, or using highly functionalized components, will result in the formation of a more tightly crosslinked network, producing a stiffer, more robust gel. As such, compositions intended for use in tissue augmentation will generally employ concentrations of crosslinkable components that fall toward the higher end of the preferred concentration range. The appropriate concentration of each crosslinkable component can easily be optimized to achieve a desired gelation time and gel strength using routine experimentation.

Using the composition disclosed above, elastic modulus values ranging from approximately 2 N/cm² to approximately 40 N/cm² have been observed during in vitro testing. Average tensile strengths ranging from 1.5 N/cm² to 70 N/cm² have been observed in tests using an INSTRON® testing apparatus.

### Kits

In one aspect of the invention, there is provided a kit for repairing damaged cartilage or other connective tissue, wherein the kit comprises: (a) a first hydrophilic polymer; (b) a crosslinkable component A having m nucleophilic groups, wherein m≧2; and (c) a crosslinkable component B having n electrophilic groups capable of reaction with the m nucleophilic groups to form covalent bonds, wherein n≧2 and m+n≧4, wherein each of components A and B is biocompatible and nonimmunogenic, and at least one of components A and B is a second hydrophilic polymer, and reaction of the components results in a biocompatible, nonimmunogenic, crosslinked matrix.

In a preferred embodiment of the kit, each of components A and B comprise polyalkeleneoxide. In a more preferred embodiment, the polyalkyleneoxide is a poly(ethylene oxide). Within the kit, it is contemplated that components A and B may be the same or different. Components A and B may also be in admixture in a liquid or a solid form.

In yet another aspect of the invention, the kit further comprises a device for mixing (a), (b), and (c) and delivering (a), (b), and (c) or a partial reaction product thereof to the damaged cartilage or other connective tissue. The device may be configured to spray material onto a surface of the damaged cartilage tissue, muscle, periosteum, ligament, tendon fat, and/or soft tissue implant. For example, the device may be configured to deliver material onto a surface of the damaged cartilage or other connective tissue as a liquid, gel, or suspension.

In a preferred embodiment of the kit, the first hydrophilic polymer is methylated collagen dissolved or suspended in aqueous solution of pH less than 7.

In still a further aspect of the invention, the kit further comprises an additional component (d), which comprises an aqueous solution of pH greater than 7.

In yet another aspect of the invention, there is provided a kit for repairing damaged cartilage or other connective tissue, wherein the kit comprises: (a) an aqueous solution of methylated collagen, the solution having a pH of less than 7; (b) (pentaerythritol tetrakis[mercaptoethyl poly(oxyethylene) ether]; and (c) pentaerythritol tetrakis [1-(1′-oxo-5-succimidylpentanoate)-2-poly(oxyethylene) ether], wherein (b) and (c) are in admixture in solid form; and (d) an aqueous solution having a pH of greater than 7.

In one embodiment of the kit, there is included one PEG pouch, one collagen pouch, and one buffer pouch. Within this kit, the PEG pouch is comprised of PEG in a 50:50 mixture of two synthetic polymers, both approximately 10,000 MW: pentaerythritol poly(ethylene glycol) either tetra succinimidyl glutarate (COH102) and pentaerythritol poly(ethylene glycol) ether tetra-thiol (COH206); the collagen pouch is contains one syringe filled with 20-22 mg/mL methylated bovine collagen in sodium acetate/sodium chloride solution at pH 3.5 to 4.0; and the buffer pouch contains one syringe filled with sodium phosphate and sodium carbonate, pH 9.6 comprised of 120 mM sodium phosphate and 180 mM sodium bicarbonate.

It is to be understood that while the invention has been described in conjunction with the preferred specific embodiments thereof the foregoing description is intended to illustrate and not limit the scope of the invention. Other aspects, advantages, and modifications will be apparent to those skilled in the art to which the invention pertains. All patents, patent applications, patent publications, journal articles and other references cited herein are incorporated by reference in their entireties.

### EXPERIMENTAL

The following examples are put forth so as to provide those of ordinary skill in the art with a complete disclosure and description of how to make and use the compounds of the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric.

From the foregoing description of the embodiments of the invention and from the following examples, it will be appreciated that although specific embodiments and examples of the invention have been described herein for purposes of illustration, various modifications may be made without deviating from the spirit and scope of the invention; accordingly, the invention is not limited by the description for the embodiments or the examples as set forth herein.

In the following examples, the term “NHS-PEG” refers to pentaerythritol tetrakis[1-(1′-oxo-5-succimidylpentanoate)-2-poly(oxyethylene)]ether, also known as tetra functional succinimidyl glutarate PEG (i.e., a four-armed NHS-PEG) with a molecular weight of 10,000. The term “HS-PEG” refers to pentaerythritol tetrakis[mercaptoethyl poly(oxyethylene)]ether, also known as tetra functional thiol PEG (i.e., a four-armed thiol PEG) with a molecular weight of 10,000. Both NHS-PEG and HS-PEG are available from Aldrich Chemical Co., Milwaukee, Wis.

### EXAMPLE 1

### Gelation OF NHS-PEG and HS-PEG

A homogenous mixture of NHS-PEG and HS-PEG was obtained by mixing approximately equal amounts of the two powders. A 20% (w/v) solution of mixed PEG powder was then prepared by dissolving the powder in diluted HCl. The obtained solution (pH 2.1) was then cosprayed with an equal volume of a 300 mM sodium phosphate/sodium carbonate buffer (pH 9.6). Gelation occurred almost immediately (<3 sec) and the gel obtained its firm, rubbery, solid properties in less than a minute with a total solid content of approximately 10% PEG.

### EXAMPLE 2

### Gelation of NHS-PEG, HS-PEG, and Methylated Collagen

A crosslinked hydrogel was formed from an acidic solution of NHS-PEG, HS-PEG, and a methylated collagen (MC). The acidic paste (pH 3-4) containing 10% NHS-PEG, 10% HS-PEG, and 22 mg/mL of MC spontaneously gelled when mixed with an equal volume of 0.3 M phosphate/carbonate solution (pH 9.6). Gelation occurred within seconds to form a strong hydrogel that adheres well to the tissue and swells in a controlled fashion in saline solution. As previously noted, the combination of NHS-PEG (10K), HS-PEG (10K), and MC is associated with the tradename CHONDROGEL™.

### EXAMPLE 3

### Gelation of NHS-PEG, HS-PEG, and MC on a Tissue Sample

A crosslinked hydrogel (i.e., CHONDROGEL™) was formed from an acidic solution of NHS-PEG, HS-PEG, and a methylated collagen (MC). The acidic paste (pH 3-4) containing 10% NHS-PEG, 10% HS-PEG, and 22 mg/mL of MC was applied onto a tissue and shaped with a spatula to its desired form. In a subsequent step, a smaller volume of 0.3 M phosphate/carbonate solution (pH 9.6) than that used in Example 2 was applied (via spraying or dropping) to the paste. Gelation of the paste started immediately and continued as the 0.3 M phosphate/carbonate solution diffused into the tissue. Within a few minutes, a strong hydrogel that adhered well to the tissue and swells in a controlled fashion in saline solution was formed on the tissue.

### EXAMPLE 4

### Gelation OF NHS-PEG and MC

10% w/v of NHS-PEG was mixed with MC at a concentration of 19 mg/ml and pH 3-4 to form a homogenous stable acidic solution. This solution spontaneously formed a three-dimensional matrix when mixed with 0.3 M phosphate/carbonate pH 9.6 solution. The basic solution activated the amines from the MC, which then reacted with the SG (succinimidyl glutarate) groups to form the amide bonds. The gelation occurred within seconds to form a strong biomaterial that adhered well to the tissue and did not swell after 24 hours of immersion in saline solution.

### EXAMPLE 5

### Use of the Composition for the Repair of Lateral Meniscus

To repair the lateral meniscus in a patient, the patient is placed in a supine position with the knee of the affected leg positioned at a 90-degree angle to facilitate access to the lateral meniscus during the fixation and/or suturing procedures. The supine position is the most convenient position to make a posterolateral skin incision if inside-out suturing is done.

For the surgical exposure, standard anteromedial and anterolateral portals are made. The following landmarks are used to identify the correct position of the arthroscopic portals: the anterolateral portal is placed 1 cm superior to the distal pole patella and a thumb's breadth lateral to the patella; the anteromedial portal is placed at the level of the distal pole of the patella and a thumb's breadth medial to the patella.

During the surgical procedure the surgeon may require accessory portals in order to obtain the desired view or access. Portals slightly higher and more medial and lateral to the “normal” position have been shown to be useful. If anatomy dictates, such as with a prominent tibial spine, a midline transpatellar tendon portal might be useful to get optimal access to the most posterior aspect of the meniscus during suturing.

Several options are available for repairing a meniscus. A fairly standard and well-established technique involves the inside-out suture technique using the SHARPSHOOTER® Tissue Repair System (ReGen Biologics, Franklin Lakes, N.J.) or a hand-held zone specific cannulae. An alternative method, which to date has limited clinical experience, involves an all-inside suture fixation technique. Both techniques are described below in detail.

The Inside-Out Technique:

To conduct the inside-out technique for the repair of a lateral meniscus, a posterolateral skin incision is necessary in order to assist in the identification and capture of the needles during the lateral inside-out suturing procedure and to ensure the proper identification and protection of neurovascular structures. The incision should be approximately 3-5 cm in length, ⅓ above, and ⅔ below the lateral joint line and in the interval between the posterior edge of the iliotibial band and the anterior edge of the biceps tendon. The incision is deepened with blunt dissection until the capsule is reached. A tissue protection spoon retractor is placed in the incision as deeply as possible. This instrument will help to protect the neurovascular structures while the suture needles are passed from the inside of the joint to the lateral outer aspects of the knee.

Next, CHONDROGEL™ is injected into the meniscal defect at an amount approximated to fill the defect, preferably with an 18G needle; any excess CHONDROGEL™ will be displaced out of the meniscus. As previously noted, CHONDROGEL™, available from Angiotech Biomaterials Corp. (Palo Alto, Calif.) is the tradename for a combination of a 4-armed thiol PEG (10K), a 4-armed NHS PEG (10K), and methylated collagen. By using the SHARPSHOOTER® Tissue Repair System, or conventional inside-out suturing cannulae, the meniscus is sutured together with size 2-0 braided polyester. The suturing can be done either from the posterior to the anterior end of the implant or vice versa. A change of suturing direction during the suturing procedure is not recommended. The first horizontal suture is placed either in the anterior or posterior aspect of the meniscus defect. Using a double-armed suture, the first arm is typically placed into the intact meniscus remnant and the second arm into the torn meniscus. Vertical mattress sutures are used throughout the entire length of the implant, other than the ends where mattress sutures are used. Sutures should be placed every 4-5 mm.

When using the SHARPSHOOTER® Tissue Repair System, it is helpful to advance the needle of the second arm about 2 mm out of the cannula before placing the stitch. With the advanced needle, the torn meniscus can be picked up approximately mid-width. With the application of slight posterior pressure, the proper positioning of the implant is assured before advancing the needle. Care must be taken to ensure that the two arms of the sutures are pulled together at the same time to avoid any sawing action of the suture on the torn fragment.

After placement of all sutures, the sutures should be tied over the capsule through the posterolateral skin incision while the torn fragment remains under visual control of the arthroscope. When tying the sutures, the tension should allow apposition of torn meniscus to the meniscus rim. Direct visualization can help to assure that the sutures are tied with correct tension.

The All-Inside Fixation Technique:

The all-inside fixation technique does not require additional skin incisions over those required for the inside-out suturing described above.

A standard curve FAST-FIX® from Smith & Nephew (London, GB) is used in this procedure in accordance with the manufacturer's instructions. The white plastic sheath that acts as a depth guard typically should be cut to 16 mm before insertion into the joint. This length should be adequate to allow the tip of the device to penetrate the torn mensicus and the meniscus rim while preventing the tip from jeopardizing the neurovascular structures.

With the torn fragment positioned properly in the meniscus defect, the torn fragment can be attached to the meniscus rim similar to that noted above for the inside-out suture technique. Horizontal pattern stitches should be placed in the anterior and posterior end, and vertical pattern stitches should be placed over the remainder of the torn fragment. The FAST-FIX® first penetrates the torn fragment with the curve pointed posteriorly toward the neurovascular structures. The device is then rotated 180 degrees so the curve points anteriorly away from the neurovascular structures as it is advanced and penetrates the meniscus rim. When the tip can be felt to penetrate the meniscus rim, or when the depth guard prevents further penetration, the thumb slide is advanced so that the first fixation implant, T1, is deployed from the needle. The FAST-FIX® needle is then backed out of the tissue and into the joint carefully to assure that T1 is secure against the peripheral meniscus rim. While the tip of the FAST-FIX® can be visualized, the thumb slide is advanced to position the second implant, T2, at the tip of the needle. Care must be taken not to allow the sutures to become tangled during this maneuver. The FAST-FIX® needle is then passed into the host meniscus rim, either vertically or horizontally as noted above. When the tip can be felt to penetrate the meniscus rim, the thumb slide is advanced so that T2 is deployed from the needle. The FAST-FIX® device is then completely removed from the joint with care to assure that the suture end also is exteriorized. Under direct arthroscopic visualization, the suture is tightened down either by hand or with the assistance of a knot pusher. Care should be taken not to over tighten the suture otherwise the torn fragment could be cut by a too tight suture. Using the suture cutter or arthroscopic scissors, the suture is cut just proximal to the knot. This procedure is repeated to place as many fixation devices as necessary to obtain a complete and secure fixation.

### EXAMPLE 6

### Use of the Composition for the Repair of Medial Mensicus

Preoperative surgical planning for the repair of a medial mensicus is based on clinical, radiographic and MRI examinations. For the repair a medial meniscus, the patient is placed in a supine position with the knee of the affected leg positioned at a 90 degree angle to facilitate access to the medial meniscus during the later-stage suturing procedure and also to facilitate retrieval of the needles from the posteromedial skin incision.

For the surgical exposure, standard anteromedial and anterolateral portals are made. The following landmarks are used to identify the correct position of the arthroscopic portals: the anterolateral portal is placed 1 cm superior to the distal pole patella and a thumb's breadth lateral to the patella; the anteromedial portal is placed at the level of the distal pole of the patella and a thumb's breadth medial to the patella.

During the surgical procedure the surgeon may require accessory portals in order to obtain the desired view or access. Portals slightly higher and more medial and lateral to the “normal” position have been shown to be useful. If anatomy dictates, such as a prominent tibial spine, a midline transpatellar tendon portal might be useful to get optimal access to the most posterior aspect of the meniscus during suturing.

For the preparation of the implant bed, the site should be roughened by using an awl or rasping instrument to which the CHONDROGEL™ may be applied through the scope portal or directly to the defect site.

The Inside-Out Technique:

To perform the inside-out technique for the repair of a medial meniscus, a posteromedial skin incision is necessary in order to assist in the identification and capture of the needles during the later stage inside-out suturing procedure and to ensure the proper identification and protection of neurovascular structures. The incision should be approximately 3-5 cm in length, ⅓ above and ⅔ below the joint line, and parallel to the posterior margin of the medial collateral ligament. Once the incision is made, attempts should be made to identify and isolate the infrapatellar branch of the saphenous nerve. The incision is deepened with blunt dissection until the capsule is reached. A tissue protection spoon retractor is placed in the incision as deeply as possible; this instrument will help to protect the neurovascular structures while the suture needles are passed from the inside of the joint to the medial outer aspects of the knee.

To suture the meniscus, CHONDROGEL™ is applied to the open defect in an amount approximated to fill the open defect; any excess CHONDROGEL™ will be displaced out of the meniscus. By using conventional inside-out suturing cannulae, or the SHARPSHOOTER® Tissue Repair System, the defect is sutured to the remaining meniscus with size 2-0 braided polyester. The first horizontal suture is either placed in the anterior or posterior aspect of the meniscus defect. Using a double-armed suture, the first arm is typically placed into the native meniscus remnant and the second arm into the torn meniscus. Vertical mattress sutures are used throughout the entire length of the implant. The sutures should be placed midway between the inner and outer margins of the meniscus. This technique provides the greatest resistance to the sutures cutting through the implant. Sutures should be placed every 4-5 mm.

When using the SHARPSHOOTER® Tissue Repair System it is helpful to advance the needle of the second arm about 2 mm out of the cannula before placing the stitch. With the advanced needle, the tom meniscus can be picked up approximately mid-width. With the application of slight posterior pressure the proper positioning of the meniscus is assured before advancing the needle.

If a suture is improperly placed, it should be removed and reinserted. The tissue protection spoon retractor should be used at the medial incision to facilitate identification and capture of the needles. Care should be taken to avoid damage to the surrounding neurovascular structures. After placement of all sutures, they should be tied over the capsule through the medial skin incision while the repair remains under visual control of the arthroscope. When tying the sutures, the tension should allow apposition of the defect, but the sutures should not be tied tightly like a meniscus repair suture. The direct visualization will help to assure that the sutures are tied with correct tension.

### EXAMPLE 7

### Use of the Composition in Microfracture Surgical Technique

Microfracture is a surgical technique developed to treat chondral defects, i.e., damaged areas of the articular cartilage of the knee. It is commonly performed in patients with full thickness damage to the articular cartilage that proceeds to the bone. The procedure allows bone marrow and blood to enter the site, which will then result in tissue regeneration on the chondral surface. There are two problems with the microfracture technique to date: trying to keep the formed clot in place and the quality of the cartilage that regenerates. Once the microfracture technique is performed, it often fails because the established marrow clot dislodges into the joint.

Another important aspect of the microfracture procedure that warrants careful consideration is the quality of the cartilage that requires regeneration. Ideally, the new cartilage that forms should not be infiltrated with blood vessels; if blood vessels infiltrate the cartilage, an inferior fibrocartilage will result.

To prevent bone marrow, blood, and associated growth factors from leaving the site of the microfracture and also to prevent angiogenesis, CHONDROGEL™ alone or CHONDROGEL™ loaded with paclitaxel or other angiogenesis inhibitors should be applied to the microfracture site as explained in the following animal model procedure.

Pigs are anesthetized using standard techniques and maintained on anesthesia. One knee joint is aseptically prepared and using appropriate surgical techniques to allow recovery of the animal. A portion of the articular cartilage is exposed and removed. The amount of tissue removed is similar to that found in human defects. The size of the defect should be the same in each animal. Multiple holes, or microfractures, are then made through the cartilage into the bone approximately 3-4 mm apart. Bone marrow and blood will well up to fill the holes and flow into the defect to form a clot. Following this procedure, the area is left alone (control) or CHONDROGEL™ and/or CHONDROGEL™ plus paclitaxel at various concentrations in separate animal groups is applied to the area. The CHONDROGEL™ is applied to the general site of the defect above the series of holes and clot. The amount of material applied will depend on the area coverage of the holes and may range from 0.05 mL to approximately 0.75 mL. The CHONDROGEL™ can either be applied according to Example 2 or it can be applied according to Example 3 in which the PEG/methylated collagen is applied to the site and once it is deemed to be where desired, the basic buffer is dripped on top of the PEG/methylated collagen composition to produce the crosslinked matrix. Concentrations of paclitaxel may range between 0.1 mg/mL to about 1.0 mg/mL.

Following the procedure, the area is cleaned, and the tissues are sutured in layers using standard surgical technique. The procedure may also be done arthroscopically. The animals are then recovered. At various time intervals (e.g., 2 weeks, 1 month, 2 months, 3 months, 6 months, and possibly longer), the animals are sacrificed, and the microfracture site exposed and visually assessed for healing. Sections are then taken for histological analysis to assess the extent of healing and to differentiate the production of normal cartilage from fibrocartilage. The groups showing the best healing with no fibrocartilage indicate the appropriate dose of paclitaxel to use for clinical assessment.

To test the procedure clinically, the microfracture procedure is done arthroscopically in patients who are chosen with a full thickness defect in the knee cartilage, which is a defect that extends down to the bone. After the diagnosis of a chondral defect is made through an appropriate clinical exam and investigation, it is reasonable to surgically intervene. Three standard portals are made about the knee, to accommodate the inflow canula, the arthroscope, and the working instruments. A tourniquet is not used during the microfracture procedure. A thorough inspection of all the geographic regions of the knee is carried out through the arthroscope (including suprapatellar pouch the medial and lateral gutters, the patellofermoral joint, the intercondylar notch and its contents, and the medial and lateral compartments including the posterior horns of both menisci). If other procedures are being carried out during the same surgery (e.g., ligament reconstruction), the microfracture technique should be the last procedure.

During the procedure, carefully assess the full thickness of the cartilage lesion and debride the exposed bone of all remaining unstable cartilage; this can be done with a hand held curved curette and a full radius resector. Along with any unstable cartilage, any calcified cartilage layer that remains as a cap to any lesions must also be removed. Care must be taken to preserve the subchondral plate by not debriding too deeply. The prepared surgical lesion should have stable perpendicular edges of healthy well-attached viable cartilage surrounding the defect, which provides a reservoir to hold the marrow “super clot.”

An arthroscopic awl is used to make multiple holes or “microfractures” in the exposed subchondral bone plate. The awl used should have an angle that permits the top to be perpendicular to the bone as it is advanced, typically 30 to 45 degrees, with 90 degrees used for the patella. The holes are made as close together as possible, but not so close that one breaks into another damaging the subchondral bone. Usually the holes will be 3 to 4 mm apart. The appropriate depth will generate fat droplets from the marrow cavity, often when a depth of 2 to 4 mm is reached. Microfractures are first started along the periphery, then along the stable cartilage, and then towards the center of the lesion. Once the surface is prepared, the arthroscopic irrigation fluid pump pressure is reduced; the preparation is adequate when marrow is emanating from all the mircrofracture holes. CHONDROGEL™ is then applied over the lesion, sealing in the marrow-rich clot and all its elements. Intraarticular drains should not be used.

In the case of chronic degenerative chondral lesions, the bone is often eburnated and sclerosed, making it difficult to do adequate microfracture procedure. The same process is carried out as above, but often the rim of cartilage is too thin and is not adequate hold the marrow clot. In this case, the microfracture technique is again carried out as described above with CHONDROGEL™ applied to stabilize the marrow clot in place after the procedure.

CHONDROGEL™ with or without paclitaxel is placed over the microfracture areas. The optimal amount of CHONDROGEL™ and concentrations of paclitaxel used will have been determined from preclinical studies. The marrow-rich clot is the basis for the new tissue formation; the clot eventually matures into firm repair tissue that becomes smooth and durable. Since the maturing process is gradual, it may take two to six months after the procedure for the patient to experience improvement in the pain and function of the knee. Thus, a second look may be performed to assess healing at a prescribed interval between 2 and 6 months.

### EXAMPLE 8

### Screening Assay for Assessing the Effect of Cyclosporin a on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. Cyclosporin A is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and cyclosporin A are incubated at 37° C. for 72 hours.

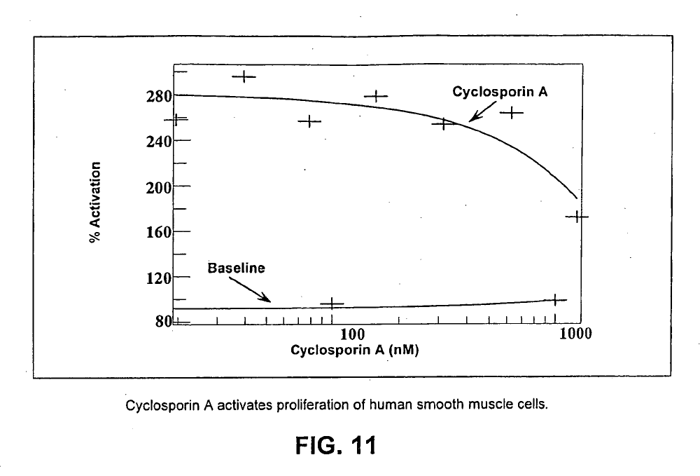

To terminate the assay, the media is removed by gentle aspiration. A 1/400 dilution of CYQUANT® 400×OR dye indicator (Molecular Probes, Eugene, Oreg.) is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and ˜˜520 nm emission maxima. Activation of proliferation is determined by taking the average of triplicate wells and comparing average relative fluorescence units to the DMSO control. See, FIG. 11 and the following reference: IN VITRO TOXICOL. 3: 219 (1990); BIOTECH. HISTOCHEM. 68: 29 (1993); ANAL. BIOCHEM. 213: 426 (1993).

### EXAMPLE 9

### Screening Assay for Assessing the Effect of Dexamethasone on Cell Proliferation

An in vitro assay is described for determining whether a substance stimulates cell (fibroblast) proliferation. Fibroblasts at 70-90% confluency are trypsinized, replated at 600 cells/well in media in 96-well plates and allowed to attachment overnight. Dexamethasone is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and dexamethasone are incubated at 37° C. for 72 hours.

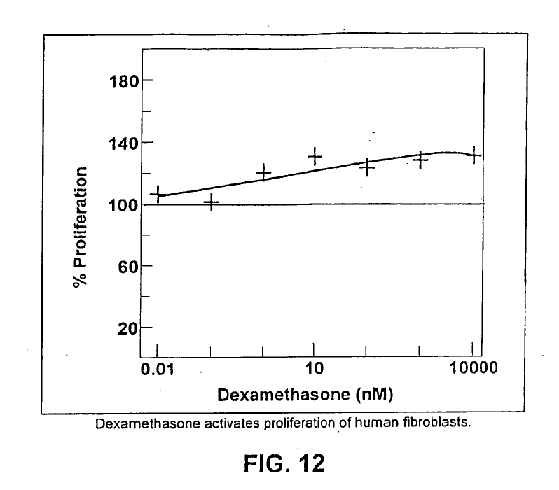

To terminate the assay, the media is removed by gentle aspiration. A 1/400 dilution of CYQUANT® 400×GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜˜480 nm excitation wavelength and ˜˜520 nm emission maxima. Activation of proliferation is determined by taking the average of triplicate wells and comparing average relative fluorescence units to the DMSO control. See, FIG. 12 and the following references: IN VITRO TOXICOL. 3: 219 (1990); BIOTECH. HISTOCHEM. 68: 29 (1993); ANAL. BIOCHEM. 213:426 (1993).

### EXAMPLE 10

### Screening Assay for Assessing the Effect of

### All-Trans Retinoic Acid on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. All-trans retinoic acid is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and all-trans retinoic acid are incubated at 37° C. for 72 hours.

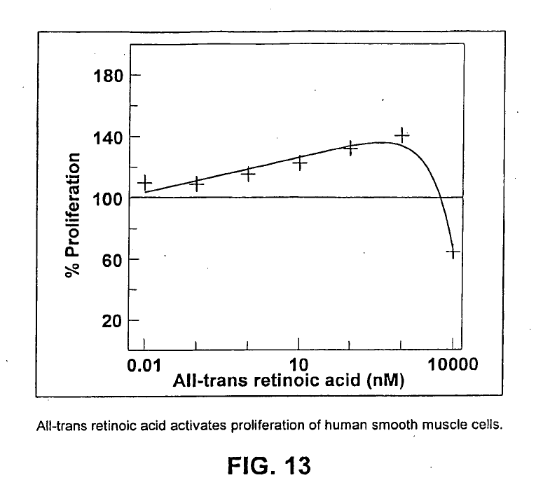

To terminate the assay, the media is removed by gentle aspiration. A 1/400 dilution of CYQUANT® 400×GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜480 nm excitation wavelength and 520 nm emission maxima. Activation of proliferation is determined by taking the average of triplicate wells and comparing average relative fluorescence units to the DMSO control. See, FIG. 13 and the following reference: IN VITRO TOXICOL. 3: 219 (1990); BIOTECH. HISTOCHEM. 68: 29 (1993); ANAL. BIOCHEM. 213: 426 (1993).

### EXAMPLE 11

### Screening Assay for Assessing the Effect of Isotretinoin on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. Isotretinoin is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing smooth muscle cells and isotretinoin are incubated at 37° C. for 72 hours.

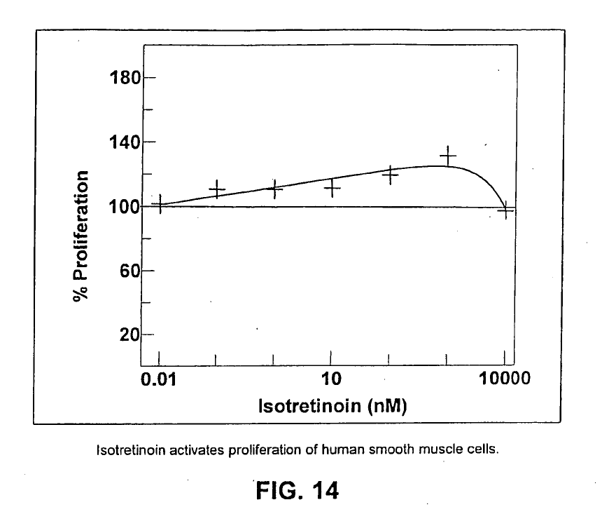

To terminate the assay, the media is removed by gentle aspiration. A 1/400 dilution of CYQUANT® 400×GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜˜480 nm excitation wavelength and ˜˜520 nm emission maxima. Activation of proliferation is determined by taking the average of triplicate wells and comparing average relative fluorescence units to the DMSO control. See, FIG. 14 and the following references: IN VITRO TOXICOL. 3:219 (1990); BIOTECH. HISTOCHEM. 68: 29 (1993); ANAL. BIOCHEM. 213:426 (1993).

### EXAMPLE 12

### Screening Assay for Assessing The Effect of 17-B-Estradiol on Cell Proliferation

An in vitro assay is described for determining whether a substance stimulates cell (fibroblast) proliferation. Fibroblasts at 70-90% confluency are trypsinized, replated at 600 cells/well in media in 96-well plates, and allowed to attachment overnight. 17-β-estradiol is prepared in DMSO at a concentration of 10⁻² M and diluted 10-fold to give a range of stock concentrations (10⁻⁸ M to 10⁻² M). Drug dilutions are diluted 1/1000 in media and added to cells to give a total volume of 200 μL/well. Each drug concentration is tested in triplicate wells. Plates containing fibroblasts and 17-β-estradiol are incubated at 37° C. for 72 hours.

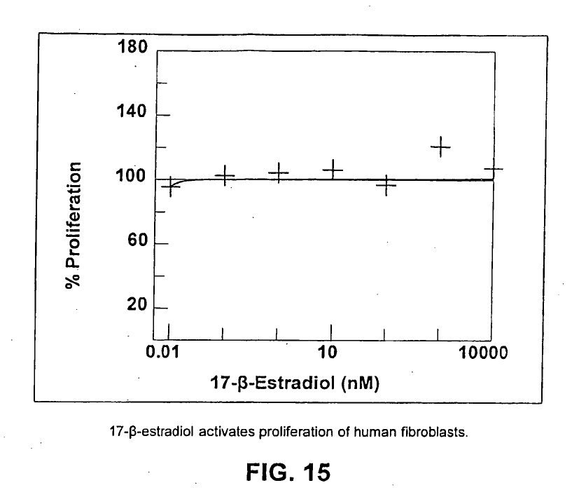

To terminate the assay, the media is removed by gentle aspiration. A 1/400 dilution of CYQUANT® 400×GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜˜480 nm excitation wavelength and ˜˜520 nm emission maxima. Activation of proliferation is determined by taking the average of triplicate wells and comparing average relative fluorescence units to the DMSO control. See FIG. 15 and the following references: IN VITRO TOXICOL. 3: 219 (1990); BIOTECH. HISTOCHEM. 68: 29 (1993); ANAL. BIOCHEM. 213: 426 (1993).

### EXAMPLE 13

### Screening Assay for Assessing the Effect of

### 1α,25-Dihydroxy-Vitamin D₃ on Cell Proliferation

An in vitro assay is described for determining whether a substance stimulates cell (fibroblast) proliferation. Smooth muscle cells at 70-90% confluency are trypsinized, replated at 600 cells/well in media in 96-well plates and allowed to attachment overnight. 1α,25-Dihydroxy-vitamin D₃ is prepared in DMSO at a concentration of 10⁻² M and diluted 10-fold to give a range of stock concentrations (10⁻⁸ to 10⁻² M). Drug dilutions are diluted 1/1000 in media and added to cells to give a total volume of 200 μL/well. Each drug concentration is tested in triplicate wells. Plates containing smooth muscle cells and 1α,25-Dihydroxy-vitamin D₃ are incubated at 37° C. for 72 hours.

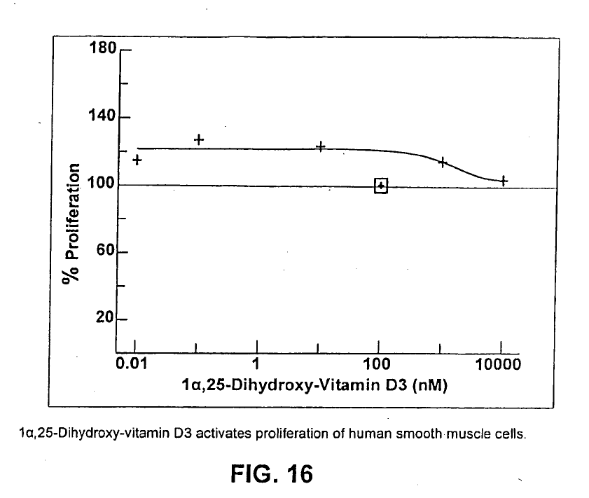

To terminate the assay, the media is removed by gentle aspiration. A 1/400 dilution of CYQUANT® 400× GR dye indicator is added to 1× Cell Lysis buffer, and 200 μL of the mixture is added to the wells of the plate. Plates are incubated at room temperature, protected from light for 3-5 minutes. Fluorescence is read in a fluorescence microplate reader at ˜˜480 nm excitation wavelength and ˜˜520 nm emission maxima. Activation of proliferation is determined by taking the average of triplicate wells and comparing average relative fluorescence units to the DMSO control. See, FIG. 16 and the following references: IN VITRO TOXICOL. 3: 219 (1990); BIOTECH. HISTOCHEM. 68: 29(1993); and ANAL. BIOCHEM. 213: 426 (1993).

### EXAMPLE 14

### Screening Assay for Assessing the Effect of PDGF on Smooth Muscle Cell Migration

An in vitro assay is described for determining whether a substance stimulates cell (fibroblast) migration. Primary human smooth muscle cells are starved of serum in smooth muscle cell basal media containing insulin and human basic fibroblast growth factor (bFGF) for 16 hours prior to the assay. For the migration assay, cells are trypsinized to remove cells from flasks, washed with migration media and diluted to a concentration of 2-2.5×10⁵ cells/mL in migration media. Migration media consists of phenol red free Dulbecco's Modified Eagle Medium (DMEM) containing 0.35% human serum albumin. A 100 μL volume of smooth muscle cells (approximately 20,000-25,000 cells) is added to the top of a Boyden chamber assembly (Chemicon QCM Chemotaxis 96-well migration plate). To the bottom wells, the chemotactic agent, recombinant human platelet derived growth factor (rhPDGF-BB) is added at a concentration of 10 ng/mL in a total volume of 150 μL. Paclitaxel is prepared in DMSO at a concentration of 10⁻² M and serially diluted 10-fold to give a range of stock concentrations (10⁻⁸ M to 10⁻² M). Paclitaxel is added to cells by directly adding paclitaxel DMSO stock solutions, prepared earlier, at a 1/1000 dilution, to the cells in the top chamber. Plates are incubated for 4 hours to allow cell migration.

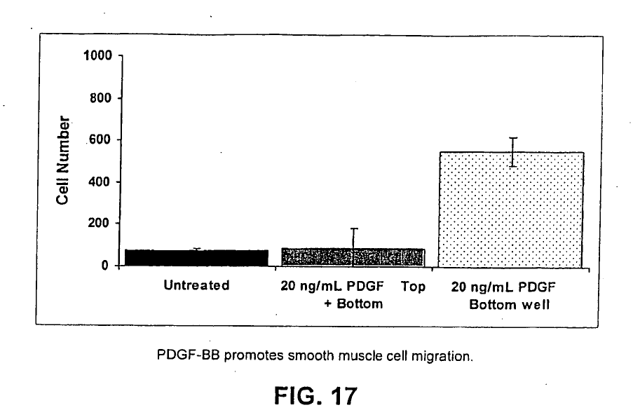

At the end of the 4-hour period, cells in the top chamber are discarded and the smooth muscle cells attached to the underside of the filter are detached for 30 minutes at 37° C. in Cell Detachment Solution (Chemicon). Dislodged cells are lysed in lysis buffer containing the DNA binding CYQUANT® OR dye and incubated at room temperature for 15 minutes. Fluorescence is read in a fluorescence microplate reader at ˜480 nm excitation wavelength and ˜520 nm emission maxima. Relative fluorescence units from triplicate wells are averaged after subtracting background fluorescence (control chamber without chemoattractant) and average number of cells migrating is obtained from a standard curve of smooth muscle cells serially diluted from 25,000 cells/well down to 98 cells/well. Inhibitory concentration of 50% (IC₅₀) is determined by comparing the average number of cells migrating in the presence of paclitaxel to the positive control (smooth muscle cell chemotaxis in response to rhPDGF-BB). See, FIG. 17 and the following references: BIOTECHNIQUES 29: 81 (2000) and J. IMMUNOL METHODS 254: 85 (2001).

### EXAMPLE 15

### Collagen Synthesis Assay

An in vitro assay is described for determining whether a substance promotes deposition of extracellular matrix (ECM). Normal human dermal fibroblasts were trypzanized, then re-plated in medium containing ascorbic acid-2-phosphate at 150,000 cells per well in a 12-well plate. The cells were cultured at 37° C. and 5% CO₂ for 2-3 weeks with media changes every three days so that they formed a 3-D matrix of cells and collagen. After 14-21 days of culture, the medium was replaced with serum free medium and the cells allowed to rest for 24 hours.

Drug was diluted in DMSO at 10⁻²M and then diluted 10 fold to give a range of stock concentrations from 10⁻²M to 10⁻⁸M. Drug was then diluted 1000 times in fresh serum free medium and added to the wells in a total volume of 3 ml per well. The plate(s) were then incubated for 72 hrs at 37° C. After 72 hrs the media was removed from the wells and put into microcentrifuge tubes and frozen at −20° C. until assayed.

The amount of collagen synthesized was measured using a Procollagen Type 1 C-Peptide (PIP) EIA (enzyme immunoassay) kit (Takara Bio Inc., Shiga, Japan), where the amount of collagen produced is stoichiometrically represented by the amount of pro-peptide cleaved from the collagen when it is secreted. Anti-PIP monoclonal antibodies are immobilized on an ELISA (enzyme-linked immonsorbant assay) plate, the samples added and then a second PIP monoclonal antibody conjugated to horseradish peroxidase is added to the wells and incubated. Following incubation the wells are washed, a substrate solution is added and the absorbance measured in a plate reader at 450 nm and compared to a standard curve of PIP (ng/ml).

### EXAMPLE 16

### Meniscal Tear Animal Model

This example describes an in vivo method to create a meniscal tear in a porcine joint and application of a formulation to assess for healing. Domestic farm pigs weighing 30 to 40 kg are anesthetized with an IM injection of ketamin/xylazine and atropine. Anaesthesia is maintained by inhalation with isoflurane. The skin over one knee joint is cleaned and sterilized with an antiseptic solution. The knee capsule is exposed using a lateral skin incision. The capsule is entered taking care not to injure the collateral and cruciate ligaments and the articular cartilage. A full thickness longitudinal incision is created in the avascular part of the lateral meniscus. Approximately 0.2-0.5 ml of the sterile, biocompatible formulation is applied into the incision. The knee capsule, the surrounding tissue and the skin is closed in layers. The animal is recovered and returned to the holding facility. Analgesics and antibiotics are given at the end of the surgical procedure. Walking behavior is monitored daily. Analgesics and anti-inflammatory drugs may be given to control swelling and pain.

The animals are sacrificed 4-8 weeks after the procedure. The animals are anesthetised and euthanized with an IV injection of Euthanyl. The meniscus is be harvested for histology processing in order to assess healing after the different treatment regimens.

### EXAMPLE 17

### Preparation of Drug Loaded Microspheres by Spray Drying

3.6 grams of methoxy poly(ethylene glycol 5000)-block-(poly (DL-lactide) (65:35 MePEG5000:PDLLA) was dissolved in 200 mL methylene chloride. 400 mg of paclitaxel (PTX)) was added and the resulting solution was spray dried. Conditions: inlet temperature 50° C., outlet temperature <39° C., aspirator 100%, flow rate 700 mL/hr. The collected microspheres were dried under vacuum at room temperature overnight to produce uniform spherical particles having size ranges of less than about 10 microns (typically about 0.5 to about 2 microns).

### EXAMPLE 18

### PTX-Loaded Microspheres (<10 Micron) by the W/O/W Emulsion Process

100 ml of freshly prepared 10% polyvinyl alcohol (PVA) solution was added into a 600 mL beaker. The acidified PVA solution was stirred at 2000 rpm for 30 minutes. Meanwhile, a solution of 80 mg PTX and 800 mg MePEG5000-PDLLA (65:35) in 20 mL dichloromethane was prepared. The polymer/dichloromethane solution was added dropwise to the PVA solution while stirring at 2000 rpm with a Fisher Dyna-Mix stirrer. After the addition was complete, the solution was allowed to stir for an additional 45 minutes. The microsphere solution was transferred to several disposable graduated polypropylene conical centrifuge tubes, washed with deionized water, and centrifuged at 2600 rpm for 10 minutes. The aqueous layer was decanted and the washing, centrifuging, and decanting was repeated 3 times. The combined, washed microspheres were freeze-dried and vacuum dried to remove any excess water.

### EXAMPLE 19

### PTX-Containing Micelles

MePEG2000 (41 g) and MePEG2000-PDLLA (60:40) (410 g) were combined in a vessel and heated to 75° C. with stirring. After the polymers were completely melted and mixed, the temperature was decreased to 55° C. Meanwhile, a PTX solution in tetrahydrofuran (46 g/200 mL) was prepared and poured into the polymer solution under constant stirring. Stirring was continued for an additional hour. The PTX-containing micelles were dried at 50° C. under vacuum to remove solvent and were ground on a 2 mm mesh screen after cooling.

### EXAMPLE 20

### PTX-Loaded Microspheres (<10 Micron) by the W/O/W Emulsion Process

100 ml of freshly prepared 10% PVA solution was added into a 600 mL beaker. The acidified PVA solution was stirred at 2000 rpm for 30 minutes. Meanwhile, a solution of 80 mg PTX and 800 mg poly(lactic-co-glycolic acid (PLGA) (50:50, IV=0.54) in 20 ml dichloromethane was prepared. The polymer/dichloromethane solution was added dropwise to the PVA solution while stirring at 2000 rpm with a Fisher Dyna-Mix stirrer. After addition was complete, the solution was allowed to stir for an additional 45 minutes. The microsphere solution was transferred to several disposable graduated polypropylene conical centrifuge tubes, washed with deionized water, and centrifuged at 2600 rpm for 10 minutes. The aqueous layer was decanted and the washing, centrifuging, and decanting was repeated 3 times. The combined, washed microspheres were freeze-dried and vacuum dried to remove any excess water.

### EXAMPLE 21

### Gelation of PTX-Loaded NHS-PEG, HS-PEG, and MC

A PTX-loaded crosslinked hydrogel was formed from an acidic solution of NHS-PEG, HS-PEG, and MC for immediatel gelation according to the following procedure.

A 1 mL syringe (syringe 1) equipped with a luer-lock mixing connector was filled with a mixture of PEG-SG4 (50 mg), PEG-SH4 (50 mg), and 10% PTX-loaded MePEG5000-PDLLA (65:35) microspheres prepared by spray drying (0.5 mg, 2 mg, 10 mg, or 25 mg). A 1 mL capped syringe (syringe 2) was filled with 0.50 mL of 22 mg/mL MC (pH 3-4). A 1 mL capped syringe (syringe 3) was filled with 0.25 mL 0.12 M monobasic sodium phosphate and 0.2 M sodium carbonate (pH 9.7) buffer. The contents of syringe 1 and syringe 2 were mixed through a mixing connector by repeatedly transferring the contents from one syringe to the other. After complete mixing, all of the formulation was pushed into one of the syringes which was then attached to a γ-shaped dual syringe connector. Syringe 3 was connected to the remaining connector. The material was applied by simultaneously depressing the plungers of both syringes. The gelation occurred within seconds to form a strong hydrogel that adheres well to the tissue and swells in a controlled fashion in saline solution.

The process was repeated using the PTX-loaded microspheres that were prepared in Examples 18 and 20.

### EXAMPLE 22

### Gelation of PTX-Loaded NHS-PEG HS-PEG and MC on a Tissue Sample

A PTX-loaded crosslinked hydrogel was formed from an acidic solution of NHS-PEG, HS-PEG, and MC for gelation on a tissue sample according to the following procedure.

A 1 ml syringe (syringe 1) equipped with a luer-lock mixing connector was filled with a mixture of PEG-SG4 (50 mg), PEG-SH4 (50 mg), and 10% PTX-loaded MePEG000-PDLLA (65:35) microspheres prepared by spray drying (0.5 mg, 2 mg, 10 mg, or 25 mg). A 1 mL capped syringe (syringe 2) was filled with 0.50 mL of 22 mg/mL MC (pH 3-4). A 1 mL capped syringe (syringe 3) was filled with 0.5 mL 0.12 M monobasic sodium phosphate and 0.2 M sodium carbonate (pH 9.7) buffer. The contents of syringe 1 and syringe 2 were mixed through a mixing connector by repeatedly transferring the contents from one syringe to the other. After complete mixing, all of the formulation was pushed into one of the syringes. The material was then applied to the desired location of a tissue sample. A spatula was used to manipulate the final shape of the applied material on the tissue. The basic buffer (syringe 3) was dripped over the surface of the applied material. After 30 minutes a strong hydrogel that adhered well to the tissue sample was obtained. The process was repeated using the PTX-loaded microspheres that were prepared in Examples 18 and 20.

### EXAMPLE 23

### Incorporation of PTX-Loaded Micelles into the Composition

A 1 mL syringe (syringe 1) equipped with a luer-lock mixing connector is filled with a mixture of PEG-SG4 (50 mg) and PEG-SH4 (50 mg). 5 mg, 10 mg, and 25 mg of the micellar PTX described in Example 19 is weighed into a 1 mL syringe (syringe 2). A syringe containing 0.50 mL of 22 mg/mL MC (pH 3-4) was attached to syringe 2 via a syringe connector. The contents of the 2 syringes were mixed by transferring the contents from one syringe to the other. The contents were transferred to one of the syringes which was then connected to syringe 1. The mixing process was repeated and the contents were transferred to one of the syringes. The material was then applied to the desired location of a tissue sample. A spatula was used to manipulate the final shape of the applied material on the tissue sample. The basic buffer (syringe 3) was dripped over the surface of the applied material. After 30 minutes a strong hydrogel that adhered well to the tissue sample was obtained.

### EXAMPLE 24

### Inhibition of Angiogenesis by Paclitaxel and Mitoxantrone

Chick Chorioallantoic Membrane (“CAM”) Assays

Fertilized, domestic chick embryos were incubated for 3 days prior to culturing. In this procedure, the shell located over the air space was removed and the outer egg membrane gently peeled away to expose the CAM membrane. The egg opening was covered with parafilm and the eggs placed into an incubator at 90% relative humidity and 3% CO₂ and incubated for 2 days.

Paclitaxel (Sigma, St. Louis, Mich.) was mixed at concentrations of 0.25, 0.5, 1, 5, 10, 30 μg per 10 μL aliquot of 0.5% aqueous methylcellulose, while mitoxantrone was mixed with 0.5% aqueous methylcellulose at concentrations of 1, 5, 10 μg per 10 μL aliquot. Since both paclitaxel and mitoxantrone are insoluble in water, the compounds were dissolved in dimethylsulfoxide (DMSO). 10 μL aliquots of this solution were dried on parafilm for 4-6 hours forming disks 2 mm in diameter. The dried disks containing paclitaxel or mitoxantrone were then carefully placed at the growing edge of each CAM at day 6 of incubation. Controls were obtained by placing DMSO-containing methylcellulose disks on the CAMs over the same time course. After a 2 day exposure (day 8 of incubation) the vasculature was examined with the aid of a stereomicroscope. Liposyn II, a white opaque solution, was injected into the CAM to increase the visibility of the vascular details. The vasculature of unstained, living embryos were imaged using a Zeiss stereomicroscope which was interfaced with a video camera (Dage-MTI Inc., Michigan City, Ind.). These video signals were then displayed at 160× magnification and captured using an image analysis system (Vidas, Kontron; Etching, Germany). Image negatives were then made on a graphics recorder (Model 3000; Matrix Instruments, Orangeburg, N.Y.).

For the following experiments, shell-less culturing methods were used where the egg contents were emptied by removing the shell located around the air space. The interior shell membrane was severed and the opposite end of the shell was perforated to allow the contents of the egg to gently slide out. The egg contents were emptied into round-bottom sterilized glass bowls and covered with Petri dish covers. Procedures were then followed as described above with respect to drug preparation and incubation. The membranes of the 8-day-old shell-less embryo were flooded with 2% glutaraldehyde in 0.1M sodium cacodylate buffer; additional fixative was injected under the CAM. After 10 minutes in situ, the CAM was removed and placed into fresh fixative for 2 hours at room temperature. The tissue was then washed overnight in cacodylate buffer containing 6% sucrose. The areas of interest were postfixed in 1% osmium tetroxide for 1.5 hours at 4° C. The tissues were then dehydrated in a graded series of ethanols, solvent exchanged with propylene oxide, and embedded in Spurr resin. Thin sections were cut with a diamond knife, placed on copper grids, stained, and examined in a Joel 1200EX electron microscope. Similarly, 0.5 mm sections were cut and stained with toluene blue for light microscopy.

At day 11 of development, chick embryos were used for the corrosion casting technique. Mercox resin (Ted Pella, Inc., Redding, Calif.) was injected into the CAM vasculature using a 30-gauge hypodermic needle. The casting material consisted of 2.5 grams of Mercox CL-2B polymer and 0.05 grams of catalyst (55% benzoyl peroxide) having a 5-minute polymerization time. After injection, the plastic was allowed to sit in situ for an hour at room temperature and then overnight in an oven at 65° C. The CAM was then placed in 50% aqueous solution of sodium hydroxide to digest all organic components. The plastic casts were washed extensively in distilled water, air-dried, coated with gold/palladium, and viewed with the Philips 501B scanning electron microscope.

Results of the assay were as follows. At day 6 of incubation, the embryo was centrally positioned to a radially expanding network of blood vessels; the CAM developed adjacent to the embryo. These growing vessels lie close to the surface and are readily visible making this system an idealized model for the study of angiogenesis. Living, unstained capillary networks of the CAM may be imaged non-invasively with a stereomicroscope.

Transverse sections through the CAM show an outer ectoderm consisting of a double cell layer, a broader mesodermal layer containing capillaries which lie subjacent to the ectoderm, adventitial cells, and an inner, single endodermal cell layer. At the electron microscopic level, the typical structural details of the CAM capillaries are demonstrated. Typically, these vessels lie in close association with the inner cell layer of ectoderm.

After 48 hours exposure to paclitaxel at concentrations of 0.25, 0.5, 1, 5, 10, or 30 μg or mitoxantrone at concentrations of 1, 5, 10 μg each CAM was examined under living conditions with a stereomicroscope equipped with a video/computer interface in order to evaluate the effects on angiogenesis. This imaging setup was used at a magnification of 160× which permitted the direct visualization of blood cells within the capillaries; thereby blood flow in areas of interest may be easily assessed and recorded. For this study, the inhibition of angiogenesis was defined as an area of the CAM (measuring 2-6 mm in diameter) lacking a capillary network and vascular blood flow. Throughout the experiments, avascular zones were assessed on a 4-point avascular gradient as shown in Table 3. In Table 3, the scale represents the degree of overall inhibition with maximal inhibition represented as a 3 on the avascular gradient scale. Both paclitaxel and mitoxantrone were very consistent and induced a maximal avascular zone (6 mm in diameter or a 3 on the avascular gradient scale) within 48 hours depending on its concentration.

TABLE 3
AVASCULAR GRADIENT

0 -- normal vascularity
1 -- lacking some microvascular movement
2*-- small avascular zone approximately 2 mm in diameter
3*-- avascularity extending beyond the disk (6 mm in diameter)
*indicates a positive antiangiogenesis response

The dose-dependent, experimental data of the effects of paclitaxel at different concentrations are shown in Table 4.

TABLE 4

| | | CONCEN- | | INHIBI- |
|---|---|---|---|---|
| AGENT | DELIVERY VEHICLE | TRATION | | TION/N |
| paclitaxel | methylcellulose (10 μl) | 0.25 | μg | 2/11 |
| | methylcellulose (10 μl) | 0.5 | μg | 6/11 |
| | methylcellulose (10 μl) | 1 | μg | 6/15 |
| | methylcellulose (10 μl) | 5 | μg | 20/27 |
| | methylcellulose (10 μl) | 10 | μg | 16/21 |
| | methylcellulose (10 μl) | 30 | μg | 31/31 |
| mitoxantrone | methylcellulose (10 μl) | 1 | μg | 18/24 |
| | methylcellulose (10 μl) | 5 | μg | 21/24 |
| | methylcellulose (10 μl) | 10 | μg | 18/22 |

Typical paclitaxel and mitoxantrone treated CAMs are also shown with the transparent methylcellulose disk centrally positioned over the avascular zone measuring 6 mm in diameter. At a slightly higher magnification, the periphery of such avascular zones is clearly evident; the surrounding functional vessels were often redirected away from the source of paclitaxel. Such angular redirecting of blood flow was never observed under normal conditions. Another feature of the effects of paclitaxel was the formation of blood islands within the avascular zone representing the aggregation of blood cells.

In summary, this study demonstrated that 48 hours after both paclitaxel and mitoxantrone application to the CAM, angiogenesis was inhibited. The blood vessel inhibition formed an avascular zone which was represented by three transitional phases of paclitaxel's effect. The central, most affected area of the avascular zone contained disrupted capillaries with extravasated red blood cells; this indicated that intercellular junctions between endothelial cells were absent. The cells of the endoderm and ectoderm maintained their intercellular junctions and therefore these germ layers remained intact; however, they were slightly thickened. As the normal vascular area was approached, the blood vessels retained their junctional complexes and therefore also remained intact. At the periphery of the paclitaxel-treated zone, further blood vessel growth was inhibited which was evident by the typical redirecting or “elbowing” effect of the blood vessels.

1-217. (canceled)

218. A method of repairing damaged cartilage tissue in a patient comprising placing into contact with the damaged cartilage tissue a composition comprising components (i), (ii), and (iii) or a partial reaction product of components (i), (ii), and (iii), wherein components (i), (ii), and (iii) are comprised of:
(i) a first hydrophilic polymer;
(ii) a crosslinkable component A having m nucleophilic groups, wherein m≧2; and
(iii) a crosslinkable component B having n electrophilic groups capable of reaction with the m nucleophilic groups to form covalent bonds, wherein n≧2 and m+n>4,
wherein each of components A and B is biocompatible and nonimmunogenic, at least one of components A and B is a second hydrophilic polymer, and reaction of components (i), (ii), and (iii) results in a biocompatible, nonimmunogenic, crosslinked matrix.

219. The method of claim 218, wherein the damaged cartilage tissue is selected from the group consisting of articular cartilage tissue, a meniscus, or labrum.

220. The method of claim 218, wherein the first hydrophilic polymer is selected from a synthetic hydrophilic polymer and a naturally occurring hydrophilic polymer.

221. The method of claim 220, wherein the naturally occurring hydrophilic polymer is selected from the group consisting of proteins, peptides, polysaccharides, lipids and derivatives thereof.

222. The method of claim 221, wherein the protein is a collagen.

223. The method of claim 222, wherein the collagen is selected from the group consisting of nonfibrillar collagen, fibrillar collagen, and a mixture of nonfibrillar collagen and fibrillar collagen.

224. The method of claim 223, wherein the nonfibrillar collagen is selected from the group consisting of methylated collagen, type IV collagen, type VI collagen, and type VII collagen.

225. The method of claim 218, wherein the second hydrophilic polymer is selected from the group consisting of polyalkeleneoxides, polyurethanes, polyesters, polyethers, polythioethers, polyamides, and derivatives, copolymers, and combinations thereof.

226. The method of claim 218, wherein components A and B each comprise a polyalkyleneoxide.

227. The method of claim 226, wherein the polyalkyleneoxide is a poly(ethylene glycol).

228. The method of claim 218, wherein components A and B are the same.

229. The method of claim 218, wherein components A and B are in admixture.

230. The method of claim 229, wherein the admixture is in a solid form.

231. The method of claim 218, wherein component A has the structural formula (I) and component B has the structural formula (II)
R¹(-[Q¹]_{q}-X)ₘ (I)
R²(-[Q²]ᵣ-Y)ₙ (II)
wherein:
R¹ and R² are independently selected from the group consisting of C₂ to C₁₄ hydrocarbyl, heteroatom-containing C₂ to C₁₄ hydrocarbyl, hydrophilic polymers, and hydrophobic polymers;
X represents one of the m nucleophilic groups of component A;
Y represents one of the n electrophilic groups of component B;
Q¹ and Q² are linking groups; and
q and r are independently zero or 1.

232. The method of claim 231, wherein at least one of R¹ and R² is a synthetic hydrophilic polymer.

233. The method of claim 218, wherein the composition further comprises a third crosslinkable component C that is biocompatible and nonimmunogenic and has at least one functional group selected from
(a) nucleophilic groups capable of reacting with the electrophilic groups of component B and,
(b) electrophilic groups capable of reacting with the nucleophilic groups of component A,
wherein the total number of functional groups on component C is represented by p, such that m+n+p>5.

234. The method of claim 233, wherein component C has the structural formula (III)
R³(-[Q³]ₛFn)ₚ (III)
wherein:
R³ is selected from the group consisting of C₂ to C₁₄ hydrocarbyl, heteroatom-containing C₂ to C₁₄ hydrocarbyl, hydrophilic polymers, and hydrophobic polymers;
Fn represents a functional group on component C; and
s is zero or 1.

235. The method of claim 232, wherein the synthetic hydrophilic polymer is selected from the group consisting of: polyalkylene oxides; polyglycerols; poly(oxyalkylene)-substituted polyols; polyacrylic acid and analogues thereof; polymaleic acid; polyacrylamides; poly(olefinic alcohol)s; poly(N-vinyl lactams); polyoxazolines; polyvinylamines; and copolymers thereof.

236. The method of claim 235, wherein the synthetic hydrophilic polymer is a polyalkylene oxide selected from the group consisting of polyethylene glycol and poly(ethylene oxide)-poly(propylene oxide) copolymers.

237. The method of claim 218, wherein the nucleophilic groups on component A are selected from the group consisting of —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, and —C₅H₄N, wherein R⁴ and R⁵ are C₁-C₁₂ hydrocarbyl.

238. The method of claim 237, wherein the nucleophilic groups are selected from —NH₂ and —NHR⁴ where R⁴ is lower hydrocarbyl.

239. The method of claim 238, wherein the electrophilic groups on component B are amino-reactive groups.

240. The method of claim 237, wherein the amino-reactive groups contain an electrophilically reactive carbonyl group susceptible to nucleophilic attack by a primary or secondary amine.

241. The method of claim 237, wherein the amino-reactive groups are selected from the group consisting of carboxylic acids, carboxylic acid esters, and aldehydes.

242. The method of claim 218, wherein the composition further comprises a biologically active agent that facilitates tissue healing and regeneration.

243. The method of claim 242, wherein the biologically active agent is selected from the group consisting of an angiogenesis inhibitor, paclitaxel, enzymes, receptor antagonists, receptor agonists, hormones, growth factors, small molecules, autogenous bone marrow, antibiotics, antimicrobial agents, antibodies, cytokines, bone morphogenic proteins, and growth factors.

244. The method of claim 218, wherein (i) is methylated collagen; (ii) is pentaerythritol tetrakis[mercaptoethyl poly(oxyethylene) ether]; and (iii) is pentaerythritol tetrakis [1-(1′-oxo-5-succimidylpentanoate)-2-poly(oxyethylene) ether].

245. A method of repairing damaged cartilage tissue in a patient comprising:
(a) providing a flowable mixture comprising components (i), (ii), and (iii) or a partial reaction product of components (i), (ii), and (iii), wherein components (i), (ii), and (iii) are comprised of:
(i) a first hydrophilic polymer.
(ii) a crosslinkable component A having m nucleophilic groups, wherein m≧2, and
(iii) a crosslinkable component B having n electrophilic groups capable of reaction with the m nucleophilic groups to form covalent bonds;
(b) applying the flowable mixture to damaged cartilage tissue; and
(c) irrigating the applied mixture with an aqueous initiating buffer, to form a biocompatible, nonimmunogenic, crosslinked matrix,
wherein n≧2 and m+n>4, each of components A and B is biocompatible and nonimmunogenic, and at least one of components A and B is a second hydrophilic polymer.

246. The method of claim 245, wherein the reaction mixture is applied to the damaged cartilage tissue as a viscous liquid, partially polymerized gel, suspension, or spray.

247. The method of claim 245, wherein component A has the structural formula (I) and component B has the structural formula (II)
R¹(-[Q¹]_{q}-X)ₘ (I)
R²(-[Q²]ᵣ-Y)ₙ (II)
wherein:
R¹ and R² are independently selected from the group consisting of C₂ to C₁₄ hydrocarbyl, heteroatom-containing C₂ to C₁₄ hydrocarbyl, hydrophilic polymers, and hydrophobic polymers;
X represents one of the m nucleophilic groups of component A;
Y represents one of the n electrophilic groups of component B;
Q¹ and Q² are linking groups; and
q and r are independently zero or 1.

248. The method of claim 247, wherein at least one of R¹ and R² is a synthetic hydrophilic polymer.

249. The method of claim 245, wherein the composition further comprises a third crosslinkable component C that is biocompatible and nonimmunogenic and has at least one functional group selected from the group consisting of:
(a) nucleophilic groups capable of reacting with the electrophilic groups of component B; and
(b) electrophilic groups capable of reacting with the nucleophilic groups of component A,
wherein the total number of functional groups on component C is represented by p, such that m+n+p>5.

250. The method of claim 249, wherein component C has the structural formula (III)
R³(-[Q³]ₛ-Fn)ₚ (III)
wherein:
R³ is selected from the group consisting of C₂ to C₁₄ hydrocarbyl, heteroatom-containing C₂ to C₁₄ hydrocarbyl, hydrophilic polymers, and hydrophobic polymers;
Fn represents a functional group on component C; and
s is zero or 1.

251. The method of claim 245, wherein the nucleophilic groups on component A are selected from the group consisting of —NH₂, —NHR⁴, —N(R⁴)₂, —SH, —OH, —COOH, —C₆H₄—OH, —PH₂, —PHR⁵, —P(R⁵)₂, —NH—NH₂, —CO—NH—NH₂, and —C₅H₄N, wherein R⁴ and R⁵ are C₁-C₁₂ hydrocarbyl.

252. The method of claim 251, wherein the nucleophilic groups are selected from —NH₂ and —NHR⁴ where R⁴ is lower hydrocarbyl.

253. The method of claim 251, wherein the electrophilic groups on component B are amino-reactive groups.

254. The method of claim 253, wherein the amino-reactive groups contain an electrophilically reactive carbonyl group susceptible to nucleophilic attack by a primary or secondary amine.

255. The method of claim 218, wherein the composition further comprises a biologically active agent that facilitates tissue healing and regeneration.

256. The method of claim 255, wherein the biologically active agent is selected from the group consisting of an angiogenesis inhibitor, paclitaxel, enzymes, receptor antagonists, receptor agonists, hormones, growth factors, small molecules, autogenous bone marrow, antibiotics, antimicrobial agents, antibodies, cytokines, bone morphogenic proteins, and growth factors.

257. A method of repairing damaged soft tissues in a patient comprising placing into contact with the damaged soft tissues, a composition comprising components (i), (ii), and (iii) or a partial reaction product of components (i), (ii), and (iii), wherein components (i), (ii), and (iii) are comprised of:
(i) a first hydrophilic polymer;
(ii) a crosslinkable component A having m nucleophilic groups, wherein m≧2; and
(iii) a crosslinkable component B having n electrophilic groups capable of reaction with the m nucleophilic groups to form covalent bonds, wherein n>2 and m+n>4,
wherein each of components A and B is biocompatible and nonimmunogenic, at least one of the components A and B is a second hydrophilic polymer, and reaction of the components results in a biocompatible, nonimmunogenic, crosslinked matrix.

258. The method of claim 257, wherein the damaged soft tissue is connective tissue.

259. The method of claim 257, wherein the connective tissue is selected from the group consisting of articular ligaments, tendons, muscles, fat, or a soft tissue implant.

260. The method of claim 259, wherein the soft tissue implants are cosmetic implants.

261. The method of claim 260, wherein the cosmetic implants are breast implants or facial implants.

262. The method of claim 257, wherein the composition further comprises a biologically active agent.

263. A kit for repairing damaged cartilage tissue, wherein the kit comprises:
(a) a first hydrophilic polymer;
(b) a crosslinkable component A having m nucleophilic groups, wherein m≧2; and
(c) a crosslinkable component B having n electrophilic groups capable of reaction with the m nucleophilic groups to form covalent bonds, wherein n≧2 and m+n>4,
wherein each of components A and B is biocompatible and nonimmunogenic, at least one of components A and B is a second hydrophilic polymer, and reaction of the components results in a biocompatible, nonimmunogenic, crosslinked matrix.

264. The kit of claim 263, wherein each of components A and B comprises polyalkeleneoxide.

265. The kit of claim 263, wherein the polyalkyleneoxide is a poly(ethylene oxide).

266. The kit of claim 263, wherein components A and B are the same.

267. The kit of claim 263, wherein components A and B are in admixture.

268. The kit of claim 263, wherein the admixture is in a solid form.

269. The kit of claim 263, further comprising a device for mixing (a), (b), and (c) and delivering (a), (b), and (c) or a partial reaction product thereof to the damaged cartilage tissue.

270. The kit of claim 269, wherein the device is configured to spray material onto a surface of the damaged cartilage tissue.

271. The kit of claim 269, wherein the device is configured to deliver material onto a surface of the damaged cartilage tissue as a liquid, gel, or suspension.

272. The kit of claim 263, wherein the first hydrophilic polymer is methylated collagen dissolved or suspended in aqueous solution of pH less than 7.

273. The kit of claim 263, further comprising (d), wherein (d) comprises an aqueous solution of pH greater than 7.

274. The kit of claim 272, wherein component A is (pentaerythritol tetrakis[mercaptoethyl poly(oxyethylene) ether] and component B is pentaerythritol tetrakis [1-(1′-oxo-5-succimidylpentanoate)-2-poly(oxyethylene) ether.
